(19) United States
(12) Patent Application Publication
(10) Pub. No.: US 2005/0239733 A1
(43) Pub. Date: Oct. 27, 2005

(54) Sequence requirements for inhibitory oligonucleotides

(75) Inventors: Marion Jurk, Duesseldorf (DE); Jorg Vollmer, Duesseldorf (DE); Arthur M. Krieg, Wellesley, MA (US); Eugen Uhlmann, Glashuetten (DE)

Correspondence Address: WOLF GREENFIELD & SACKS, PC;FEDERAL RESERVE PLAZA, 600 ATLANTIC AVENUE, BOSTON, MA, 02210-2211, US

(73) Assignees: Coley Pharmaceutical GmbH, Langenfeld (DE); Coley Pharmaceutical Group, Inc., Wellesley, MA (US)

(21) Appl. No.: 10/977,560

(22) Filed: Oct. 29, 2004

Related U.S. Application Data
(60) Provisional application No. 60/516,221, filed on Oct. 31, 2003.

(51) Int. Cl. A61K 48/00; C07H 21/04

(52) U.S. Cl. 514/44; 536/23.2

(57) ABSTRACT

Novel oligonucleotides having immune inhibitory effects, and methods for their use, are provided. The inhibitory oligonucleotides include those that specifically inhibit certain Toll-like receptors, including TLR7, TLR8, and TLR9. Certain of the immunoinhibitory oligonucleotides inhibit a combination of TLRs selected from TLR7, TLR8, and TLR9. Inhibitors of TLR9 are characterized by a 5′ CC dinucleotide appropriately spaced upstream of a G-rich oligomer. Inhibitors of TLR8 include specific simple dinucleotides and oligonucleotides ending at their 3′ termini with the specific dinucleotides. TLR7 inhibitors include oligonucleotides having a phosphorothioate backbone. Also provided are combinations and conjugates involving the inhibitory oligonucleotides of the invention and other agents, where the other agents include TLR agonists and antigens. Compositions of the invention can be used to shape an immune response, reduce unwanted specific TLR-mediated immunostimulation, and to treat conditions including allergy, asthma, infection, and cancer.

### RELATED APPLICATION

This application claims priority under 35 U.S.C. 119 to U.S. Provisional Application Ser. No. 60/516,221, filed Oct. 31, 2003, the entire content of which is incorporated herein by reference.

### BACKGROUND OF THE INVENTION

Reaction to certain motifs in bacterial DNA is an important function of natural immunity. Bacterial DNA has long been known to be mitogenic for mammalian B lymphocytes (B cells), whereas mammalian DNA generally is not. The discovery that this immune recognition was directed to specific DNA sequences centered on a motif containing an unmethylated CpG dinucleotide opened the field to molecular immunologic approaches. Krieg AM et al. (1995) Nature 374:546-9. The immunostimulatory effects of so-called CpG DNA can be reproduced using synthetic oligodeoxynucleotides (ODN) containing CpG dinucleotides in the context of certain preferred flanking sequence, a CpG motif. CpG-containing ODN (CpG-ODN) have been reported to exert a number of effects on various types of cells of the immune system, including protecting primary B cells from apoptosis, promotion of cell cycle entry, and skewing an immune response toward a Th1-type immune response, e.g., induction of interleukin 6 (IL-6), interleukin 12 (IL-12), gamma interferon (IFN-γ), activation of antigen-specific cytolytic T lymphocytes (CTL), and induction in the mouse of IgG2a.

Recently it has been reported that the immunomodulatory effects of CpG DNA involve signaling by Toll-like receptor 9 (TLR9). It is believed that CpG DNA is internalized into a cell via a sequence-nonspecific pathway and traffics to the endosomal compartment, where it interacts with TLR9 in a sequence-specific manner. TLR9 signaling pathways lead to induction of a number of immune-function related genes, including notably NF-κB, among others.

The TLRs are a large family of receptors that recognize specific molecular structures that are present in pathogens (pathogen-associated molecular patterns or PAMPs) and are also termed pattern recognition receptors (PRRs). Immune cells expressing PRRs are activated upon recognition of PAMPs and trigger the generation of optimal adaptive immune responses. PRRs consisting of 10 different TLR subtypes, TLR1 to TLR10, have been described. Such TLRs have been described to be involved in the recognition of double-stranded RNA (TLR3), lipopolysaccharide (LPS) (TLR4), bacterial flagellin (TLR5), small anti-viral compounds (TLR7 and TLR8), and bacterial DNA or CpG ODN (TLR9). Reviewed in Uhlmann et al. (2003) Curr Opin Drug Discov Devel 6:204-17. In addition, RNA molecules were recently identified that are believed to interact with and signal through TLR7 and TLR8. International patent application PCT/US03/10406. Such immunostimulatory RNA molecules are believed to have a base sequence that includes at least one guanine and at least one uracil. The immunostimulatory G,U-rich RNA does not require a CpG motif as described for TLR9. The corresponding class of RNA molecules found in nature is believed to be present in ribosomal RNA (rRNA), transfer RNA (tRNA), messenger RNA (mRNA), and viral RNA (vRNA).

Following the discovery of immunostimulatory CpG DNA, a number of reports appeared describing short DNA sequences with immunoinhibitory effects. It has long been known that poly-G sequences were immunoinhibitory. Published PCT patent application WO 00/14217 describes ODN containing an inhibitory motif N₁N₂GN₃G in which at least any two of N₁, N₂, and N₃ are G (guanosine). Krieg and colleagues described a group of inhibitory 15-mer ODN, having three or four consecutive G, that blocked apoptosis protection and cell-cycle entry induced by stimulatory ODN. Lenert P et al. (2001) Antisense Nucleic Acid Drug Dev 11:247-56; Stunz LL et al. (2002) Eur J Immunol 32:1212-22; Lenert P et al. (2003) Antisense Nucleic Acid Drug Dev 13:143-50. The immunoinhibitory effect of these ODN was reported to be specific for CpG-ODN and to involve a mechanism other than simple competition for cellular uptake. Stunz LL et al. (2002) Eur J Immunol 32:1212-22. Independently, Klinman and colleagues reported a single immunoinhibitory ODN. Zeuner RA et al. (2002) Arthritis Rheum 46:2219-24; Yamada H et al. (2002) J Immunol 169:5590-4.

### SUMMARY OF THE INVENTION

It has now been discovered by the applicants that certain nucleic acid molecules selectively inhibit signaling mediated by Toll-like receptors TLR9, TLR8, and TLR7. Certain of these nucleic acid molecules are oligodeoxynucleotides (inhibitory ODN) ranging in length from 2 to about 50 nucleotides. While certain of the inhibitory ODN are selectively inhibitory with respect to just one of TLR9, TLR8, or TLR7, certain of the inhibitory ODN are selectively inhibitory with respect to two or more of TLR9, TLR8, and TLR7. The inhibitory ODN can be used alone, in combination with one another, or in combination with another agent, e.g., an immunostimulatory CpG nucleic acid molecule or TLR agonist, to shape an immune response in vivo or in vitro.

As is described in greater detail below, the applicants have discovered that certain oligonucleotides characterized by a 5′ cytosine-cytosine (CC) dinucleotide and a downstream 3′ G-rich sequence are inhibitory toward signaling by TLR9. In certain embodiments the C's, G's, or both C's and G's can be cytosine or guanosine derivatives. Some but not all such inhibitory ODN also inhibit signaling by TLR7, TLR8, or both TLR7 and TLR8.

Also as is described in greater detail below, the present applicants have discovered that certain oligonucleotides characterized by a GK dinucleotide, by itself or positioned at the 3′ terminus of an oligonucleotide, are inhibitory toward signaling by TLR8. In one embodiment the GK dinucleotide is GT. In one embodiment the G of the GK dinucleotide can be a guanosine derivative. Some but not all such inhibitory ODN also inhibit signaling by TLR7, TLR9, or both TLR7 and TLR9.

It has also been discovered by the present applicants that any phosphorothioate ODN is inhibitory toward signaling by TLR7. Some but not all such inhibitory ODN also inhibit signaling by TLR8, TLR9, or both TLR8 and TLR9.

With respect to inhibition of TLR9 signaling, it has now been discovered by the instant applicants that the inhibitory effects of immunoinhibitory ODN (inhibitory ODN) are potently and unexpectedly enhanced by the presence of a cytosine-cytosine (CC) dinucleotide upstream of (i.e., 5′ to) and properly spaced from a G-rich sequence. Surprisingly, substitution of the 5′ CC dinucleotide by any other dinucleotide significantly reduces the inhibitory effects of a given inhibitory ODN. However, the instant applicants have also discovered that either or both of the cytosines of the 5′ CC dinucleotide can optionally be replaced with a cytosine derivative, including, among others, 5-methylcytosine, without significant loss of inhibitory effect. Furthermore, any or all of the guanosines of the G-rich sequence can optionally be replaced with a guanosine derivative, notably a deazaguanosine, also without significant loss of inhibitory effect. Combinations of C derivatives and of G derivatives can also be used in individual inhibitory ODN, without significant loss of inhibitory effect, provided the overall sequence motif is preserved.

It has also now been discovered by the instant applicants that nanomolar to micromolar concentrations of the inhibitory ODN of the invention effectively inhibit CpG-DNA-induced TLR9 signaling.

Certain of the inhibitory ODN of the invention thus are useful whenever it is desirable to inhibit CpG-DNA-induced immunostimulation. Furthermore, the inhibitory ODN of the invention thus are useful whenever it is desirable to inhibit CpG-DNA-induced TLR9 signaling. The inhibitory ODN of the invention are useful in vitro and in vivo in methods for reducing CpG-DNA-induced immunostimulation and for treating conditions involving CpG-DNA-induced immunostimulation. In addition, the inhibitory ODN of the invention can be used in a method for preparation of a medicament for treating a condition involving CpG-DNA-induced DNA-induced immunostimulation in a subject.

In addition, certain of the inhibitory ODN of the invention are useful whenever it is desirable to inhibit RNA- or small anti-viral compound (e.g., R-848)-induced immunostimulation. Furthermore, the inhibitory ODN of the invention thus are useful whenever it is desirable to inhibit RNA- or small anti-viral compound-induced TLR7 and/or TLR8 signaling. The inhibitory ODN of the invention are useful in vitro and in vivo in methods for reducing RNA-induced immunostimulation and for treating conditions involving RNA-induced immunostimulation. The inhibitory ODN of the invention can be used in a method for preparation of a medicament for treating a condition involving RNA-induced immunostimulation in a subject.

The present invention can be used for preventing and treating septic shock, inflammation, allergy, asthma, graft rejection, graft-versus host disease (GvHD), autoimmune diseases, Th1- or Th2-mediated diseases, bacterial infections, parasitic infections, spontaneous abortions, and tumors. The present invention can be used generally to inhibit activation of all cells expressing the relevant TLRs, and more specifically to inhibit activation of antigen-presenting cells, B cells, plasmacytoid dendritic cells (pDCs), monocytes, monocyte-derived cells, eosinophils, and neutrophils.

ODN of the present invention can also be used as antidotes related to diseases caused by specific therapeutic compounds like TLR9 agonists (ODN or small molecules) or TLR8 agonists (like resiquimod). An advantage of the present invention is that in some embodiments the use of a certain inhibitory ODN to inhibit one TLR does not inhibit activation of another TLR. Thus for example treatment with an antidote for TLR8 activation with an inhibitory ODN specific for TLR8 (e.g., ODN 443, 444, or 445) does not result in suppression of TLR9. Activation of the immune system by a TLR9-related pathogen or other TLR9 agonist is still possible which will be beneficial to the treated individual.

In one aspect the invention provides a composition including an isolated immunoinhibitory nucleic acid molecule including a sequence
XₐCCN₁N₂N₃Y_{b}N₄GGGZ_{c} (SEQ ID NO:1)
   wherein each C is cytidine or a derivative thereof, wherein at least one C is a cytidine derivative; each G is guanosine or a deaza derivative thereof; Xₐ is any nucleotide sequence a nucleotides long, wherein a is an integer between 0-12, inclusive, and each nucleotide is selected independently of any other in Xₐ; Y_{b} is any nucleotide sequence b nucleotides long, wherein b is an integer between 0-21, inclusive, and each nucleotide is selected independently of any other in Y_{b}; Z_{c} is any nucleotide sequence c nucleotides long, wherein c is an integer between 0-12, inclusive, and each nucleotide is selected independently of any other in Z_{c}; and N₁, N₂, N₃, and N₄ are each independently any nucleotide.

In various embodiments according to this aspect of the invention, N₁, is T (thymidine); N₂ is G; N₁N₂ is TG; N₄ is G; Xₐ is T, or any combination thereof.

In one embodiment according to this aspect of the invention, each C is a cytidine derivative.

In one embodiment according to this aspect of the invention, at least one C is 5-methylcytidine.

In one embodiment according to this aspect of the invention, at least one G is 7-deazaguanosine.

In one embodiment according to this aspect of the invention, each G is 7-deazaguanosine.

In one embodiment according to this aspect of the invention, b is a smallest integer between 0-21, inclusive, able to conform to the sequence.

In one embodiment according to this aspect of the invention, the immunoinhibitory nucleic acid molecule has a phosphorothioate backbone.

In one embodiment according to this aspect of the invention, the sequence includes XₐCCTGN₃Y_{b}GGGGZ_{c} (SEQ ID NO:3). In various embodiments according to this aspect of the invention, the sequence includes TCCTGGCGGGGAAGT (SEQ ID NO:4), GCCTGGCGGGGAAGT (SEQ ID NO:5), ACCTGGCGGGGAAGT (SEQ ID NO:6), CCCTGGCGGGGAAGT (SEQ ID NO:7), TCCCGGCGGGGAAGT (SEQ ID NO:8), TCCGGGCGGGGAAGT (SEQ ID NO:9), TCCTAGCGGGGAAGT (SEQ ID NO:10), TCCTGGAGGGGAAGT (SEQ ID NO:11), TCCTAGCGGGGGCGTCCTAT (SEQ ID NO:12), or CCTCAAGCTTGAGGGG (SEQ ID NO:13).

In various embodiments according to this aspect of the invention, the sequence is TCCTGGCGGGGAAGT (SEQ ID NO:4), GCCTGGCGGGGAAGT (SEQ ID NO:5), ACCTGGCGGGGAAGT (SEQ ID NO:6), CCCTGGCGGGGAAGT (SEQ ID NO:7), TCCCGGCGGGGAAGT (SEQ ID NO:8), TCCGGGCGGGGAAGT (SEQ ID NO:9), TCCTAGCGGGGAAGT (SEQ ID NO:10), TCCTGGAGGGGAAGT (SEQ ID NO:11), TCCTAGCGGGGGCGTCCTAT (SEQ ID NO:12), or CCTCAAGCTTGAGGGG (SEQ ID NO:13).

In one embodiment the composition according to this aspect of the invention further includes a pharmaceutically acceptable carrier.

The invention in one aspect provides a composition including an isolated immunoinhibitory nucleic acid molecule including a sequence
XₐCCN₁N₂N₃Y_{b}N₄GGGZ_{c} (SEQ ID NO:1)
   wherein each C is cytidine or a derivative thereof; each G is guanosine or a deaza derivative thereof; Xₐ is any nucleotide sequence a nucleotides long, wherein a is an integer between 0-12, inclusive, and each nucleotide is selected independently of any other in Xₐ; Y_{b} is any nucleotide sequence b nucleotides long, wherein b is an integer between 8-21, inclusive, and each nucleotide is selected independently of any other in Y_{b}; Z_{c} is any nucleotide sequence c nucleotides long, wherein c is an integer between 0-12, inclusive, and each nucleotide is selected independently of any other in Z_{c}; and N₁, N₂, N₃, and N₄ are each independently any nucleotide.

In various embodiments according to this aspect of the invention, N₁ is T (thymidine); N₂ is G; N₁N₂ is TG; N₄ is G; Xₐ is T, or any combination thereof.

In one embodiment according to this aspect of the invention, each C is a cytidine derivative.

In one embodiment according to this aspect of the invention, at least one C is 5-methylcytidine.

In one embodiment according to this aspect of the invention, at least one G is 7-deazaguanosine.

In one embodiment according to this aspect of the invention, each G is 7-deazaguanosine.

In one embodiment according to this aspect of the invention, the immunoinhibitory nucleic acid molecule has a phosphorothioate backbone.

In one embodiment the sequence includes XₐCCTGN₃Y_{b}GGGGZ_{c} (SEQ ID NO:3).

In one embodiment the sequence includes XₐCCTN₂GY_{b}GGGGZ_{c} (SEQ ID NO:58), wherein N₂ is not G.

In various embodiments according to this aspect of the invention, the sequence includes TCCTGTGTGTGTGTCGGGGAAGT (SEQ ID NO:14), TCCTGTGTGTGTGTGTCGGGGAAGT (SEQ ID NO:15), or TCCTGTGTGTGTGTGTGTCGGGGAAGT (SEQ ID NO:16).

In various embodiments according to this aspect of the invention, the sequence is TCCTGTGTGTGTGTCGGGGAAGT (SEQ ID NO:14), TCCTGTGTGTGTGTGTCGGGGAAGT (SEQ ID NO:15), or TCCTGTGTGTGTGTGTGTCGGGGAAGT (SEQ ID NO:16).

In one embodiment the composition according to this aspect of the invention further includes a pharmaceutically acceptable carrier.

The invention in one aspect provides a composition including an isolated immunoinhibitory nucleic acid molecule including a sequence
XₐCCN₁Y_{b}N₂N₃N₄N₅GGZ_{c} (SEQ ID NO:2)
   wherein:

N₂N₃N₄N₅GG is selected from GGN₄N₅GG, GN₃N₄GGG, N₂GN₄GGG, and N₂N₃N₄GGG; each C is cytidine or a derivative thereof; each G is guanosine or a deaza derivative thereof; Xₐ is any nucleotide sequence a nucleotides long, wherein a is an integer between 0-12, inclusive, and each nucleotide is selected independently of any other in Xₐ; Y_{b} is any nucleotide sequence b nucleotides long, wherein b is an integer between 8-21, inclusive, and each nucleotide is selected independently of any other in Y_{b}; Z_{c} is any nucleotide sequence c nucleotides long, wherein c is an integer between 0-12, inclusive, and each nucleotide is selected independently of any other in Z_{c}; and N₁, N₂, N₃, N₄, and N₅ are each independently any nucleotide.

In one embodiment according to this aspect of the invention at least one C is 5-methylcytidine.

In one embodiment according to this aspect of the invention at least one G is 7-deazaguanosine.

In one embodiment according to this aspect of the invention each G is 7-deazaguanosine.

In one embodiment according to this aspect of the invention, N₁ is T.

In one embodiment according to this aspect of the invention, Xₐ is T.

In one embodiment according to this aspect of the invention, the immunoinhibitory nucleic acid molecule has a phosphorothioate backbone.

In various embodiments according to this aspect of the invention, the sequence includes TCCTGTGTGTGTGTCGGGGAAGT (SEQ ID NO:14), TCCTGTGTGTGTGTGTCGGGGAAGT (SEQ ID NO: 15), orTCCTGTGTGTGTGTGTGTCGGGGAAGT (SEQ ID NO:16).

In various embodiments according to this aspect of the invention, the sequence is TCCTGTGTGTGTGTCGGGGAAGT (SEQ ID NO: 14), TCCTGTGTGTGTGTGTCGGGGAAGT (SEQ ID NO:15), orTCCTGTGTGTGTGTGTGTCGGGGAAGT (SEQ ID NO:16).

In one embodiment the composition according to this aspect of the invention further includes a pharmaceutically acceptable carrier.

The invention further provides a composition including a conjugate of an antigen and an isolated immunoinhibitory nucleic acid molecule of the invention.

The invention further provides a composition including a conjugate of a TLR agonist and an isolated immunoinhibitory nucleic acid molecule of the invention.

The invention further provides a composition of the invention wherein Z_{c} is not K when c is 1 and wherein Z_{c} does not terminate with GK when c is an integer between 2-12, inclusive, wherein G is chosen from guanosine and 7-deazaguanosine and K is chosen from thymidine (T), uracil (U), and G.

The invention further provides a composition of the invention wherein Z_{c} is not T when c is 1 and wherein Z_{c} does not terminate with GT when c is an integer between 2-12, inclusive, wherein G is guanosine and T is thymidine.

The invention further provides a composition of the invention wherein Z_{c} is K when c is 1 and wherein Z_{c} terminates with GK when c is an integer between 2-12. inclusive, wherein G is chosen from guanosine and 7-deazaguanosine and K is chosen from thymidine (T), uracil (U), and G.

The invention further provides a composition of the invention wherein Z_{c}is T when c is 1 and wherein Z_{c}terminates with GT when c is an integer between 2-12, inclusive, wherein G is guanosine and T is thymidine.

The invention further provides a composition of the invention wherein Z_{c}terminates with 7T when c is an integer between 2-12, inclusive, wherein 7 is 7-deazaguanosine.

The invention in a further aspect provides a method for inhibiting TLR signaling. The method according to this aspect of the invention involves the step of contacting a cell or a population of cells expressing at least one TLR chosen from TLR7, TLR8, TLR9, or any combination thereof, with an effective amount of a composition of the invention to inhibit signaling by TLR7, TLR8, and TLR9.

The invention further provides a method for inhibiting TLR signaling in a subject. The method according to this aspect of the invention includes the step of administering to a subject an effective amount of a composition of the invention to inhibit signaling by TLR7, TLR8, and TLR9 in the subject.

The invention in a further aspect provides a method for inhibiting TLR9 signaling. The method according to this aspect of the invention includes the step of contacting a cell or a population of cells expressing TLR9 with an effective amount of a composition of the invention, to inhibit signaling by TLR9.

The invention further provides a method for inhibiting TLR9 signaling in a subject. The method according to this aspect of the invention includes the step of administering to a subject an effective amount of a composition of the invention, to inhibit signaling by TLR9 in the subject.

The invention in a further aspect provides a method for inhibiting TLR8 signaling. The method according to this aspect of the invention includes the step of contacting a cell or a population of cells expressing TLR8 with an effective amount of a GK dinucleotide, wherein G is chosen from guanosine and 7-deazaguanosine and K is chosen from thymidine, uracil, and guanosine, to inhibit signaling by TLR8.

The invention further provides a method for inhibiting TLR8 signaling in a subject. The method according to this aspect of the invention includes the step of administering to a subject an effective amount of a GK dinucleotide, wherein G is chosen from guanosine and 7-deazaguanosine and K is chosen from thymidine, uracil, and guanosine, to inhibit signaling by TLR8.

The invention in a further aspect provides a method for inhibiting TLR8 signaling. The method according to this aspect of the invention includes the step of contacting a cell or a population of cells expressing TLR8 with an effective amount of a GT dinucleotide, wherein G is guanosine T is thymidine, to inhibit signaling by TLR8.

The invention further provides a method for inhibiting TLR8 signaling in a subject. The method according to this aspect of the invention includes the step of administering to a subject an effective amount of a GT dinucleotide, wherein G is guanosine T is thymidine, to inhibit signaling by TLR8 in the subject.

The invention in a further aspect provides a method for inhibiting TLR signaling. The method according to this aspect of the invention includes the steps of contacting a cell or a population of cells expressing at least one TLR chosen from TLR7, TLR8, TLR9, or any combination thereof, with an effective amount of a composition of the invention to inhibit signaling by TLR9; and contacting the cell or population of cells with an effective amount of a phosphorothioate oligonucleotide 2-40 nucleotides long including a 3′ end terminating with GK, wherein G is chosen from guanosine and 7-deazaguanosine and K is chosen from thymidine, uracil, and guanosine, to inhibit signaling by TLR7 and TLR8.

The invention further provides a method for inhibiting TLR signaling in a subject. The method according to this aspect of the invention includes the steps of administering to a subject an effective amount of a composition of the invention to inhibit signaling by TLR9; and administering to a subject an effective amount of a phosphorothioate oligonucleotide 2-40 nucleotides long including a 3′ end terminating with GK, wherein G is chosen from guanosine and 7-deazaguanosine and K is chosen from thymidine, uracil, and guanosine, to inhibit signaling by TLR7 and TLR8 in the subject.

The invention in a further aspect provides a method for inhibiting TLR9 signaling without inhibiting TLR8 signaling. The method according to this aspect of the invention includes the step of contacting a cell or a population of cells expressing TLR8 and TLR9 with an effective amount of a composition of the invention to inhibit signaling by TLR9 without inhibiting signaling by TLR8.

The invention further provides a method for inhibiting TLR9 signaling without inhibiting TLR8 signaling in a subject. The method according to this aspect of the invention includes the step of administering to a subject an effective amount of a composition of the invention to inhibit signaling by TLR9 without inhibiting signaling by TLR8 in the subject.

The invention in a further aspect provides a method for promoting TLR9 signaling and inhibiting TLR8 signaling. The method according to this aspect of the invention includes the steps of contacting a cell or population of cells expressing TLR8 and TLR9 with an effective amount of an immunostimulatory CpG nucleic acid molecule, wherein the immunostimulatory CpG nucleic acid molecule does not have a 3′ end terminating with GT, to promote TLR9 signaling; and contacting the cell or population of cells with an effective amount of an oligonucleotide 2-40 nucleotides long, wherein the oligonucleotide includes a 3′ end terminating with GT, to inhibit signaling by TLR8.

The invention further provides a method for promoting TLR9 signaling and inhibiting TLR8 signaling in a subject. The method according to this aspect of the invention includes the steps of administering to a subject an effective amount of an immunostimulatory CpG nucleic acid molecule, wherein the immunostimulatory CpG nucleic acid molecule does not have a 3′ end terminating with GT, to promote TLR9 signaling in the subject; and administering to the subject an effective amount of an oligonucleotide 2-40 nucleotides long, wherein the oligonucleotide includes a 3′ end terminating with GT, to inhibit signaling by TLR8 in the subject.

The invention in a further aspect provides a method for promoting TLR8 signaling and inhibiting TLR9 signaling. The method according to this aspect of the invention includes the steps of contacting a cell or population of cells expressing TLR8 and TLR9 with an effective amount of a TLR8 signaling agonist to promote TLR8 signaling; and contacting the cell or population of cells with an effective amount of a composition of the invention, to inhibit TLR9 signaling.

The invention further provides a method for promoting TLR8 signaling and inhibiting TLR9 signaling in a subject. The method according to this aspect of the invention includes the steps of administering to a subject an effective amount of a TLR8 signaling agonist to promote TLR8 signaling in the subject; and administering to the subject an effective amount of a composition of the invention, to inhibit TLR9 signaling in the subject.

The invention in a further aspect provides a method for reducing an immunostimulatory effect of a CpG nucleic acid molecule. The method according to this aspect of the invention includes the step of contacting an immune cell that is sensitive to a CpG nucleic acid molecule with an effective amount of an isolated immunoinhibitory nucleic acid molecule of the invention to reduce an immunostimulatory effect of the CpG nucleic acid molecule on the immune cell to a level below that which would occur without the contacting.

In one embodiment according to this aspect of the invention, the immunostimulatory effect is Th1-like skewing.

In one embodiment according to this aspect of the invention, the contacting occurs at least 24 hours before the immune cell contacts the CpG nucleic acid molecule.

In one embodiment according to this aspect of the invention, the contacting occurs within 24 hours of the immune cell contacting the CpG nucleic acid molecule.

In one embodiment according to this aspect of the invention, the contacting occurs at least 24 hours after the immune cell contacts the CpG nucleic acid molecule.

The invention further provides a method for treating a condition associated with CpG-mediated immunostimulation in a subject. The method according to this aspect of the invention includes the step of administering to a subject having or at risk of developing a condition associated with CpG-mediated immunostimulation an effective amount of an isolated immunoinhibitory nucleic acid molecule of the invention to treat the condition.

In one embodiment according to this aspect of the invention, the condition is a Th1-like like immune response.

In one embodiment according to this aspect of the invention, the condition is an autoimmune disease.

In one embodiment according to this aspect of the invention, the condition is inflammation.

In one embodiment according to this aspect of the invention, the condition is infection with a CpG-containing microbe.

In one embodiment according to this aspect of the invention, the condition is sepsis.

In one embodiment according to this aspect of the invention, the administering occurs at least 24 hours before the subject contacts a source of CpG nucleic acid molecule.

In one embodiment according to this aspect of the invention, the administering occurs within 24 hours of the subject contacting a source of CpG nucleic acid molecule.

In one embodiment according to this aspect of the invention, the administering occurs at least 24 hours after the subject contacts a source of CpG nucleic acid molecule.

### BRIEF DESCRIPTION OF THE DRAWINGS

FIG. 1 is a graph depicting inhibition of CPG ODN 2006-mediated NF-κB activation in hTLR9-LUC-293 cells by various ODN. ODN include CpG ODN 2006 (SEQ ID NO:17), random 15-mer ODN 605, and inhibitory ODN 2088 (SEQ ID NO:4), ODN 673 (SEQ ID NO:18), and ODN 674 (SEQ ID NO:19).

FIG. 2 is a graph depicting inhibition of CpG ODN 2006-mediated NF-κB activation in hTLR9-LUC-293 cells by various ODN of different length. ODN include inhibitory ODN 2088 (SEQ ID NO:4), ODN 494 (SEQ ID NO:14), ODN 495 (SEQ ID NO:15), and ODN 497 (SEQ ID NO:16). Values for b refer to the number of nucleotides Y_{b} in the formula XₐCCN₁N₂N₃Y_{b}N₄GGGZ_{c}(SEQ ID NO:1).

FIG. 3 is a graph depicting inhibition of R-848-mediated NF-κB activation in hTLR8-LUC-293 cells in the presence of varied concentrations of ODN 2088 (SEQ ID NO:4).

FIG. 4 is a trio of graphs depicting different inhibition patterns of specific ODN for hTLR8 and hTLR9. FIG. 4A depicts remaining TLR activity after 16 hours culture in the presence of agonist 0.156 μM CpG ODN 2006 (SEQ ID NO:17; for TLR9) or 50 μM R-848 (for TLR8) and antagonist ODN 2088 (SEQ ID NO:4) over a range of agonist:antagonist concentration ratios. FIG. 4B depicts remaining TLR activity after 16 hours culture in the presence of agonist 0.156 μM CpG ODN 2006 (for TLR9) or 50 μM R-848 (for TLR8) and antagonist ODN 962 (SEQ ID NO:20) over a range of agonist:antagonist concentration ratios. FIG. 4C depicts remaining TLR activity after 16 hours culture in the presence of agonist 0.156 μM CpG ODN 2006 (for TLR9) or 50 μM R-848 (for TLR8) and antagonist ODN 969 (SEQ ID NO:21) over a range of agonist:antagonist concentration ratios.

FIG. 5 is a graph depicting ODN sequence dependence of inhibition of hTLR8. Indicated ODN include ODN 2088 (SEQ ID NO:4), D₁₃GT, GTN₁₃, N₆GTN₇, N₁₅, N₁₃GT, and GT.

FIG. 6 is a graph depicting terminal 3′ dinucleotide sequence dependence of inhibition of hTLR8. Indicated ODN include ODN 2088 (GT; SEQ ID NO:4), and the following variants of ODN 2088: ODN 458 (GA; SEQ ID NO:22), ODN 459 (GC; SEQ ID NO:23), ODN 460 (GG; SEQ ID NO:24), ODN 461 (AT; SEQ ID NO:25), ODN 462 (CT; SEQ ID NO:26), 463 (TT; SEQ ID NO:27), 604 (GU; SEQ ID NO:28), and ODN 599 (7T; SEQ ID NO:29).

FIG. 7 is a graph depicting percent remaining R-848 activity in the presence of various indicated dinucleotides or ODN 2088 (SEQ ID NO:4).

FIG. 8 is a group of four graphs depicting inhibition of TLR7, TLR8, and TLR9 by phosphorothioate ODN having different sequence motifs. FIG. 8A depicts the inhibitory effect of ODN 2088 (SEQ ID NO:4). FIG. 8B depicts the inhibitory effect of random sequence phosphorothioate 16-mer. FIG. 8C depicts the inhibitory effect of the TLR9 inhibitory ODN motif NCCNNNNNGGGNNNN (SEQ ID NO:19). FIG. 8D depicts the inhibitory effect of the TLR8 inhibitory ODN NN NNNNNNNNNNNNNGT.

FIG. 9 is a a graph depicting inhibition of interferon alpha (IFN-α) secretion by peripheral blood mononuclear cells (PBMC) incubated in the presence of 0.5 μM CpG ODN 2395 (SEQ ID NO:30) alone and in combination with various indicated concentrations of ODN 2088 (SEQ ID NO:4), ODN 673 (SEQ ID NO:18), ODN 674 (SEQ ID NO:19), ODN 467 (NNNNNNNNNNNNNNGT), or ODN 223 (SEQ ID NO:31).

TABLE OF OLIGONUCLEOTIDES

| | | |
|---|---|---|
| ODN | Sequence | SEQ ID NO: |

| | | |
|---|---|---|
| | XₐCCN₁N₂N₃Y_{b}N₄GGGZ_{c} | 1 |
| | XₐCCN₁Y_{b}N₂N₃N₄N₅GGZ_{c} | 2 |
| | XₐCCTGN₃Y_{b}GGGGZ_{c} | 3 |
| 2088 | TCCTGGCGGGGAAGT | 4 |
| 940 | GCCTGGCGGGGAAGT | 5 |
| 941 | ACCTGGCGGGGAAGT | 6 |
| 942 | CCCTGGCGGGGAAGT | 7 |
| 155 | TCCCGGCGGGGAAGT | 8 |
| 156 | TCCGGGCGGGGAAGT | 9 |
| | TCCTAGCGGGGAAGT | 10 |
| 2114 | TCCTGGAGGGGAAGT | 11 |
| | TCCTAGCGGGGGCGTCCTAT | 12 |
| | CCTCAAGCTTGAGGGG | 13 |
| 494 | TCCTGTGTGTGTGTCGGGGAAGT | 14 |
| 495 | TCCTGTGTGTGTGTGTCGGGGAAGT | 15 |
| 497 | TCCTGTGTGTGTGTGTGTCGGGGAAGT | 16 |
| 2006 | TCGTCGTTTTGTCGTTTTGTCGTT | 17 |
| 673 | NCCNNNNGGGGNNNN | 18 |
| 674 | NCCNNNNNGGGNNNN | 19 |
| 962 | CTGGCGGGGAAGT | 20 |
| 969 | TCCTGGCGGGGAA | 21 |
| 458 | TCCTGGCGGGGAAGA | 22 |
| 459 | TCCTGGCGGGGAAGC | 23 |
| 460 | TCCTGGCGGGGAAGG | 24 |
| 461 | TCCTGGCGGGGAAAT | 25 |
| 462 | TCCTGGCGGGGAACT | 26 |
| 463 | TCCTGGCGGGGAATT | 27 |
| 604 | TCCTGGCGGGGAAGU | 28 |
| 599 | TCCTGGCGGGGAA7T | 29 |
| 2395 | TCGTCTTTTCGGCGCGCGCCG | 30 |
| 223 | CCTTGTTGGG | 31 |
| 159 | TZZTGGCGGGGAAGT | 32 |
| 166 | TCCTGGCGGGGAAGT | 33 |
| 443 | TGCTGGCGGGGAAGT | 34 |
| 444 | TGATGGCGGGGAAGT | 35 |
| 445 | TGTTGGCGGGGAAGT | 36 |
| 493 | TCCTGLGGGGAAGT | 37 |
| 492 | TCCTGLLGGGGAAGT | 38 |
| 959 | TAATGGCGGGGAAGT | 39 |
| 074 | TACTGGCGGGGAAGT | 40 |
| 441 | TAGTGGCGGGGAAGT | 41 |
| 442 | TATTGGCGGGGAAGT | 42 |
| 073 | TCATGGCGGGGAAGT | 43 |
| 435 | TCGTGGCGGGGAAGT | 44 |
| 437 | TCTTGGCGGGGAAGT | 45 |
| 436 | TGGTGGCGGGGAAGT | 46 |
| 439 | TTATGGCGGGGAAGT | 47 |
| 440 | TTCTGGCGGGGAAGT | 48 |
| 438 | TTGTGGCGGGGAAGT | 49 |
| 072 | TTTTGGCGGGGAAGT | 50 |
| 157 | TCCAGGCGGGGAAGT | 51 |
| 961 | CCTGGCGGGGAAGT | 52 |
| 484 | TbCbCTGGCGGGGAAGT | 53 |
| 500 | TrCrCTGGCGGGGAAGT | 54 |
| 483 | THHTGGCGGGGAAGT | 55 |
| 485 | TaCaCTGGCGGGGAAGT | 56 |
| 854 | CTCCTAGCGGGGGCGTCCTAT | 57 |
| | XₐCCTN₂GY_{b}GGGGZ_{c} | 58 |

### DETAILED DESCRIPTION OF THE INVENTION

Definitions

As used herein, the term “antigen” refers to any biological molecule capable of eliciting specific immunity. Antigens specifically include peptides (oligopeptides, polypetides proteins, and glycosylated derivatives thereof), and polysaccharides. Peptide antigens can include preformed peptide antigens and polynucleotides encoding the peptide antigens.

As used herein, the term “autoimmune disease” refers to a disease caused by a breakdown of self-tolerance such that the adaptive immune system responds to self antigens and mediates cell and tissue damage. Autoimmune diseases specifically include, without limitation, insulin-dependent diabetes mellitus, inflammatory bowel disease, and multiple sclerosis. Additional specific examples of autoimmune diseases are provided below.

As used herein, the term “condition associated with CpG-mediated immunostimulation” refers to any disease or other condition in a subject in which there is immune activation associated with exposure of immune cells of the subject to CpG-containing material. Such conditions typically involve activation of TLR9 signaling in response to contact with the CpG.

As used herein, the term “conjugate” refers to any combination of two or more component parts that are linked together, directly or indirectly, via any physicochemical interaction. In one embodiment the conjugate is a combination of two or more component parts that are linked together, directly or indirectly, via covalent bonding.

As used herein, the term “cytidine derivative” refers to a cytidine-like nucleotide (excluding cytidine) having a chemical modification involving the cytosine base, cytidine nucleoside sugar, or both the cytosine base and the cytidine nucleoside sugar. Cytidine derivatives specifically include, without limitation, 5-methylcytidine, 2′-O-methylcytidine, 5-bromocytidine, 5-hydroxycytidine, ribocytidine, and ara-C (cytosine-β-D-arabinofuranoside). Additional specific cytidine derivatives are disclosed further below.

As used herein, the term “effective amount” refers to that amount of a substance that is sufficient to bring about a desired biologic effect. An effective amount can but need not be limited to an amount administered in a single administration.

As used herein, the term “guanosine derivative” refers to a guanosine-like nucleotide (excluding guanosine) having a chemical modification involving the guanine base, guanosine nucleoside sugar, or both the guanine base and the guanosine nucleoside sugar. Guanosine derivatives specifically include, without limitation, 7-deazaguanosine. Additional specific guanosine derivatives are disclosed further below.

As used herein, the term “immune cell that is sensitive to a CpG nucleic acid molecule” refers to a naturally occurring or engineered cell that is activated in response to contact with a CpG nucleic acid molecule. The activation can be manifested in terms of an increase of gene transcription, cell-cycle entry, proliferation, resistance to apoptosis, secretion of a gene product, expression of a gene product, or cytolytic activity. In one embodiment the activation is manifested as an increase in TLR9 signaling.

As used herein, the term “immunoinhibitory nucleic acid molecule” refers to a nucleic acid molecule that is or that includes an inhibitory ODN of the invention.

As used herein, the term “immunostimulatory CpG nucleic acid molecule” refers to any CpG-containing nucleic acid molecule that is capable of activating an immune cell. At least the C of the CpG dinucleotide is typically, but not necessarily, unmethylated. Immunostimulatory CpG nucleic acid molecules are well described in a number of issued patents and published patent applications, including U.S. Pat. Nos. 6,194,388; 6,207,646; 6,218,371; 6,239,116; 6,339,068; 6,406,705; and 6,429,199.

As used herein, the term “immunostimulatory effect of a CpG nucleic acid molecule” refers to any activating or proliferative effect on an immune cell or population of immune cells that is associated with exposure of the immune cell or population of immune cells with a CpG nucleic acid molecule. An activating effect includes increased or de novo expression or secretion of a gene product compared to expression or secretion of that gene product by an immune cell or population of immune cells that has not been exposed to a CpG nucleic acid molecule.

As used herein, the term “infection with a CpG-containing microbe” refers to an abnormal presence of a nucleic acid-containing infectious agent in a host. An infection with a CpG-containing microbe specifically includes a bacterial, viral, fungal, or parasitic infection, and any combination thereof.

As used herein, the term “inflammation” refers to an antigen-nonspecific reaction of the innate immune system that involves accumulation and activation of leukocytes and plasma proteins at a site of infection, toxin exposure, or cell injury. Cytokines that are characteristic of inflammation include tumor necrosis factor (TNF-α), interleukin 1 (IL-1), IL-6, IL-12, interferon alpha (IFN-α), interferon beta (IFN-β), and chemokines.

As used herein, the term “inhibit” shall mean reduce an outcome or effect compared to normal.

As used herein, the term “inhibiting” refers to reducing an outcome or effect compared to normal.

As used herein, the term “isolated” as used to describe a compound shall mean removed from the natural environment in which the compound occurs in nature. In one embodiment isolated means removed from non-nucleic acid molecules of a cell.

As used herein, the term “pharmaceutically acceptable carrier” refers to one or more compatible solid or liquid filler, diluents or encapsulating substances which are suitable for administration to a human or other vertebrate animal.

As used herein, the term “phosphorothioate backbone” refers to a stabilized sugar phosphate backbone of a nucleic acid molecule in which a non-bridging phosphate oxygen is replaced by sulfur at at least one internucleotide linkage. In one embodiment a non-bridging phosphate oxygen is replaced by sulfur at each and every internucleotide linkage.

As used herein, the term “sepsis” refers to a well-recognized clinical syndrome associated with a host's systemic inflammatory response to microbial invasion. Sepsis is typically signaled by fever or hypothermia, tachycardia, and tachypnea, and in severe instances can progress to hypotension, organ dysfunction, and even death.

As used herein, the term “subject” refers to a human or non-human vertebrate. Non-human vertebrates include livestock animals, companion animals, and laboratory animals. Non-human subjects also specifically include non-human primates as well as rodents. Non-human subjects also specifically include, without limitation, chickens, horses, cows, pigs, goats, dogs, cats, guinea pigs, hamsters, mink, and rabbits.

As used herein, the term “subject at risk of developing” a condition refers to a subject with a known or suspected exposure to an agent known to cause or to be associated with the condition or a known or suspected predisposition to develop the condition (e.g., a genetic marker for or a family history of the condition).

As used herein, the term “Th1-like” refers to having a feature characteristic of a Th1 immune response. A Thl immune response characteristically may include induction of certain cytokines such as IFN-γ, secretion (in mice) of IgG2a immunoglobulins, and macrophage activation. The term “Th1-like” is to be contrasted with the term “Th2-like”, which refers to having a feature characteristic of a Th2 immune response. A Th2 immune response characteristically may include induction of certain cytokines such as IL-4 and IL-5, and (in mice) secretion of IgG1 and IgE.

As used herein, the term “TLR signaling” refers to any aspect of intracellular signaling associated with signaling through a TLR.

As used herein, the term “TLR7 signaling agonist” refers to any agent that is capable of inducing an increase in TLR7 signaling. TLR7 signaling agonists specifically include, without limitation, imiquimod (R-837; 1 -(2-methylpropyl)-1 H-imidazo[4,5-c]quinoline-4-amine), resiquimod (R-848; 4-amino-α,α-dimethyl-2-ethoxymethyl-1H-imidazo[4,5-c]quinoline-1 -ethanol), mixtures of ribonucleosides consisting essentially of G and U, or isolated guanosine ribonucleotides such as loxoribine (7-allyl-7,8-dihydro-8-oxo-guanosine; Heil F et al. (2003) Eur J Immunol 33:2987-97) and RNA or RNA-like molecules (PCT/US03/10406).

As used herein, the term “TLR8 signaling agonist” refers to any agent that is capable of inducing an increase in TLR8 signaling. TLR8 signaling agonists specifically include, without limitation, R-837 or R-848 (WO 02/22125), mixtures of ribonucleosides consisting essentially of G and U, and RNA or RNA-like molecules (PCT/US03/10406).

As used herein, the term “TLR9 signaling agonist” refers to any agent that is capable of inducing an increase in TLR9 signaling. TLR9 signaling agonists specifically include, without limitation, immunostimulatory CpG nucleic acid molecules.

As used herein, the term “treat” as used in reference to a disease or condition shall mean to intervene in such disease or condition so as to prevent or slow the development of, prevent or slow the progression of, halt the progression of, or eliminate the disease or condition.

Specific Embodiments

The invention is based in part on the discovery by the applicants that certain oligonucleotides are TLR signaling antagonists. A feature of the invention is the identification of certain oligonucleotide sequence motifs that make it possible to inhibit, selectively, signaling by any one of or by any combination of TLR7, TLR8, and TLR9.

Toll-like receptors (TLRs) are a family of highly conserved polypeptides that play a critical role in innate immunity in mammals. Currently ten family members, designated TLR1-TLR10, have been identified. The cytoplasmic domains of the various TLRs are characterized by a Toll-interleukin 1 (IL-1) receptor (TIR) domain. Medzhitov R et al. (1998) Mol Cell 2:253-8. Recognition of microbial invasion by TLRs triggers activation of a signaling cascade that is evolutionarily conserved in Drosophila and mammals. The TIR domain-containing adapter protein MyD88 has been reported to associate with TLRs and to recruit IL-1 receptor-associated kinase (IRAK) and tumor necrosis factor (TNF) receptor-associated factor 6 (TRAF6) to the TLRs. The MyD88-dependent signaling pathway is believed to lead to activation of NF-κB transcription factors and c-Jun NH₂ terminal kinase (Jnk) mitogen-activated protein kinases (MAPKs), critical steps in immune activation and production of inflammatory cytokines. For a review, see Aderem A et al. (2000) Nature 406:782-87.

While a number of specific TLR ligands have been reported, ligands for some TLRs remain to be identified. Ligands for TLR2 include peptidoglycan and lipopeptides. Yoshimura A et al. (1999) J Immunol 163:1-5; Yoshimura A et al. (1999) J Immunol 163:1-5; Aliprantis AO et al. (1999) Science 285:736-9. Viral-derived double-stranded RNA (dsRNA) and poly I:C, a synthetic analog of dsRNA, have been reported to be ligands of TLR3. Alexopoulou L et al. (2001) Nature 413:732-8. Lipopolysaccharide (LPS) is a ligand for TLR4. Poltorak A et al. (1998) Science 282:2085-8; Hoshino K et al. (1999) J Immunol 162:3749-52. Bacterial flagellin is a ligand for TLR5. Hayashi F et al. (2001) Nature 410:1099-1103. Peptidoglycan has been reported to be a ligand not only for TLR2 but also for TLR6. Ozinsky A et al. (2000) Proc Natl Acad Sci USA 97:13766-71; Takeuchi O et al. (2001) Int Immunol 13:933-40. Certain low molecular weight synthetic compounds, the imidazoquinolones imiquimod (R-837) and resiquimod (R-848), were reported to be ligands of TLR7 and TLR8. Hemmi H et al. (2002) Nat Immunol 3:196-200; Jurk M et al. (2002) Nat Immunol 3:499. Bacterial DNA (CpG DNA) has been reported to be a TLR9 ligand. Hemmi H et al. (2000) Nature 408:740-5; Bauer S et al. (2001) Proc Natl Acad Sci USA 98, 9237-42. RNA molecules were recently identified that are believed to interact with and signal through TLR7 and TLR8. PCT/US03/10406.

In addition to having diverse ligands, the various TLRs are believed to be differentially expressed in various tissues and on various types of immune cells. For example, human TLR7 has been reported to be expressed in placenta, lung, spleen, lymph nodes, tonsil and on plasmacytoid precursor dendritic cells (pDCs). Chuang T-H et al. (2000) Eur Cytokine Netw 11:372-8); Kadowaki N et al. (2001) J Exp Med 194:863-9. Human TLR8 has been reported to be expressed in lung, peripheral blood leukocytes (PBL), placenta, spleen, lymph nodes, and on monocytes. Kadowaki N et al. (2001) J Exp Med 194:863-9; Chuang T-H et al. (2000) Eur Cytokine Netw 11:372-8. Human TLR9 is reportedly expressed in spleen, lymph nodes, bone marrow, PBL, and on pDCs, and B cells. Kadowaki N et al. (2001) J Exp Med 194:863-9; Bauer S et al. (2001) Proc Natl Acad Sci USA 98:9237-42; Chuang T-H et al. (2000) Eur Cytokine Netw 11:372-8.

Nucleotide and amino acid sequences of human and murine TLR7 are known. See, for example, GenBank Accession Nos. AF240467, AF245702, NM_{—}016562, AF334942, NM_{—}133211; and AAF60188, AAF78035, NP_{—}057646, AAL73191, and AAL73192, the contents of all of which are incorporated herein by reference. Human TLR7 is reported to be 1049 amino acids long. Murine TLR7 is reported to be 1050 amino acids long. TLR7 polypeptides include an extracellular domain having a leucine-rich repeat region, a transmembrane domain, and an intracellular domain that includes a TIR domain.

Nucleotide and amino acid sequences of human and murine TLR8 are known. See, for example, GenBank Accession Nos. AF246971, AF245703, NM_{—}016610, XM_{—}045706, AY035890, NM_{—}133212; and AAF64061, AAF78036, NP_{—}057694, XP_{—}045706, AAK62677, and NP_{—}573475, the contents of all of which is incorporated herein by reference. Human TLR8 is reported to exist in at least two isoforms, one 1041 amino acids long and the other 1059 amino acids long. Murine TLR8 is 1032 amino acids long. TLR8 polypeptides include an extracellular domain having a leucine-rich repeat region, a transmembrane domain, and an intracellular domain that includes a TIR domain.

Nucleotide and amino acid sequences of human and murine TLR9 are known. See, for example, GenBank Accession Nos. NM_{—}017442, AF259262, AB045180, AF245704, AB045181, AF348140, AF314224, NM_{—}031178; and NP_{—}059138, AAF72189, BAB19259, AAF78037, BAB19260, AAK29625, AAK28488, and NP_{—}112455, the contents of all of which are incorporated herein by reference. Human TLR9 is reported to exist in at least two isoforms, one 1032 amino acids long and the other 1055 amino acids. Murine TLR9 is 1032 amino acids long. TLR9 polypeptides include an extracellular domain having a leucine-rich repeat region, a transmembrane domain, and an intracellular domain that includes a TIR domain.

TLR9-Antagonist Motif

In some aspects the invention provides novel inhibitory ODN characterized at least in part by a TLR9-antagonist motif. The TLR9-antagonist motif includes a 5∝ CC dinucleotide, wherein the cytidines of the 5′ CC dinucleotide can be cytidine derivatives, followed by a G-rich sequence. It was not previously recognized how important the 5′ CC dinucleotide is to the inhibitory effect of inhibitory ODN. Neither was it previously recognized that the cytidines of the 5′ CC dinucleotide can be cytidine derivatives.

In one aspect the invention provides a composition including an isolated immunoinhibitory nucleic acid molecule including a sequence
XₐCCN₁N₂N₃Y_{b}N₄GGGZ_{c} (SEQ ID NO:1)
   wherein each C is cytidine or a derivative thereof, wherein at least one C is a cytidine derivative; each G is guanosine or a deaza derivative thereof; Xₐ is any nucleotide sequence a nucleotides long, wherein a is an integer between 0-12, inclusive, and each nucleotide is selected independently of any other in Xₐ; Y_{b} is any nucleotide sequence b nucleotides long, wherein b is an integer between 0-21, inclusive, and each nucleotide is selected independently of any other in Y_{b}; Z_{c}is any nucleotide sequence c nucleotides long, wherein c is an integer between 0-12, inclusive, and each nucleotide is selected independently of any other in Z_{c}; and N₁, N₂, N₃, and N₄ are each independently any nucleotide. It will be appreciated that the inhibitory ODN according to this aspect of the invention includes at least four nucleotides between the 5′ CC dinucleotide and a triplet GGG. In view of the ranges for each of Xₐ, Y_{b}, and Z_{c}, inhibitory ODN according to this aspect of the invention can range between 9 and 54 nucleotides long.

Featured according to this aspect of the invention is the inclusion of one or two cytidine derivatives in the 5′ CC dinucleotide. In one embodiment both C's of the 5′ CC dinucleotide are identically selected cytidine derivatives. Cytidine derivatives generally will include, without limitation, cytidines with modified cytosine base. Modified cytosines include but are not limited to 5-substituted cytosines (e.g., 5-(C₁-C₆)-alkylcytosine, 5-(C₂-C₆)-alkenylcytosine, 5-(C₂-C₆)-alkynylcytosine, 5-methyl-cytosine, 5-fluoro-cytosine, 5-chloro-cytosine, 5-bromo-cytosine, 5-iodo-cytosine, 5-hydroxy-cytosine, 5-hydroxymethyl-cytosine, 5-difluoromethyl-cytosine, and unsubstituted or substituted 5-alkynyl-cytosine), 6-substituted cytosines, N4-substituted cytosines (e.g., N4-alkylcytosine, including N4-ethyl-cytosine), 5-aza-cytosine, 2-mercapto-cytosine, isocytosine, pseudo-isocytosine, cytosine analogs with condensed ring systems (e.g., N,N′-propylene cytosine or phenoxazine), and uracil and its derivatives (e.g., 5-fluoro-uracil, 5-bromo-uracil, 5-bromovinyl-uracil, 4-thio-uracil, 5-hydroxy-uracil, 5-propynyl-uracil). Cytidine derivatives generally will also include, without limitation, cytidines with modified sugars. Cytidines with modified sugars include but are not limited to cytosine-β-D-arabinofuranoside (Ara-C), ribo-C, and 2′-O-(C₁-C₆)alkyl-cytidine (e.g., 2′-O-methylcytidine, 2′-OMe-C).

The insensitivity to substitution of a cytidine derivative for either or both C's of the 5′ CC dinucleotide in inhibitory ODN is to be contrasted to the generally profound sensitivity to substitution of a methylated C for the unmethylated C of the CpG dinucleotide in immune stimulatory CpG nucleic acids. As described in Example 5 below, derivatives of inhibitory ODN 2088 generally retained their inhibitory effect despite substitution of both C's of the 5′ CC dinucleotide with 2′-OMe-C, 5-methyl-C, 5 bromo-C, ribo-C, 5-hydroxy-C, or Ara-C.

As described in Example 3 below, even though the C's of the 5′ CC dinucleotide can be replaced with cytidine derivatives, deletion of one of the C's or substitution of the CC dinucleotide with any other dinucleotide, e.g., with AT, surprisingly largely abolishes the inhibitory effect.

As the formula above suggests, the 5′ CC dinucleotide and the triplet GGG are separated by intervening structure including a minimum of four nucleotides or nucleotide analogs. Intervening sequence can include, inter alia, further C and G nucleotides. In one embodiment N₄ is G or guanosine derivative, thereby extending the triplet GGG to a G quartet. G quartets have been described in the literature to be involved in forming multimeric nucleic acid structures, presumably through stacking interactions. Surprisingly, however, the inhibitory effect of inhibitory ODN is not affected by replacement of any of the Gs with a guanosine derivative, such as a deazaguanosine, that prevents planar stacking. Thus according to this aspect of the invention, in one embodiment the inhibitory ODN includes both a cytidine derivative for either or both C's of the 5′ CC dinucleotide and at least one guanosine derivative, such as a deazaguanosine, that prevents planar stacking. In one embodiment the at least one guanosine derivative is 7-deazaguanosine.

It will be noted that Xₐ, N₁, N₂, N₃, Y_{b}, N₄, Z_{c}, or a combination thereof can include sequence such that the CC and GGG motifs are not unique. Accordingly, in certain embodiments the CC and GGG motifs are chosen such that b is the smallest integer between 0-21, inclusive, able to conform to the sequence XₐCCN₁N₂N₃Y_{b}N₄GGGZ_{c} according to this aspect of the invention. In certain embodiments the CC and GGG motifs are chosen such that b is the largest integer between 0-21, inclusive, able to conform to the sequence XₐCCN₁N₂N₃Y_{b}N₄GGGZ_{c} according to this aspect of the invention.

Specific examples of inhibitory ODN according to this aspect of the invention include, without limitation,
TCCTGGCGGGGAAGT (SEQ ID NO:4),
GCCTGGCGGGGAAGT (SEQ ID NO:5),
ACCTGGCGGGGAAGT (SEQ ID NO:6),
CCCTGGCGGGGAAGT (SEQ ID NO:7),
TCCCGGCGGGGAAGT (SEQ ID NO:8),
TCCGGGCGGGGAAGT (SEQ ID NO:9),
TCCTAGCGGGGAAGT (SEQ ID NO:10),
TCCTGGAGGGGAAGT (SEQ ID NO:11),
TCCTAGCGGGGGCGTCCTAT (SEQ ID NO:12), and
CCTCAAGCTTGAGGGG (SEQ ID NO:13).

In one aspect the invention provides an isolated immunoinhibitory nucleic acid molecule including a sequence
XₐCCN₁N₂N₃Y_{b}N₄GGGZ_{c}(SEQ ID NO:1)
   wherein each C is independently cytidine or a derivative thereof; each G is independently guanosine or a deaza derivative thereof; Xₐ is any nucleotide sequence a nucleotides long, wherein a is an integer between 0-12, inclusive, and each nucleotide is selected independently of any other in Xₐ; Y_{b} is any nucleotide sequence b nucleotides long, wherein b is an integer between 8-21, inclusive, and each nucleotide is selected independently of any other in Y_{b}; Z_{c} is any nucleotide sequence c nucleotides long, wherein c is an integer between 0-12, inclusive, and each nucleotide is selected independently of any other in Z_{c}; and N₁, N₂, N₃, and N₄ are each independently any nucleotide.

Featured according to this aspect of the invention is the inclusion of at least twelve nucleotides between a 5′ CC dinucleotide and a triplet GGG. In view of the ranges for each Of Xₐ, Y_{b}, and Z_{c}, inhibitory ODN according to this aspect of the invention can range between 17 and 54 nucleotides long.

In one embodiment according to this aspect of the invention, both C's of the 5′ CC dinucleotide of the inhibitory ODN are cytidines, i.e., neither C of the 5′ CC dinucleotide is a cytidine derivative. In other embodiments, either or both C's of the 5′ CC dinucleotide of the inhibitory ODN are cytidine derivatives, as described above.

It will be noted that Xₐ, N₁, N₂, N₃, Y_{b}, N₄, Z_{c}, or a combination thereof can include sequence such that the CC and GGG motifs are not unique. Accordingly, in certain embodiments the CC and GGG motifs are chosen such that b is the smallest integer between 8-21, inclusive, able to conform to the sequence XₐCCN₁N₂N₃Y_{bN}₄GGGZ_{c}according to this aspect of the invention. In certain embodiments the CC and GGG motifs are chosen such that b is the largest integer between 8-21, inclusive, able to conform to the sequence XₐCCN₁N₂N₃Y_{b}N₄GGGZ_{c}according to this aspect of the invention.

Specific examples of inhibitory ODN according to this aspect of the invention include, without limitation, TCCTGTGTGTGTGTCGGGGAAGT (SEQ ID NO:14), TCCTGTGTGTGTGTGTCGGGGAAGT (SEQ ID NO:15), and TCCTGTGTGTGTGTGTGTCGGGGAAGT (SEQ ID NO:16).

In one aspect the invention provides an isolated immunoinhibitory nucleic acid molecule including a sequence
XₐCCN₁Y_{b}N₂N₃N₄N₅GGZ_{c} (SEQ ID NO:2)
   wherein N₂N₃N₄N₅GG is selected from GGN₄N₅GG, GN₃N₄GGG, N₂GN₄GGG, and N₂N₃N₄GGG; each C is cytidine or a derivative thereof; each G is guanosine or a deaza derivative thereof; Xₐ is any nucleotide sequence a nucleotides long, wherein a is an integer between 0-12, inclusive, and each nucleotide is selected independently of any other in Xₐ; Y_{b} is any nucleotide sequence b nucleotides long, wherein b is an integer between 8-21, inclusive, and each nucleotide is selected independently of any other in Y_{b}; Z_{c}is any nucleotide sequence c nucleotides long, wherein c is an integer between 0-12, inclusive, and each nucleotide is selected independently of any other in Z_{c}; and N₁, N₂, N₃, N₄, and N₅ are each independently any nucleotide. It will be appreciated that the inhibitory ODN according to this aspect of the invention includes at least thirteen nucleotides between a 5′ CC dinucleotide and a GG dinucleotide. In view of the ranges for each of Xₐ, Y_{b}, and Z_{c}, inhibitory ODN according to this aspect of the invention can range between 17 and 48 nucleotides long.

Featured according to this aspect of the invention is the inclusion of at least thirteen nucleotides between a 5′ CC dinucleotide and a GG dinucleotide, wherein the at least thirteen intervening nucleotides include GGN₄Ns, GN₃N₄G, N₂GN₄G, or N₂N₃N₄G immediately 5′ to the GG dinucleotide, thereby calling for a GGN₄N₅GG, GN₃N₄GGG, N₂GN₄GGG, or N₂N₃N₄GGG motif appropriately spaced downstream of the 5′ CC dinucleotide.

In one embodiment according to this aspect of the invention, both C's of the 5′ CC dinucleotide of the inhibitory ODN are cytidines, i.e., neither C of the 5′ CC dinucleotide is a cytidine derivative. In other embodiments, either or both C's of the 5′ CC dinucleotide of the inhibitory ODN are cytidine derivatives, as described above.

It will be noted that Xₐ, N₁, N₂, N₃, Y_{b}, N₄, Z_{c}, or a combination thereof can include sequence such that the CC and GG motifs are not unique. Accordingly, in certain embodiments the CC and GG motifs are chosen such that b is the smallest integer between 8-21, inclusive, able to conform to the sequence XₐCCN₁Y_{b}N₂N₃N₄NSGGZ_{c} according to this aspect of the invention. In certain embodiments the CC and GG motifs are chosen such that b is the largest integer between 8-21, inclusive, able to conform to the sequence XₐCCN₁Y_{b}N₂N₃N₄N₅GGZ_{c}according to this aspect of the invention.

Specific examples of inhibitory ODN according to this aspect of the invention include, without limitation, TCCTGTGTGTGTGTCGGGGAAGT (SEQ ID NO:14), TCCTGTGTGTGTGTGTCGGGGAAGT (SEQ ID NO:1 5), and TCCTGTGTGTGTGTGTGTCGGGGAAGT (SEQ ID NO:16).

TLR8-Antagonist Motif

In some aspects the invention provides novel inhibitory ODN characterized at least in part by a TLR8-antagonist motif. The TLR8-antagonist motif includes an oligonucleotide 2-100 nucleotides long having a 3′ end terminating with the dinucleotide GK, wherein G is guanosine or 7-deazaguanosine and K is T (thymidine), U (uracil), or G. In one embodiment the TLR8-antagonist motif includes an oligonucleotide 2 to 40 nucleotides long having a 3′ end terminating with the dinucleotide GK. In one embodiment the TLR8-antagonist motif includes an oligonucleotide 2 to about 30 nucleotides long having a 3′ end terminating with the dinucleotide GK.

As described in Example 7 below, it was surprisingly found that the simple dinucleotide GT by itself exerts a significant inhibitory effect on TLR8 signaling. As also described in Example 7 below, it was surprisingly found that the simple dinucleotide GU by itself exerts a significant inhibitory effect on TLR8 signaling.

Also as described in Example 7 below, relocating the GK dinucleotide to the 5′ end or to the interior of a longer oligonucleotide (e.g., a 1 5-mer), results in essentially a complete loss of the inhibitory effect on TLR8. In one embodiment any one or more nucleotides upstream of the 3′ terminal dinucleotide GK can be replaced with dSpacer. However, stretches of repeated nucleotides (e.g., >4 successive T or >4 successive A) was found to reduce inhibitory capacity of the ODN significantly. In one embodiment the inhibitory ODN including the TLR8-antagonist motif has a phosphorothioate backbone. In one embodiment the inhibitory ODN including the TLR8-antagonist motif has a sugar phosphate backbone including at least one 2′-OMe sugar.

It will be noted that certain embodiments of the inhibitory ODN having a TLR9-antagonist motif, described above, include a 3′ end terminating with the dinucleotide GK as just described with reference to inhibitory ODN having a TLR8-antagonist motif. Examples of such inhibitory ODN include, without limitation, TCCTGGCGGGGAAGT (SEQ ID NO:4), GCCTGGCGGGGAAGT (SEQ ID NO:5), ACCTGGCGGGGAAGT (SEQ ID NO:6), CCCTGGCGGGGAAGT (SEQ ID NO:7), TCCCGGCGGGGAAGT (SEQ ID NO:8), TCCGGGCGGGGAAGT (SEQ ID NO:9), TCCTAGCGGGGAAGT (SEQ ID NO:10), TCCTGGAGGGGAAGT (SEQ ID NO:11), TCCTGTGTGTGTGTGTGTCGGGGAAGT (ODN 497; SEQ ID NO:16), TZZTGGCGGGGAAGT (ODN 159; Z=5-Methyl-C; SEQ ID NO:32), and TCCTGGCGGGGAAGT (ODN 166; C=2′-O-methylcytidine; SEQ ID NO:33). It has been found according to the present invention that such inhibitory ODN are in fact inhibitory for both TLR9 and TLR8.

It should also be noted that, certain embodiments of the inhibitory ODN having a TLR9-antagonist motif, described above, do not include a 3′ end terminating with the dinucleotide GK as described with reference to inhibitory ODN having a TLR8-antagonist motif. Examples of such inhibitory ODN include, without limitation, TCCTAGCGGGGGCGTCCTAT (SEQ ID NO:12), and CCTCAAGCTTGAGGGG (SEQ ID NO:13). It has been found according to the present invention that such inhibitory ODN are in fact inhibitory for TLR9 but not for TLR8.

It should further be noted that, certain embodiments of the inhibitory ODN having a 3′ end terminating with the dinucleotide GK as described with reference to inhibitory ODN having a TLR8-antagonist motif, described above, do not include a TLR9-antagonist motif. Examples of such inhibitory ODN include, without limitation, TGCTGGCGGGGAAGT (ODN 443; SEQ ID NO:34), TGATGGCGGGGAAGT (ODN 444; SEQ ID NO:35), and TGTTGGCGGGGAAGT (ODN 445; SEQ ID NO:36). It has been found according to the present invention that such inhibitory ODN are in fact inhibitory for TLR8 but not for TLR9.

TLR7-Antagonist Motif

In some aspects the invention provides novel inhibitory ODN characterized at least in part by a TLR7-antagonist motif. The TLR7-antagonist motif includes any nucleotide sequence 6-100 nucleotides long having a phosphorothioate backbone. In some embodiments the TLR7 antagonist motif is present within an oligonucleotide having a partially phosphorothioate backbone. In these embodiments, the oligonucleotide is 10 to 100 nucleotides long and includes a backbone in which less than 25 percent of linkages are consecutive phosphodiester linkages. For example, it has been found according to the invention that a chimeric phosphodiester/phosphorothioate 15-mer with 3 consecutive phosphodiester linkages effectively inhibited TLR7 signaling, while a chimeric phosphodiester/phosphorothioate 15-mer with 6 consecutive phosphodiester linkages did not effectively inhibit TLR7 signaling. It is believed that the percentage of phosphodiester character may be less stringent if the phosphodiester linkages are nonconsecutive.

In some embodiments the inhibitory ODN characterized at least in part by a TLR7-antagonist motif also includes a TLR9-inhibitory motif but not a TLR8-inhibitory motif. Such inhibitory ODN have been found according to the invention to inhibit TLR7 and TLR9 but not TLR8. For example, in one embodiment the inhibitory ODN is an isolated immunoinhibitory nucleic acid molecule including a sequence XₐCCN₁N₂N₃Y_{b}N₄GGGZ_{c} (SEQ ID NO:1) or XₐCCN₁Y_{b}N₂N₃N₄N₅GGZ_{c} (SEQ ID NO:2), each as described above, wherein Z_{c}is not K when c is 1 and wherein Z_{c}does not terminate with GK when c is an integer between 2-12, inclusive, wherein G is chosen from guanosine and 7-deazaguanosine and K is chosen from thymidine (T), uracil (U), and guanosine. In one embodiment the inhibitory ODN is an isolated immunoinhibitory nucleic acid molecule including a sequence XₐCCN₁N₂N₃Y_{b}N₄GGGZ_{c} (SEQ ID NO:1) or XₐCCN₁Y_{b}N₂N₃N₄N₅GGZ_{c} (SEQ ID NO:2), each as described above, wherein Z_{c}is not T when c is 1 and wherein Z_{c}does not terminate with GT when c is an integer between 2-12, inclusive. In one embodiment Z_{c}is chosen from A, C, G, or a derivative thereof when c is 1. In one embodiment Z_{c}terminates with a dinucleotide chosen from AA, AC, AG, AT, AU, CA, CC, CG, CT, CU, GA, GC, TA, TC, TG, TT, TU, UA, UC, UG, UT, or UU when c is an integer between 2-12, inclusive.

In some embodiments the inhibitory ODN characterized at least in part by a TLR7-antagonist motif also includes a TLR8-inhibitory motif but not a TLR9-inhibitory motif. Such oligonucleotides have been found according to the invention to inhibit TLR7 and TLR8 but not TLR9.

In some embodiments the inhibitory ODN characterized at least in part by a TLR7-antagonist motif also includes both a TLR9-inhibitory motif and a TLR8-inhibitory motif. Such oligonucleotides have been found according to the invention to inhibit TLR7 and TLR8 and TLR9. For example, in one embodiment the inhibitory ODN is an isolated immunoinhibitory nucleic acid molecule including a sequence XₐCCN₁N₂N₃Y_{b}N₄GGGZ_{c} (SEQ ID NO:1) or XₐCCN₁Y_{b}N₂N₃N₄N₅GGZ_{c} (SEQ ID NO:2), each as described above, wherein Z_{c}is T when c is 1 and wherein Z_{c}terminates with GT when c is an integer between 2-12, inclusive. As another example, in one embodiment the inhibitory ODN is an isolated immunoinhibitory nucleic acid molecule including a sequence XₐCCN₁N₂N₃Y_{b}N₄GGGZ_{c} (SEQ ID NO:1) or XₐCCN₁Y_{b}N₂N₃N₄N₅GGZ_{c}(SEQ ID NO:2), each as described above, wherein Z_{c}is T or U when c is 1 and wherein Z_{c} terminates with 7T or 7U, wherein 7 is 7-deazaguanosine, when c is an integer between 2-12, inclusive.

In some embodiments the inhibitory ODN characterized at least in part by a TLR7-antagonist motif excludes both a TLR9-inhibitory motif and a TLR8-inhibitory motif. Such oligonucleotides have been found according to the invention to inhibit TLR7 but not TLR8 and not TLR9.

Measuring Inhibitory Effects

The inhibitory effect of the inhibitory ODN of the invention can be measured in vitro or in vivo. A basis for such measurement can involve, for example, comparison between stimulation of immune cells contacted with an appropriate source of TLR agonist, in the presence or absence of an appropriate source of inhibitory ODN. Stimulation that is reduced with inhibitory ODN compared with that without inhibitory ODN indicates an inhibitory effect of the inhibitory ODN. The inhibitory effect can be quantified and, if desired, used as the basis for screening or comparing candidate inhibitory ODN. Such screening and comparison can optionally be performed on a high throughput basis.

In one embodiment, a basis for measurement of the inhibitory effect of the inhibitory ODN of the invention in vitro can involve comparison between stimulation of TLR9-expressing cells contacted with an appropriate source of immunostimulatory CpG DNA, in the presence or absence of an appropriate source of inhibitory ODN. The TLR9-expressing cells can be cells that express TLR9 naturally, e.g., B cells or peripheral blood mononuclear cells (PBMC), or they can be cells that express TLR9 artificially, e.g., through transfection with a polynucleotide that encodes a TLR9.

Readouts for such measurements can be any suitable readout for assessing an effect, including TLR9 signaling, associated with immunostimulatory CpG DNA. For example, comparison can be made between B-cell apoptosis, cell cycle entry, cytokine secretion (e.g., IFN-α, IL-6, IL-12, TNF-α, IFN-γ, IP-10), CTL activity, or IgG2a. General methods for performing such measurements are well known in the art and include, for example, cell sorting, cytokine-specific enzyme-linked immunosorbent assay (ELISA), and chromium release cell lysis assay. As described in the Examples, the readout for such measurements can involve measurement of a marker, artificially introduced into a cell, for TLR9 signaling. In one embodiment the marker for TLR9 activity is expression of a gene placed under control of an NF-κB promoter, e.g., NF-κB-luciferase.

In one embodiment, a basis for measurement of the inhibitory effect of the inhibitory ODN of the invention in vitro can involve comparison between stimulation of TLR8-expressing cells contacted with an appropriate source of TLR8 agonist, such as R-848, in the presence or absence of an appropriate source of inhibitory ODN. The TLR8-expressing cells can be cells that express TLR8 naturally, e.g., monocytes or PBMC, or they can be cells that express TLR8 artificially, e.g., through transfection with a polynucleotide that encodes a TLR8.

Readouts for such measurements can be any suitable readout for assessing an effect, including TLR8 signaling, associated with a TLR8 agonist. As described in the Examples, the readout for such measurements can involve measurement of a marker, artificially introduced into a cell, for TLR8 signaling. In one embodiment the marker for TLR8 activity is expression of a gene placed under control of an NF-κB promoter, e.g., NF-κB-luciferase.

In one embodiment, a basis for measurement of the inhibitory effect of the inhibitory ODN of the invention in vitro can involve comparison between stimulation of TLR7-expressing cells contacted with an appropriate source of TLR7 agonist, such as R-848, in the presence or absence of an appropriate source of inhibitory ODN. The TLR7-expressing cells can be cells that express TLR7 naturally, e.g., B cells or PBMC, or they can be cells that express TLR7 artificially, e.g., through transfection with a polynucleotide that encodes a TLR7.

Readouts for such measurements can be any suitable readout for assessing an effect, including TLR7 signaling, associated with a TLR7 agonist. As described in the Examples, the readout for such measurements can involve measurement of a marker, artificially introduced into a cell, for TLR7 signaling. In one embodiment the marker for TLR7 activity is expression of a gene placed under control of an NF-κB promoter, e.g., NF-κB-luciferase.

In each of the foregoing aspects of the invention, the inhibitory ODN has a backbone that may be stabilized. In one embodiment the backbone is a sugar phosphate backbone that includes at least one phosphorothioate internucleotide linkage. In one embodiment the backbone is completely phosphorothioate.

Source and Preparation of Inhibitory ODN of the Invention

The inhibitory ODN of the instant invention can encompass various chemical modifications and substitutions, in comparison to natural RNA and DNA, involving a phosphodiester internucleoside bridge, a β-D-ribose unit and/or a natural nucleoside base (adenine, guanine, cytosine, thymine, uracil). Examples of chemical modifications are known to the skilled person and are described, for example, in Uhlmann E et al. (1990) Chem Rev 90:543; “Protocols for Oligonucleotides and Analogs” Synthesis and Properties & Synthesis and Analytical Techniques, S. Agrawal, Ed, Humana Press, Totowa, USA 1993; Crooke ST et al. (1996) Annu Rev Pharmacol Toxicol 36:107-29; and Hunziker J et al. (1995) Mod Synth Methods 7:331-417. An oligonucleotide according to the invention may have one or more modifications, wherein each modification is located at a particular phosphodiester intemucleoside bridge and/or at a particular β-D-ribose unit and/or at a particular natural nucleoside base position in comparison to an oligonucleotide of the same sequence which is composed of natural DNA or RNA.

For example, the oligonucleotides may include one or more modifications and wherein each modification is independently selected from:
a) the replacement of a phosphodiester intemucleoside bridge located at the 3′ and/or the 5′ end of a nucleoside by a modified intemucleoside bridge,
b) the replacement of phosphodiester bridge located at the 3′ and/or the 5′ end of a nucleoside by a dephospho bridge,
c) the replacement of a sugar phosphate unit from the sugar phosphate backbone by another unit,
d) the replacement of a β-D-ribose unit by a modified sugar unit, and
e) the replacement of a natural nucleoside base by a modified nucleoside base.

More detailed examples for the chemical modification of an oligonucleotide are as follows.

The oligonucleotides may include modified internucleotide linkages, such as those described in a or b above. These modified linkages may be partially resistant to degradation (e.g., are stabilized). A “stabilized oligonucleotide molecule” shall mean an oligonucleotide that is relatively resistant to in vivo degradation (e.g., via an exo- or endo-nuclease) resulting from such modifications. Oligonucleotides having phosphorothioate linkages, in some embodiments, may provide maximal activity and protect the oligonucleotide from degradation by intracellular exo- and endo-nucleases.

A phosphodiester internucleoside bridge located at the 3′ and/or the 5′ end of a nucleoside can be replaced by a modified internucleoside bridge, wherein the modified internucleoside bridge is for example selected from phosphorothioate, phosphorodithioate, NR¹R²-phosphoramidate, boranophosphate, α-hydroxybenzyl phosphonate, phosphate-(C₁,-C₂₁)-O-alkyl ester, phosphate-[(C₆-C₁₂)aryl-(C₁-C₂₁)-O-alkyl]ester, (C₁-C₈)alkylphosphonate and/or (C₆-C₁₂)arylphosphonate bridges, (C₇-C₁₂)-α-hydroxymethyl-aryl (e.g., disclosed in WO 95/01363), wherein (C₆-C₁₂)aryl, (C₆-C₂₀)aryl and (C₆-C₁₄)aryl are optionally substituted by halogen, alkyl, alkoxy, nitro, cyano, and where R¹ and R² are, independently of each other, hydrogen, (C₁-C₁₈)-alkyl, (C₆-C₂₀)-aryl, (C₆-C₁₄)-aryl-(C₁-C₈)-alkyl, preferably hydrogen, (C₁-C₈)-alkyl, preferably (C₁-C4)-alkyl and/or methoxyethyl, or R¹ and R² form, together with the nitrogen atom carrying them, a 5-6-membered heterocyclic ring which can additionally contain a further heteroatom from the group O, S and N.

The replacement of a phosphodiester bridge located at the 3′ and/or the 5′ end of a nucleoside by a dephospho bridge (dephospho bridges are described, for example, in Uhlmann E and Peyman A in “Methods in Molecular Biology”, Vol. 20, “Protocols for Oligonucleotides and Analogs”, S. Agrawal, Ed., Humana Press, Totowa 1993, Chapter 16, pp. 355 ff), wherein a dephospho bridge is for example selected from the dephospho bridges formacetal, 3′-thioformacetal, methylhydroxylamine, oxime, methylenedimethyl-hydrazo, dimethylenesulfone and/or silyl groups.

A sugar phosphate unit (i.e., a β-D-ribose and phosphodiester intemucleoside bridge together forming a sugar phosphate unit) from the sugar phosphate backbone (i.e., a sugar phosphate backbone is composed of sugar phosphate units) can be replaced by another unit, wherein the other unit is for example suitable to build up a “morpholino-derivative” oligomer (as described, for example, in Stirchak EP et al. (1989) Nucleic Acids Res 17:6129-41), that is, e.g., the replacement by a morpholino-derivative unit; or to build up a polyamide nucleic acid (“PNA”; as described for example, in Nielsen PE et al. (1994) Bioconjug Chem 5:3-7), that is, e.g., the replacement by a PNA backbone unit, e.g., by 2-aminoethylglycine. The oligonucleotide may have other carbohydrate backbone modifications and replacements, such as peptide nucleic acids with phosphate groups (PHONA), locked nucleic acids (LNA), and oligonucleotides having backbone sections with alkyl linkers or amino linkers. The alkyl linker may be branched or unbranched, substituted or unsubstituted, and chirally pure or a racemic mixture.

A β-ribose unit or a β-D-2′-deoxyribose unit can be replaced by a modified sugar unit, wherein the modified sugar unit is for example selected from β-D-ribose, α-D-2′-deoxyribose, L-2′-deoxyribose, 2′-F-2′-deoxyribose, 2′-F-arabinose, 2′-O-(C₁-C₆)alkyl-ribose, 2′-O-methylribose, 2′-O-(C₂-C₆)alkenyl-ribose, 2′-[O-(C₁-C₆)alkyl-O-(C₁-C₆)alkyl]-ribose, 2′-NH₂-2′-deoxyribose, β-D-xylo-furanose, α-arabinofuranose, 2,4-dideoxy-β-D-erythro-hexo-pyranose, and carbocyclic (described, for example, in Froehler (1992) J Am Chem Soc 114:8320) and/or open-chain sugar analogs (described, for example, in Vandendriessche et al. (1993) Tetrahedron 49:7223) and/or bicyclosugar analogs (described, for example, in Tarkov M et al. (1993) Helv Chim Acta 76:481).

In some embodiments the sugar is 2′-O-methylribose, particularly for one or both nucleotides linked by a phosphodiester or phosphodiester-like internucleoside linkage.

Nucleic acids also include substituted purines and pyrimidines such as C-5 propyne pyrimidine and 7-deaza-7-substituted purine modified bases. Wagner RW et al. (1996) Nat Biotechnol 14:840-4. Purines and pyrimidines include but are not limited to adenine, cytosine, guanine, and thymine, and other naturally and non-naturally occurring nucleobases, substituted and unsubstituted aromatic moieties.

A modified base is any base which is chemically distinct from the naturally occurring bases typically found in DNA and RNA such as T, C, G, A, and U, but which share basic chemical structures with these naturally occurring bases. The modified nucleoside base may be, for example, selected from hypoxanthine, uracil, dihydrouracil, pseudouracil, 2-thiouracil, 4-thiouracil, 5-aminouracil, 5-(C₁-C₆)-alkyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxycytosine, 5-(C₁-C₆)-alkylcytosine, 5-(C₂-C₆)-alkenylcytosine, 5-(C₂-C₆)-alkynylcytosine, 5-chlorocytosine, 5-fluorocytosine, 5-bromocytosine, N²-dimethylguanine, 2,4-diamino-purine, 8-azapurine, a substituted 7-deazapurine, preferably 7-deaza-7-substituted and/or 7-deaza-8-substituted purine, 5-hydroxymethylcytosine, N4-alkylcytosine, e.g., N4-ethylcytosine, 5-hydroxydeoxycytidine, 5-hydroxymethyldeoxycytidine, N4-alkyldeoxycytidine, e.g., N4-ethyldeoxycytidine, 6-thiodeoxyguanosine, and deoxyribonucleosides of nitropyrrole, C5-propynylpyrimidine, and diaminopurine e.g., 2,6-diaminopurine, inosine, 5-methylcytosine, 2-aminopurine, 2-amino-6-chloropurine, hypoxanthine or other modifications of a natural nucleoside bases. This list is meant to be exemplary and is not to be interpreted to be limiting.

In particular formulas described herein modified bases may be incorporated. For instance a cytosine may be replaced with a modified cytosine. A modified cytosine as used herein is a naturally occurring or non-naturally occurring pyrimidine base analog of cytosine which can replace this base without impairing the immunostimulatory activity of the oligonucleotide. Modified cytosines include but are not limited to 5-substituted cytosines (e.g., 5-methyl-cytosine, 5-fluoro-cytosine, 5-chloro-cytosine, 5-bromo-cytosine, 5-iodo-cytosine, 5-hydroxy-cytosine, 5-hydroxymethyl-cytosine, 5-difluoromethyl-cytosine, and unsubstituted or substituted 5-alkynyl-cytosine), 6-substituted cytosines, N4-substituted cytosines (e.g., N4-ethyl-cytosine), 5-aza-cytosine, 2-mercapto-cytosine, isocytosine, pseudo-isocytosine, cytosine analogs with condensed ring systems (e.g., N,N′-propylene cytosine or phenoxazine), and uracil and its derivatives (e.g., 5-fluoro-uracil, 5-bromo-uracil, 5-bromovinyl-uracil, 4-thio-uracil, 5-hydroxy-uracil, 5-propynyl-uracil). Some of the preferred cytosines include 5-methyl-cytosine, 5-fluoro-cytosine, 5-hydroxy-cytosine, 5-hydroxymethyl-cytosine, and N4-ethyl-cytosine. In another embodiment of the invention, the cytosine base is substituted by a universal base (e.g., 3-nitropyrrole, P-base), an aromatic ring system (e.g., fluorobenzene or difluorobenzene) or a hydrogen atom (dSpacer).

A guanine may be replaced with a modified guanine base. A modified guanine as used herein is a naturally occurring or non-naturally occurring purine base analog of guanine which can replace this base without impairing the immunostimulatory activity of the oligonucleotide. Modified guanines include but are not limited to 7-deazaguanine, 7-deaza-7-substituted guanine (such as 7-deaza-7-(C2-C6)alkynylguanine), 7-deaza-8-substituted guanine, hypoxanthine, N2-substituted guanines (e.g., N2-methyl-guanine), 5-amino-3-methyl-3H,6H-thiazolo[4,5-d]pyrimidine-2,7-dione, 2,6-diaminopurine, 2-aminopurine, purine, indole, adenine, substituted adenines (e.g., N6-methyl-adenine, 8-oxo-adenine), 8-substituted guanine (e.g., 8-hydroxyguanine and 8-bromoguanine), and 6-thioguanine. In another embodiment of the invention, the guanine base is substituted by a universal base (e.g., 4-methyl-indole, 5-nitro-indole, and K-base), an aromatic ring system (e.g., benzimidazole or dichloro-benzimidazole, 1-methyl-1H-[1,2,4]triazole-3-carboxylic acid amide) or a hydrogen atom (dSpacer).

For use in the instant invention, the oligonucleotides of the invention can be synthesized de novo using any of a number of procedures well known in the art, for example, the β-cyanoethyl phosphoramidite method (Beaucage SL et al. (1981) Tetrahedron Lett 22:1859); or the nucleoside H-phosphonate method (Garegg et al. (1986) Tetrahedron Lett 27:4051-4; Froehler BC et al. (1986) Nucleic Acids Res 14:5399-407; Garegg et al. (1986) Tetrahedron Lett 27:4055-8; Gaffney et al. (1988) Tetrahedron Lett 29:2619-22). These chemistries can be performed by a variety of automated nucleic acid synthesizers available in the market. These oligonucleotides are referred to as synthetic oligonucleotides. An isolated oligonucleotide generally refers to an oligonucleotide which is separated from components which it is normally associated with in nature. As an example, an isolated oligonucleotide may be one which is separated from a cell, from a nucleus, from mitochondria or from chromatin.

Modified backbones such as phosphorothioates may be synthesized using automated techniques employing either phosphoramidate or H-phosphonate chemistries. Aryl-and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (e.g., Uhlmann E et al. (1990) Chem Rev 90:544; Goodchild J (1990) Bioconjugate Chem 1:165).

In each of the foregoing aspects of the invention, the composition can also further include a pharmaceutically acceptable carrier, such that the invention also provides pharmaceutical compositions containing the isolated inhibitory ODN of the invention.

The inhibitory ODN of the invention can also be used for the preparation of a medicament for use in treatment of a condition in a subject. The use according to this aspect of the invention involves the step of placing an effective amount of a composition of the invention in a pharmaceutically acceptable carrier.

Conjugates

In one aspect the invention provides a composition including a conjugate of an isolated immunoinhibitory nucleic acid molecule of the invention and an antigen or other therapeutic molecule. In one embodiment the antigen or other molecule is linked directly to the immunoinhibitory nucleic acid molecule of the invention, for example through a covalent bond. In one embodiment the antigen or other molecule is linked indirectly to the immunoinhibitory nucleic acid molecule of the invention, for example through a linker. When the antigen or other molecule of the conjugate is a polynucleotide encoding a peptide or polypeptide, the antigen or other molecule and the isolated immunoinhibitory nucleic acid molecule can be incorporated into a single expression vector. When the antigen or other molecule of the conjugate is a preformed polypeptide or polysaccharide, the antigen or other molecule and the isolated immunoinhibitory nucleic acid molecule can be linked using methods well known in the art.

The conjugate can include one or more isolated immunoinhibitory nucleic acid molecules of the invention. The conjugate can, alternatively or in addition, include one or more other molecules.

In one embodiment the conjugate includes an isolated immunoinhibitory nucleic acid molecule of the invention and a second molecule that is a TLR ligand or TLR agonist. More specifically, the TLR ligand or TLR agonist of the conjugate may be chosen from ligands and agonists of TLR7, TLR8, or TLR9. For example, in one embodiment an isolated immunoinhibitory nucleic acid molecule of the invention may be conjugated with an immunostimulatory CpG nucleic acid molecule. More particularly, in one embodiment an isolated TLR8 antagonist of the invention may be conjugated with an immunostimulatory CpG nucleic acid molecule. In one embodiment an isolated TLR7 antagonist of the invention may be conjugated with an immunostimulatory CpG nucleic acid molecule. In yet a further embodiment an isolated TLR7 antagonist of the invention and an isolated TLR8 antagonist of the invention may be conjugated with an immunostimulatory CpG nucleic acid molecule, wherein the TLR7 antagonist and the TLR8 antagonist may be present in a single molecular species or in separate molecular species.

As a further example of conjugates involving an isolated immunoinhibitory nucleic acid molecule of the invention and a second molecule that is a TLR ligand or TLR agonist, in one embodiment the conjugate includes a small molecule agonist of TLR7 or TLR8 (e.g., R-837 or R-848) and an isolated immunoinhibitory nucleic acid molecule of the invention (e.g., a TLR9 antagonist).

In some embodiments the conjugate includes two or more isolated immunoinhibitory nucleic acid molecules of the invention. The isolated immunoinhibitory nucleic acid molecules of the invention may be identical, may be different but selected from a single category (e.g., both TLR9 antagonists), or different and selected from different categories (e.g., a TLR9 antagonist and a TLR8 antagonist).

A conjugate that includes an isolated immunoinhibitory nucleic acid molecule of the invention and an antigen may be used to promote tolerance to the antigen. For example, it has been suggested that presentation of antigen to the immune system under circumstances which disfavor or prohibit development of a full immune response, e.g., by inhibiting costimulatory signals, can favor or induce so-called peripheral tolerance to the antigen. This type of tolerance is believed to reflect (clonal) anergy, in which antigen-specific T cells survive and are incapable of responding to the antigen upon subsequent presentation even in the context of adequate costimulation. Interestingly, it has been suggested that CpG ODN induce a signaling pathway in B cells that is most similar to CD40 costimulation, involving p38 and JNK, but not ERK-1 or ERK-2. Yi AK et al. (1998) J Immunol 161:4493-7; Lenert P et al. (2003) Antisense Nucleic Acid Drug Dev 13:143-50. It is also possible that the tolerance so induced may involve active immune suppression by so-called T-regulatory (Treg) cells. Treg cells may play an important role whenever the cytokine milieu is dominated by interleukin 10 (IL-10), i.e., the cytokine milieu is Th2-like; such a condition is believed to be favored by immunoinhibitory nucleic acid molecules of the invention. Whereas TLR agonists may block Treg suppressor activity (Pasare C et al. (2003) Science 299:1033-6), TLR antagonists may be permissive to Treg activity.

Use of Inhibitory ODN of the Invention

Further aspects of the invention relate to use of the inhibitory ODN of the invention. The oligonucleotides can be used alone or in combination with one another to inhibit signaling by TLR7, TLR8, or TLR9 individually or in any combination. Furthermore, the oligonucleotides can be used, alone or in combination with one another, in combination with an agonist or combination of agonists of any of TLR7, TLR8, or TLR9 to provide a combination inhibition/augmentation of signaling by any combination of TLR7, TLR8, and TLR9. The methods can be practiced in vitro and in vivo. Combinations of antagonists may involve separate molecules possessing the desired combination of antagonist motifs or they may involve single molecules possessing the desired combination of antagonist motifs. Similarly, combinations of agonists and antagonists may involve separate molecules possessing the desired combination of agonist and antagonist motifs or they may involve single molecules possessing the desired combination of agonist and antagonist motifs.

Inhibit TLR9 Alone

In one aspect the invention provides a method for inhibiting signaling by TLR9 with an inhibitory oligonucleotide. The method according to this aspect of the invention involves the step of contacting a cell or a population of cells expressing TLR9 with an effective amount of an inhibitory ODN that includes a TLR9-antagonist motif to inhibit signaling by TLR9. In one embodiment the method involves the step of contacting a cell or a population of cells expressing TLR9 with an effective amount of an inhibitory ODN that includes a TLR9-antagonist motif, wherein the TLR9-antagonist motif does not also include a TLR7-antagonist motif and wherein the TLR9-antagonist motif does not also include a TLR8-antagonist motif, to inhibit signaling by TLR9.

In one aspect the invention provides a method for inhibiting signaling by TLR9 in a subject with an inhibitory oligonucleotide. The method according to this aspect of the invention involves the step of administering to a subject an effective amount of an inhibitory ODN that includes a TLR9-antagonist motif to inhibit signaling by TLR9 in the subject. In one embodiment the method involves the step of administering to a subject an effective amount of an inhibitory ODN that includes a TLR9-antagonist motif, wherein the TLR9-antagonist motif does not also include a TLR7-antagonist motif and wherein the TLR9-antagonist motif does not also include a TLR8-antagonist motif, to inhibit signaling by TLR9 in the subject.

Inhibit TLR7, TLR8, and TLR9

In one aspect the invention provides a method for inhibiting signaling by TLR7, TLR8, and TLR9 with a single inhibitory oligonucleotide. The method according to this aspect of the invention involves the step of contacting a cell or a population of cells expressing at least one TLR chosen from TLR7, TLR8, TLR9, or any combination thereof, with an effective amount of an inhibitory ODN that includes a TLR7-antagonist motif, a TLR8-antagonist motif, and a TLR9-antagonist motif, to inhibit signaling by TLR7, TLR8, and TLR9. This method can be used whenever it is desirable to inhibit all three TLRs, although in certain embodiments only any one or only any two of the three TLRs may be expressed.

In one aspect the invention provides a method for inhibiting signaling by TLR7, TLR8, and TLR9 in a subject. The method according to this aspect of the invention involves the step of administering to a subject an effective amount of an inhibitory ODN that includes a TLR7-antagonist motif, a TLR8-antagonist motif, and a TLR9-antagonist motif, to inhibit signaling by TLR7, TLR8, and TLR9 in the subject.

In one aspect the invention provides a method for inhibiting signaling by TLR7, TLR8, and TLR9 with a combination of inhibitory oligonucleotides. The method according to this aspect of the invention involves the steps of contacting a cell or a population of cells expressing at least one TLR chosen from TLR7, TLR8, TLR9, or any combination thereof, with an effective amount of a combination of an inhibitory oligonucleotides chosen from (a)(i) an oligonucleotide that includes a TLR9-antagonist motif and (ii) an oligonucleotide or combination of oligonucleotides that includes both a TLR7-antagonist motif and a TLR8-antagonist motif; (b)(i) an oligonucleotide or combination of oligonucleotides that includes both a TLR9-antagonist motif and a TLR7-antagonist motif and (ii) an oligonucleotide that includes a TLR8-antagonist motif; or (c)(i) an oligonucleotide or combination of oligonucleotides that includes both a TLR9-antagonist motif and a TLR8-antagonist motif and (ii) an oligonucleotide that includes a TLR7-antagonist motif, to inhibit signaling by TLR7, TLR8, and TLR9.

In one aspect the invention provides a method for inhibiting signaling by TLR7, TLR8, and TLR9 in a subject with a combination of inhibitory oligonucleotides. The method according to this aspect of the invention involves the steps of administering to a subject an effective amount of a combination of an inhibitory oligonucleotides chosen from (a)(i) an oligonucleotide that includes a TLR9-antagonist motif and (ii) an oligonucleotide or combination of oligonucleotides that includes both a TLR7-antagonist motif and a TLR8-antagonist motif; (b)(i) an oligonucleotide or combination of oligonucleotides that includes both a TLR9-antagonist motif and a TLR7-antagonist motif and (ii) an oligonucleotide that includes a TLR8-antagonist motif; or (c)(i) an oligonucleotide or combination of oligonucleotides that includes both a TLR9-antagonist motif and a TLR8-antagonist motif and (ii) an oligonucleotide that includes a TLR7-antagonist motif, to inhibit signaling by TLR7, TLR8, and TLR9 in the subject.

Inhibit TLR8 and TLR9

In one aspect the invention provides a method for inhibiting signaling by TLR8 and TLR9 with a single inhibitory oligonucleotide. The method according to this aspect of the invention involves the step of contacting a cell or a population of cells expressing at least one TLR chosen from TLR8, TLR9, or a combination thereof, with an effective amount of an inhibitory ODN that includes a TLR8-antagonist motif and a TLR9-antagonist motif, to inhibit signaling by TLR8 and TLR9.

In one aspect the invention provides a method for inhibiting signaling by TLR8 and TLR9 in a subject. The method according to this aspect of the invention involves the step of administering to a subject an effective amount of an inhibitory ODN that includes a TLR8-antagonist motif and a TLR9-antagonist motif, to inhibit signaling by TLR8 and TLR9 in the subject.

In one aspect the invention provides a method for inhibiting signaling by TLR8 and TLR9 with a combination of inhibitory oligonucleotides. The method according to this aspect of the invention involves the steps of contacting a cell or a population of cells expressing at least one TLR chosen from TLR8, TLR9, or a combination thereof, with an effective amount of a combination of an oligonucleotide that includes a TLR9-antagonist motif and an oligonucleotide that includes a TLR8-antagonist motif, to inhibit signaling by TLR8 and TLR9.

In one aspect the invention provides a method for inhibiting signaling by TLR8 and TLR9 in a subject with a combination of inhibitory oligonucleotides. The method according to this aspect of the invention involves the steps of administering to a subject an effective amount of a combination of an oligonucleotide that includes a TLR9-antagonist motif and an oligonucleotide that includes a TLR8-antagonist motif, to inhibit signaling by TLR8 and TLR9 in the subject.

Inhibit TLR7 and TLR9

In one aspect the invention provides a method for inhibiting signaling by TLR7 and TLR9 with a single inhibitory oligonucleotide. The method according to this aspect of the invention involves the step of contacting a cell or a population of cells expressing at least one TLR chosen from TLR7, TLR9, or a combination thereof, with an effective amount of an inhibitory ODN that includes a TLR7-antagonist motif and a TLR9-antagonist motif, to inhibit signaling by TLR7 and TLR9.

In one aspect the invention provides a method for inhibiting signaling by TLR7 and TLR9 in a subject. The method according to this aspect of the invention involves the step of administering to a subject an effective amount of an inhibitory ODN that includes a TLR7-antagonist motif and a TLR9-antagonist motif, to inhibit signaling by TLR7 and TLR9 in the subject.

In one aspect the invention provides a method for inhibiting signaling by TLR7 and TLR9 with a combination of inhibitory oligonucleotides. The method according to this aspect of the invention involves the steps of contacting a cell or a population of cells expressing at least one TLR chosen from TLR7, TLR9, or a combination thereof, with an effective amount of a combination of an oligonucleotide that includes a TLR9-antagonist motif and an oligonucleotide that includes a TLR7-antagonist motif, to inhibit signaling by TLR7 and TLR9.

In one aspect the invention provides a method for inhibiting signaling by TLR7 and TLR9 in a subject with a combination of inhibitory oligonucleotides. The method according to this aspect of the invention involves the steps of administering to a subject an effective amount of a combination of an oligonucleotide that includes a TLR9-antagonist motif and an oligonucleotide that includes a TLR7-antagonist motif, to inhibit signaling by TLR7 and TLR9 in the subject.

Stimulate TLR7 and Inhibit TLR8 and TLR9

In one aspect the invention provides a method for inhibiting signaling by TLR8 and TLR9 and promoting signaling by TLR7 with a single oligonucleotide. The method according to this aspect of the invention involves the step of contacting a cell or a population of cells expressing at least one TLR chosen from TLR7, TLR8, TLR9, or any combination thereof, with an effective amount of an oligonucleotide that includes a TLR7-agonist motif, a TLR8-antagonist motif, and a TLR9-antagonist motif, to inhibit signaling by TLR8 and TLR9 and to promote signaling by TLR7.

In one aspect the invention provides a method for inhibiting signaling by TLR8 and TLR9 and promoting signaling by TLR7 in a subject. The method according to this aspect of the invention involves the step of administering to a subject an effective amount of an oligonucleotide that includes a TLR7-agonist motif, a TLR8-antagonist motif, and a TLR9-antagonist motif, to inhibit signaling by TLR8 and TLR9 and to promote signaling by TLR7 in the subject.

In one aspect the invention provides a method for inhibiting signaling by TLR8 and TLR9 and promoting signaling by TLR7 with a combination of agents including at least one inhibitory oligonucleotide of the invention. The method according to this aspect of the invention involves the steps of contacting a cell or a population of cells expressing at least one TLR chosen from TLR7, TLR8, TLR9, or any combination thereof, with an effective amount of a combination of agents chosen from (a)(i) an oligonucleotide that includes a TLR9-antagonist motif and (ii) an oligonucleotide or combination of oligonucleotides that includes both a TLR7-agonist motif and a TLR8-antagonist motif, (b)(i) an oligonucleotide that includes a TLR9-antagonist motif and (ii) a combination of a TLR7 agonist and an oligonucleotide that includes a TLR8-antagonist motif; (c)(i) an oligonucleotide or combination of oligonucleotides that includes both a TLR9-antagonist motif and a TLR7-agonist motif and (ii) an oligonucleotide that includes a TLR8-antagonist motif; (d)(i) a combination of a TLR7-agonist and an oligonucleotide that includes a TLR9-antagonist motif and (ii) an oligonucleotide that includes a TLR8-antagonist motif; (e)(i) an oligonucleotide or combination of oligonucleotides that includes both a TLR9-antagonist motif and a TLR8-antagonist motif and (ii) an oligonucleotide that includes a TLR7-agonist motif; or (f)(i) an oligonucleotide or combination of oligonucleotides that includes both a TLR9-antagonist motif and a TLR8-antagonist motif and (ii) a TLR7 agonist, to inhibit signaling by TLR8 and TLR9 and to promote signaling by TLR7.

In one aspect the invention provides a method for inhibiting signaling by TLR8 and TLR9 and promoting signaling by TLR7 in a subject with a combination of agents including at least one inhibitory oligonucleotide of the invention. The method according to this aspect of the invention involves the steps of administering to a subject an effective amount of a combination of agents chosen from (a)(i) an oligonucleotide that includes a TLR9-antagonist motif and (ii) an oligonucleotide or combination of oligonucleotides that includes both a TLR7-agonist motif and a TLR8-antagonist motif; (b)(i) an oligonucleotide that includes a TLR9-antagonist motif and (ii) a combination of a TLR7 agonist and an oligonucleotide that includes a TLR8-antagonist motif; (c)(i) an oligonucleotide or combination of oligonucleotides that includes both a TLR9-antagonist motif and a TLR7-agonist motif and (ii) an oligonucleotide that includes a TLR8-antagonist motif; (d)(i) a combination of a TLR7-agonist and an oligonucleotide that includes a TLR9-antagonist motif and (ii) an oligonucleotide that includes a TLR8-antagonist motif; (e)(i) an oligonucleotide or combination of oligonucleotides that includes both a TLR9-antagonist motif and a TLR8-antagonist motif and (ii) an oligonucleotide that includes a TLR7-agonist motif; or (f)(i) an oligonucleotide or combination of oligonucleotides that includes both a TLR9-antagonist motif and a TLR8-antagonist motif and (ii) a TLR7 agonist, to inhibit signaling by TLR8 and TLR9 and to promote signaling by TLR7 in the subject.

Stimulate TLR8 and Inhibit TLR7 and TLR9

In one aspect the invention provides a method for inhibiting signaling by TLR7 and TLR9 and promoting signaling by TLR8 with a single oligonucleotide. The method according to this aspect of the invention involves the step of contacting a cell or a population of cells expressing at least one TLR chosen from TLR7, TLR8, TLR9, or any combination thereof, with an effective amount of an oligonucleotide that includes a TLR8-agonist motif, a TLR7-antagonist motif, and a TLR9-antagonist motif, to inhibit signaling by TLR7 and TLR9 and to promote signaling by TLR8.

In one aspect the invention provides a method for inhibiting signaling by TLR7 and TLR9 and promoting signaling by TLR8 in a subject. The method according to this aspect of the invention involves the step of administering to a subject an effective amount of an oligonucleotide that includes a TLR8-agonist motif, a TLR7-antagonist motif, and a TLR9-antagonist motif, to inhibit signaling by TLR7 and TLR9 and to promote signaling by TLR8 in the subject.

In one aspect the invention provides a method for inhibiting signaling by TLR7 and TLR9 and promoting signaling by TLR8 with a combination of agents including at least one inhibitory oligonucleotide of the invention. The method according to this aspect of the invention involves the steps of contacting a cell or a population of cells expressing at least one TLR chosen from TLR7, TLR8, TLR9, or any combination thereof, with an effective amount of a combination of agents chosen from (a)(i) an oligonucleotide that includes a TLR9-antagonist motif and (ii) an oligonucleotide or combination of oligonucleotides that includes both a TLR8-agonist motif and a TLR7-antagonist motif; (b)(i) an oligonucleotide that includes a TLR9-antagonist motif and (ii) a combination of a TLR8 agonist and an oligonucleotide that includes a TLR7-antagonist motif; (c)(i) an oligonucleotide or combination of oligonucleotides that includes both a TLR9-antagonist motif and a TLR8-agonist motif and (ii) an oligonucleotide that includes a TLR7-antagonist motif; (d)(i) a combination of a TLR8-agonist and an oligonucleotide that includes a TLR9-antagonist motif and (ii) an oligonucleotide that includes a TLR7-antagonist motif; (e)(i) an oligonucleotide or combination of oligonucleotides that includes both a TLR9-antagonist motif and a TLR7-antagonist motif and (ii) an oligonucleotide that includes a TLR8-agonist motif; or (f)(i) an oligonucleotide or combination of oligonucleotides that includes both a TLR9-antagonist motif and a TLR7-antagonist motif and (ii) a TLR8 agonist, to inhibit signaling by TLR7 and TLR9 and to promote signaling by TLR8.

In one aspect the invention provides a method for inhibiting signaling by TLR7 and TLR9 and promoting signaling by TLR8 in a subject with a combination of agents including at least one inhibitory oligonucleotide of the invention. The method according to this aspect of the invention involves the steps of administering to a subject an effective amount of a combination of agents chosen from (a)(i) an oligonucleotide that includes a TLR9-antagonist motif and (ii) an oligonucleotide or combination of oligonucleotides that includes both a TLR8-agonist motif and a TLR7-antagonist motif; (b)(i) an oligonucleotide that includes a TLR9-antagonist motif and (ii) a combination of a TLR8 agonist and an oligonucleotide that includes a TLR7-antagonist motif; (c)(i) an oligonucleotide or combination of oligonucleotides that includes both a TLR9-antagonist motif and a TLR8-agonist motif and (ii) an oligonucleotide that includes a TLR7-antagonist motif; (d)(i) a combination of a TLR8-agonist and an oligonucleotide that includes a TLR9-antagonist motif and (ii) an oligonucleotide that includes a TLR7-antagonist motif; (e)(i) an oligonucleotide or combination of oligonucleotides that includes both a TLR9-antagonist motif and a TLR7-antagonist antagonist motif and (ii) an oligonucleotide that includes a TLR8-agonist motif; or (f)(i) an oligonucleotide or combination of oligonucleotides that includes both a TLR9-antagonist motif and a TLR7-antagonist motif and (ii) a TLR8 agonist, to inhibit signaling by TLR7 and TLR9 and to promote signaling by TLR8, to inhibit signaling by TLR7 and TLR9 and to promote signaling by TLR8 in the subject.

Inhibit TLR9 and Stimulate TLR7 and TLR8

In one aspect the invention provides a method for inhibiting signaling by TLR9 and promoting signaling by TLR7 and TLR8 with a single oligonucleotide. The method according to this aspect of the invention involves the step of contacting a cell or a population of cells expressing at least one TLR chosen from TLR7, TLR8, TLR9, or any combination thereof, with an effective amount of an oligonucleotide that includes a TLR7-agonist motif, a TLR8-agonist motif, and a TLR9-antagonist motif, to inhibit signaling by TLR9 and to promote signaling by TLR7 and TLR8.

In one aspect the invention provides a method for inhibiting signaling by TLR9 and promoting signaling by TLR7 and TLR8 in a subject. The method according to this aspect of the invention involves the step of administering to a subject an effective amount of an oligonucleotide that includes a TLR7-agonist motif, a TLR8-agonist motif, and a TLR9-antagonist motif, to inhibit signaling by TLR9 and to promote signaling by TLR7 and TLR8 in the subject.

In one aspect the invention provides a method for inhibiting signaling by TLR9 and promoting signaling by TLR7 and TLR8 with a combination of agents including at least one inhibitory oligonucleotide of the invention. The method according to this aspect of the invention involves the steps of contacting a cell or a population of cells expressing at least one TLR chosen from TLR7, TLR8, TLR9, or any combination thereof, with an effective amount of a combination of agents chosen from (a)(i) an oligonucleotide that includes a TLR9-antagonist motif and (ii) an oligonucleotide or combination of oligonucleotides that includes both a TLR8-agonist motif and a TLR7-agonist motif; (b)(i) an oligonucleotide that includes a TLR9-antagonist motif and (ii) a combination of a TLR8 agonist and an oligonucleotide that includes a TLR7-agonist motif; (c)(i) an oligonucleotide that includes a TLR9-antagonist motif and (ii) a combination of a TLR7 agonist and an oligonucleotide that includes a TLR8-agonist motif; (d)(i) an oligonucleotide that includes a TLR9-antagonist motif and (ii) a combination of a TLR8 agonist and a TLR7 agonist; (e)(i) an oligonucleotide or combination of oligonucleotides that includes both a TLR9-antagonist motif and a TLR8-agonist motif and (ii) an oligonucleotide that includes a TLR7-agonist motif; (f)(i) an oligonucleotide or combination of oligonucleotides that includes both a TLR9-antagonist motif and a TLR8-agonist motif and (ii) a TLR7 agonist; (g)(i) a combination of a TLR8 agonist and an oligonucleotide that includes a TLR9-antagonist motif and (ii) an oligonucleotide that includes a TLR7-agonist motif; (h)(i) a combination of a TLR8 agonist and an oligonucleotide that includes a TLR9-antagonist motif and (ii) a TLR7 agonist; (i)(i) an oligonucleotide or combination of oligonucleotides that includes both a TLR9-antagonist motif and a TLR7-agonist motif and (ii) an oligonucleotide that includes a TLR8-agonist motif; (j)(i) an oligonucleotide or combination of oligonucleotides that includes both a TLR9-antagonist motif and a TLR7-agonist motif and (ii) a TLR8 agonist; (k)(i) a combination of a TLR7 agonist and an oligonucleotide that includes a TLR9-antagonist motif and (ii) an oligonucleotide that includes a TLR8-agonist motif; or (l)(i) a combination of a TLR7 agonist and an oligonucleotide that includes a TLR9-antagonist motif and (ii) a TLR8 agonist, to inhibit signaling by TLR9 and to promote signaling by TLR7 and TLR8.

In one aspect the invention provides method for inhibiting signaling by TLR9 and promoting signaling by TLR7 and TLR8 in a subject with a combination of agents including at least one inhibitory oligonucleotide of the invention. The method according to this aspect of the invention involves the steps of administering to a subject an effective amount of a combination of agents chosen from (a)(i) an oligonucleotide that includes a TLR9-antagonist motif and (ii) an oligonucleotide or combination of oligonucleotides that includes both a TLR8-agonist motif and a TLR7-agonist motif; (b)(i) an oligonucleotide that includes a TLR9-antagonist motif and (ii) a combination of a TLR8 agonist and an oligonucleotide that includes a TLR7-agonist motif; (c)(i) an oligonucleotide that includes a TLR9-antagonist motif and (ii) a combination of a TLR7 agonist and an oligonucleotide that includes a TLR8-agonist motif; (d)(i) an oligonucleotide that includes a TLR9-antagonist motif and (ii) a combination of a TLR8 agonist and a TLR7 agonist; (e)(i) an oligonucleotide or combination of oligonucleotides that includes both a TLR9-antagonist motif and a TLR8-agonist motif and (ii) an oligonucleotide that includes a TLR7-agonist motif; (f)(i) an oligonucleotide or combination of oligonucleotides that includes both a TLR9-antagonist motif and a TLR8-agonist motif and (ii) a TLR7 agonist; (g)(i) a combination of a TLR8 agonist and an oligonucleotide that includes a TLR9-antagonist motif and (ii) an oligonucleotide that includes a TLR7-agonist motif; (h)(i) a combination of a TLR8 agonist and an oligonucleotide that includes a TLR9-antagonist motif and (ii) a TLR7 agonist; (i)(i) an oligonucleotide or combination of oligonucleotides that includes both a TLR9-antagonist motif and a TLR7-agonist motif and (ii) an oligonucleotide that includes a TLR8-agonist motif; (j)(i) an oligonucleotide or combination of oligonucleotides that includes both a TLR9-antagonist motif and a TLR7-agonist motif and (ii) a TLR8 agonist; (k)(i) a combination of a TLR7 agonist and an oligonucleotide that includes a TLR9-antagonist motif and (ii) an oligonucleotide that includes a TLR8-agonist motif; or (l)(i) a combination of a TLR7 agonist and an oligonucleotide that includes a TLR9-antagonist motif and (ii) a TLR8 agonist, to inhibit signaling by TLR9 and to promote signaling by TLR7 and TLR8 in the subject.

Inhibit TLR9 and Stimulate TLR7

In one aspect the invention provides a method for inhibiting signaling by TLR9 and promoting signaling by TLR7 with a single oligonucleotide. The method according to this aspect of the invention involves the step of contacting a cell or a population of cells expressing at least one TLR chosen from TLR7, TLR9, or a combination thereof, with an effective amount of an oligonucleotide that includes a TLR7-agonist motif and a TLR9-antagonist motif, to inhibit signaling by TLR9 and to promote signaling by TLR7.

In one aspect the invention provides a method for inhibiting signaling by TLR9 and promoting signaling by TLR7 in a subject. The method according to this aspect of the invention involves the step of administering to a subject an effective amount of an oligonucleotide that includes a TLR7-agonist motif and a TLR9-antagonist motif, to inhibit signaling by TLR9 and to promote signaling by TLR7 in the subject.

In one aspect the invention provides a method for inhibiting signaling by TLR9 and promoting signaling by TLR7 with a combination of agents including at least one inhibitory oligonucleotide of the invention. The method according to this aspect of the invention involves the step of contacting a cell or a population of cells expressing at least one TLR chosen from TLR7, TLR9, or a combination thereof, with an effective amount of a combination of agents chosen from (a)(i) an oligonucleotide that includes a TLR9-antagonist motif and (ii) an oligonucleotide that includes a TLR7-agonist motif; or (b)(i) an oligonucleotide that includes a TLR9-antagonist motif and (ii) a TLR7 agonist, to inhibit signaling by TLR9 and to promote signaling by TLR7.

In one aspect the invention provides a method for inhibiting signaling by TLR9 and promoting signaling by TLR7 in a subject with a combination of agents including at least one inhibitory oligonucleotide of the invention. The method according to this aspect of the invention involves the step of administering to a subject an effective amount of a combination of agents chosen from (a)(i) an oligonucleotide that includes a TLR9-antagonist motif and (ii) an oligonucleotide that includes a TLR7-agonist motif; or (b)(i) an oligonucleotide that includes a TLR9-antagonist motif and (ii) a TLR7 agonist, to inhibit signaling by TLR9 and to promote signaling by TLR7 in the subject.

Inhibit TLR9 and Stimulate TLR8

In one aspect the invention provides a method for inhibiting signaling by TLR9 and promoting signaling by TLR8 with a single oligonucleotide. The method according to this aspect of the invention involves the step of contacting a cell or a population of cells expressing at least one TLR chosen from TLR8, TLR9, or a combination thereof, with an effective amount of an oligonucleotide that includes a TLR8-agonist motif and a TLR9-antagonist motif, to inhibit signaling by TLR9 and to promote signaling by TLR8.

In one aspect the invention provides a method for inhibiting signaling by TLR9 and promoting signaling by TLR8 in a subject. The method according to this aspect of the invention involves the step of administering to a subject an effective amount of an oligonucleotide that includes a TLR8-agonist motif and a TLR9-antagonist motif, to inhibit signaling by TLR9 and to promote signaling by TLR8 in the subject.

In one aspect the invention provides a method for inhibiting signaling by TLR9 and promoting signaling by TLR8 with a combination of agents including at least one inhibitory oligonucleotide of the invention. The method according to this aspect of the invention involves the step of contacting a cell or a population of cells expressing at least one TLR chosen from TLR8, TLR9, or a combination thereof, with an effective amount of a combination of agents chosen from (a)(i) an oligonucleotide that includes a TLR9-antagonist motif and (ii) an oligonucleotide that includes a TLR8-agonist motif; or (b)(i) an oligonucleotide that includes a TLR9-antagonist motif and (ii) a TLR8 agonist, to inhibit signaling by TLR9 and to promote signaling by TLR8.

In one aspect the invention provides a method for inhibiting signaling by TLR9 and promoting signaling by TLR8 in a subject with a combination of agents including at least one inhibitory oligonucleotide of the invention. The method according to this aspect of the invention involves the step of administering to a subject an effective amount of a combination of agents chosen from (a)(i) an oligonucleotide that includes a TLR9-antagonist motif and (ii) an oligonucleotide that includes a TLR8-agonist motif; or (b)(i) an oligonucleotide that includes a TLR9-antagonist motif and (ii) a TLR8 agonist, to inhibit signaling by TLR9 and to promote signaling by TLR8 in the subject.

Stimulate TLR9 and Inhibit TLR7 and TLR8

In one aspect the invention provides a method for promoting signaling by TLR9 and inhibiting signaling by TLR7 and TLR8 with a single oligonucleotide. The method according to this aspect of the invention involves the step of contacting a cell or a population of cells expressing at least one TLR chosen from TLR7, TLR8, TLR9, or any combination thereof, with an effective amount of an oligonucleotide that includes a TLR7-antagonist motif, a TLR8-antagonist motif, and a TLR9-agonist motif, to promote signaling by TLR9 and to inhibit signaling by TLR7 and TLR8.

In one aspect the invention provides a method for promoting signaling by TLR9 and inhibiting signaling by TLR7 and TLR8 in a subject. The method according to this aspect of the invention involves the step of administering to a subject an effective amount of an oligonucleotide that includes a TLR7-antagonist motif, a TLR8-antagonist motif, and a TLR9-agonist motif, to promote signaling by TLR9 and to inhibit signaling by TLR7 and TLR8 in the subject.

In one aspect the invention provides a method for promoting signaling by TLR9 and inhibiting signaling by TLR7 and TLR8 with a combination of agents including at least one inhibitory oligonucleotide of the invention. The method according to this aspect of the invention involves the step of contacting a cell or a population of cells expressing at least one TLR chosen from TLR7, TLR8, TLR9, or any combination thereof, with an effective amount of a combination of agents chosen from (a)(i) an oligonucleotide that includes a TLR9-agonist motif and (ii) an oligonucleotide or combination of oligonucleotides that includes both a TLR8-antagonist motif and a TLR7-antagonist motif; (b)(i) a TLR9 agonist and (ii) an oligonucleotide or combination of oligonucleotides that includes both a TLR8-antagonist motif and a TLR7-antagonist motif; (c)(i) an oligonucleotide that includes a TLR9-agonist motif and (ii) a combination of a TLR8 antagonist and an oligonucleotide that includes a TLR7-antagonist motif; (d)(i) a TLR9 agonist and (ii) a combination of a TLR8 antagonist and an oligonucleotide that includes a TLR7-antagonist motif; (e)(i) an oligonucleotide that includes a TLR9-agonist motif and (ii) a combination of a TLR7 antagonist and an oligonucleotide that includes a TLR8-antagonist motif; (f)(i) a TLR9 agonist and (ii) a combination of a TLR7 antagonist and an oligonucleotide that includes a TLR8-antagonist motif; (g)(i) an oligonucleotide or combination of oligonucleotides that includes both a TLR9-agonist motif and a TLR8-antagonist motif and (ii) an oligonucleotide that includes a TLR7-antagonist motif; (h)(i) a combination of a TLR9 agonist and an oligonucleotide that includes a TLR8-antagonist motif and (ii) an oligonucleotide that includes a TLR7-antagonist motif; (i)(i) an oligonucleotide or combination of oligonucleotides that includes both a TLR9-agonist motif and a TLR8-antagonist motif and (ii) a TLR7 antagonist; (j) a combination of a TLR9 agonist and an oligonucleotide that includes a TLR8-antagonist motif and (ii) a TLR7 antagonist; (k)(i) a combination of a TLR8 antagonist and an oligonucleotide that includes a TLR9-agonist motif and (ii) an oligonucleotide that includes a TLR7-antagonist motif; (l)(i) a combination of a TLR8 antagonist and a TLR9 agonist and (ii) an oligonucleotide that includes a TLR7-antagonist motif; (m)(i) an oligonucleotide or combination of oligonucleotides that includes both a TLR9-agonist motif and a TLR7-antagonist motif and (ii) an oligonucleotide that includes a TLR8-antagonist motif; (n)(i) a combination of a TLR9 agonist and an oligonucleotide that includes a TLR7-antagonist motif and (ii) an oligonucleotide that includes a TLR8-antagonist motif; (o)(i) an oligonucleotide or combination of oligonucleotides that includes both a TLR9-agonist motif and a TLR7-antagonist motif and (ii) a TLR8 antagonist; (p)(i) a combination of a TLR9 agonist and an oligonucleotide that includes a TLR7-antagonist motif and (ii) a TLR8 antagonist; (q)(i) a combination of a TLR7 antagonist and an oligonucleotide that includes a TLR9-agonist motif and (ii) an oligonucleotide that includes a TLR8-antagonist motif; or (r)(i) a combination of a TLR7 antagonist a TLR9 agonist and (ii) an oligonucleotide that includes a TLR8-antagonist motif, to promote signaling by TLR9 and to inhibit signaling by TLR7 and TLR8.

In one aspect the invention provides method for promoting signaling by TLR9 and inhibiting signaling by TLR7 and TLR8 in a subject with a combination of agents including at least one inhibitory oligonucleotide of the invention. The method according to this aspect of the invention involves the step of administering to a subject an effective amount of a combination of agents chosen from (a)(i) an oligonucleotide that includes a TLR9-agonist motif and (ii) an oligonucleotide or combination of oligonucleotides that includes both a TLR8-antagonist motif and a TLR7-antagonist motif; (b)(i) a TLR9 agonist and (ii) an oligonucleotide or combination of oligonucleotides that includes both a TLR8-antagonist motif and a TLR7-antagonist motif; (c)(i) an oligonucleotide that includes a TLR9-agonist motif and (ii) a combination of a TLR8 antagonist and an oligonucleotide that includes a TLR7-antagonist motif; (d)(i) a TLR9 agonist and (ii) a combination of a TLR8 antagonist and an oligonucleotide that includes a TLR7-antagonist motif; (e)(i) an oligonucleotide that includes a TLR9-agonist motif and (ii) a combination of a TLR7 antagonist and an oligonucleotide that includes a TLR8-antagonist motif; (f)(i) a TLR9 agonist and (ii) a combination of a TLR7 antagonist and an oligonucleotide that includes a TLR8-antagonist motif; (g)(i) an oligonucleotide or combination of oligonucleotides that includes both a TLR9-agonist motif and a TLR8-antagonist motif and (ii) an oligonucleotide that includes a TLR7-antagonist motif; (h)(i) a combination of a TLR9 agonist and an oligonucleotide that includes a TLR8-antagonist motif and (ii) an oligonucleotide that includes a TLR7-antagonist motif; (i)(i) an oligonucleotide or combination of oligonucleotides that includes both a TLR9-agonist motif and a TLR8-antagonist motif and (ii) a TLR7 antagonist; (j) a combination of a TLR9 agonist and an oligonucleotide that includes a TLR8-antagonist motif and (ii) a TLR7 antagonist; (k)(i) a combination of a TLR8 antagonist and an oligonucleotide that includes a TLR9-agonist motif and (ii) an oligonucleotide that includes a TLR7-antagonist motif; (l)(i) a combination of a TLR8 antagonist and a TLR9 agonist and (ii) an oligonucleotide that includes a TLR7-antagonist motif; (m)(i) an oligonucleotide or combination of oligonucleotides that includes both a TLR9-agonist motif and a TLR7-antagonist motif and (ii) an oligonucleotide that includes a TLR8-antagonist motif; (n)(i) a combination of a TLR9 agonist and an oligonucleotide that includes a TLR7-antagonist motif and (ii) an oligonucleotide that includes a TLR8-antagonist motif; (o)(i) an oligonucleotide or combination of oligonucleotides that includes both a TLR9-agonist motif and a TLR7-antagonist motif and (ii) a TLR8 antagonist; (p)(i) a combination of a TLR9 agonist and an oligonucleotide that includes a TLR7-antagonist motif and (ii) a TLR8 antagonist; (q)(i) a combination of a TLR7 antagonist and an oligonucleotide that includes a TLR9-agonist motif and (ii) an oligonucleotide that includes a TLR8-antagonist motif; or (r)(i) a combination of a TLR7 antagonist a TLR9 agonist and (ii) an oligonucleotide that includes a TLR8-antagonist motif, to promote signaling by TLR9 and to inhibit signaling by TLR7 and TLR8 in the subject.

Stimulate TLR9 and Inhibit TLR8

In one aspect the invention provides a method for inhibiting signaling by TLR8 and promoting signaling by TLR9 with a single oligonucleotide. The method according to this aspect of the invention involves the step of contacting a cell or a population of cells expressing at least one TLR chosen from TLR8, TLR9, or a combination thereof, with an effective amount of an oligonucleotide that includes a TLR9-agonist motif and a TLR8-antagonist motif, to inhibit signaling by TLR8 and to promote signaling by TLR9.

In one aspect the invention provides a method for inhibiting signaling by TLR8 and promoting signaling by TLR9 in a subject. The method according to this aspect of the invention involves the step of administering to a subject an effective amount of an oligonucleotide that includes a TLR9-agonist motif and a TLR8-antagonist motif, to inhibit signaling by TLR8 and to promote signaling by TLR9 in the subject.

In one aspect the invention provides a method for inhibiting signaling by TLR8 and promoting signaling by TLR9 with a combination of agents including at least one inhibitory oligonucleotide of the invention. The method according to this aspect of the invention involves the step of contacting a cell or a population of cells expressing at least one TLR chosen from TLR8, TLR9, or a combination thereof, with an effective amount of a combination of agents chosen from (a)(i) an oligonucleotide that includes a TLR8-antagonist motif and (ii) an oligonucleotide that includes a TLR9-agonist motif; or (b)(i) an oligonucleotide that includes a TLR8-antagonist motif and (ii) a TLR9 agonist, to inhibit signaling by TLR8 and to promote signaling by TLR9.

In one aspect the invention provides a method for inhibiting signaling by TLR8 and promoting signaling by TLR9 in a subject with a combination of agents including at least one inhibitory oligonucleotide of the invention. The method according to this aspect of the invention involves the step of administering to a subject an effective amount of a combination of agents chosen from (a)(i) an oligonucleotide that includes a TLR8-antagonist motif and (ii) an oligonucleotide that includes a TLR9-agonist motif; or (b)(i) an oligonucleotide that includes a TLR8-antagonist motif and (ii) a TLR9 agonist, to inhibit signaling by TLR8 and to promote signaling by TLR9 in the subject.

Stimulate TLR9 and Inhibit TLR7

In one aspect the invention provides a method for inhibiting signaling by TLR7 and promoting signaling by TLR9 with a single oligonucleotide. The method according to this aspect of the invention involves the step of contacting a cell or a population of cells expressing at least one TLR chosen from TLR7, TLR9, or a combination thereof, with an effective amount of an oligonucleotide that includes a TLR9-agonist motif and a TLR7-antagonist motif, to inhibit signaling by TLR7 and to promote signaling by TLR9.

In one aspect the invention provides a method for inhibiting signaling by TLR7 and promoting signaling by TLR9 in a subject. The method according to this aspect of the invention involves the step of administering to a subject an effective amount of an oligonucleotide that includes a TLR9-agonist motif and a TLR7-antagonist motif, to inhibit signaling by TLR7 and to promote signaling by TLR9 in the subject.

In one aspect the invention provides a method for inhibiting signaling by TLR7 and promoting signaling by TLR9 with a combination of agents including at least one inhibitory oligonucleotide of the invention. The method according to this aspect of the invention involves the step of contacting a cell or a population of cells expressing at least one TLR chosen from TLR7, TLR9, or a combination thereof, with an effective amount of a combination of agents chosen from (a)(i) an oligonucleotide that includes a TLR7-antagonist motif and (ii) an oligonucleotide that includes a TLR9-agonist motif; or (b)(i) an oligonucleotide that includes a TLR7-antagonist motif and (ii) a TLR9 agonist, to inhibit signaling by TLR7 and to promote signaling by TLR9.

In one aspect the invention provides a method for inhibiting signaling by TLR7 and promoting signaling by TLR9 in a subject with a combination of agents including at least one inhibitory oligonucleotide of the invention. The method according to this aspect of the invention involves the step of administering to a subject an effective amount of a combination of agents chosen from (a)(i) an oligonucleotide that includes a TLR7-antagonist motif and (ii) an oligonucleotide that includes a TLR9-agonist motif; or (b)(i) an oligonucleotide that includes a TLR7-antagonist motif and (ii) a TLR9 agonist, to inhibit signaling by TLR7 and to promote signaling by TLR9 in the subject.

Stimulate TLR8 and TLR9 and Inhibit TLR7

In one aspect the invention provides a method for promoting signaling by TLR8 and TLR9 and inhibiting signaling by TLR7 with a single oligonucleotide. The method according to this aspect of the invention involves the step of contacting a cell or a population of cells expressing at least one TLR chosen from TLR7, TLR8, TLR9, or any combination thereof, with an effective amount of an oligonucleotide that includes a TLR7-antagonist motif, a TLR8-agonist motif, and a TLR9-agonist motif, to promote signaling by TLR8 and TLR9 and to inhibit signaling by TLR7.

In one aspect the invention provides a method for promoting signaling by TLR8 and TLR9 and inhibiting signaling by TLR7 in a subject. The method according to this aspect of the invention involves the step of administering to a subject an effective amount of an oligonucleotide that includes a TLR7-antagonist motif, a TLR8-agonist motif, and a TLR9-agonist motif, to promote signaling by TLR8 and TLR9 and to inhibit signaling by TLR7 in the subject.

In one aspect the invention provides a method for promoting signaling by TLR8 and TLR9 and inhibiting signaling by TLR7 with a combination of agents including at least one inhibitory oligonucleotide of the invention. The method according to this aspect of the invention involves the step of contacting a cell or a population of cells expressing at least one TLR chosen from TLR7, TLR8, TLR9, or any combination thereof, with an effective amount of a combination of agents chosen from (a)(i) an oligonucleotide that includes a TLR9-agonist motif and (ii) an oligonucleotide or combination of oligonucleotides that includes both a TLR8-agonist motif and a TLR7-antagonist motif; (b)(i) a TLR9 agonist and (ii) an oligonucleotide or combination of oligonucleotides that includes both a TLR8-agonist motif and a TLR7-antagonist motif; (c)(i) an oligonucleotide that includes a TLR9-agonist motif and (ii) a combination of a TLR8 agonist and an oligonucleotide that includes a TLR7-antagonist motif; (d)(i) a TLR9 agonist and (ii) a combination of a TLR8 agonist and an oligonucleotide that includes a TLR7-antagonist motif; (e)(i) an oligonucleotide or combination of oligonucleotides that includes both a TLR9-agonist motif and a TLR8-agonist motif and (ii) an oligonucleotide that includes a TLR7-antagonist motif; (f)(i) a combination of a TLR9 agonist and an oligonucleotide that includes a TLR8-agonist motif and (ii) an oligonucleotide that includes a TLR7-antagonist motif; (g)(i) a combination of a TLR8 agonist and an oligonucleotide that includes a TLR9-agonist motif and (ii) an oligonucleotide that includes a TLR7-antagonist motif; (h)(i) a combination of a TLR8 agonist and a TLR9 agonist and (ii) an oligonucleotide that includes a TLR7-antagonist motif; (i)(i) an oligonucleotide or combination of oligonucleotides that includes both a TLR9-agonist motif and a TLR7-antagonist motif and (ii) an oligonucleotide that includes a TLR8-agonist motif; (j)(i) a combination of a TLR9 agonist and an oligonucleotide that includes a TLR7-antagonist motif and (ii) an oligonucleotide that includes a TLR8-agonist motif; (k)(i) an oligonucleotide or combination of oligonucleotides that includes both a TLR9-agonist motif and a TLR7-antagonist motif and (ii) a TLR8 agonist; or (l)(i) a combination of a TLR9 agonist and an oligonucleotide that includes a TLR7-antagonist motif and (ii) a TLR8 agonist, to promote signaling by TLR8 and TLR9 and to inhibit signaling by TLR7.

In one aspect the invention provides method for promoting signaling by TLR8 and TLR9 and inhibiting signaling by TLR7 in a subject with a combination of agents including at least one inhibitory oligonucleotide of the invention. The method according to this aspect of the invention involves the step of administering to a subject an effective amount of a combination of agents chosen from (a)(i) an oligonucleotide that includes a TLR9-agonist motif and (ii) an oligonucleotide or combination of oligonucleotides that includes both a TLR8-agonist motif and a TLR7-antagonist motif; (b)(i) a TLR9 agonist and (ii) an oligonucleotide or combination of oligonucleotides that includes both a TLR8-agonist motif and a TLR7-antagonist motif; (c)(i) an oligonucleotide that includes a TLR9-agonist motif and (ii) a combination of a TLR8 agonist and an oligonucleotide that includes a TLR7-antagonist motif; (d)(i) a TLR9 agonist and (ii) a combination of a TLR8 agonist and an oligonucleotide that includes a TLR7-antagonist motif; (e)(i) an oligonucleotide or combination of oligonucleotides that includes both a TLR9-agonist motif and a TLR8-agonist motif and (ii) an oligonucleotide that includes a TLR7-antagonist motif; (f)(i) a combination of a TLR9 agonist and an oligonucleotide that includes a TLR8-agonist motif and (ii) an oligonucleotide that includes a TLR7-antagonist motif; (g)(i) a combination of a TLR8 agonist and an oligonucleotide that includes a TLR9-agonist motif and (ii) an oligonucleotide that includes a TLR7-antagonist motif; (h)(i) a combination of a TLR8 agonist and a TLR9 agonist and (ii) an oligonucleotide that includes a TLR7-antagonist motif; (i)(i) an oligonucleotide or combination of oligonucleotides that includes both a TLR9-agonist motif and a TLR7-antagonist motif and (ii) an oligonucleotide that includes a TLR8-agonist motif; (j)(i) a combination of a TLR9 agonist and an oligonucleotide that includes a TLR7-antagonist motif and (ii) an oligonucleotide that includes a TLR8-agonist motif; (k)(i) an oligonucleotide or combination of oligonucleotides that includes both a TLR9-agonist motif and a TLR7-antagonist motif and (ii) a TLR8 agonist; or (l)(i) a combination of a TLR9 agonist and an oligonucleotide that includes a TLR7-antagonist motif and (ii) a TLR8 agonist, to promote signaling by TLR8 and TLR9 and to inhibit signaling by TLR7 in the subject.

Stimulate TLR7 and TLR9 and Inhibit TLR8

In one aspect the invention provides a method for promoting signaling by TLR7 and TLR9 and inhibiting signaling by TLR8 with a single oligonucleotide. The method according to this aspect of the invention involves the step of contacting a cell or a population of cells expressing at least one TLR chosen from TLR7, TLR8, TLR9, or any combination thereof, with an effective amount of an oligonucleotide that includes a TLR8-antagonist motif, a TLR7-agonist motif, and a TLR9-agonist motif, to promote signaling by TLR7 and TLR9 and to inhibit signaling by TLR8.

In one aspect the invention provides a method for promoting signaling by TLR7 and TLR9 and inhibiting signaling by TLR8 in a subject. The method according to this aspect of the invention involves the step of administering to a subject an effective amount of an oligonucleotide that includes a TLR8-antagonist motif, a TLR7-agonist motif, and a TLR9-agonist motif, to promote signaling by TLR7 and TLR9 and to inhibit signaling by TLR8 in the subject.

In one aspect the invention provides a method for promoting signaling by TLR7 and TLR9 and inhibiting signaling by TLR8 with a combination of agents including at least one inhibitory oligonucleotide of the invention. The method according to this aspect of the invention involves the step of contacting a cell or a population of cells expressing at least one TLR chosen from TLR7, TLR8, TLR9, or any combination thereof, with an effective amount of a combination of agents chosen from (a)(i) an oligonucleotide that includes a TLR9-agonist motif and (ii) an oligonucleotide or combination of oligonucleotides that includes both a TLR7-agonist motif and a TLR8-antagonist motif; (b)(i) a TLR9 agonist and (ii) an oligonucleotide or combination of oligonucleotides that includes both a TLR7-agonist motif and a TLR8-antagonist motif; (c)(i) an oligonucleotide that includes a TLR9-agonist motif and (ii) a combination of a TLR7 agonist and an oligonucleotide that includes a TLR8-antagonist motif; (d)(i) a TLR9 agonist and (ii) a combination of a TLR7 agonist and an oligonucleotide that includes a TLR8-antagonist motif; (e)(i) an oligonucleotide or combination of oligonucleotides that includes both a TLR9-agonist motif and a TLR7-agonist motif and (ii) an oligonucleotide that includes a TLR8-antagonist motif; (f)(i) a combination of a TLR9 agonist and an oligonucleotide that includes a TLR7-agonist motif and (ii) an oligonucleotide that includes a TLR8-antagonist motif; (g)(i) a combination of a TLR7 agonist and an oligonucleotide that includes a TLR9-agonist motif and (ii) an oligonucleotide that includes a TLR8-antagonist motif; (h)(i) a combination of a TLR7 agonist and a TLR9 agonist and (ii) an oligonucleotide that includes a TLR8-antagonist motif; (i)(i) an oligonucleotide or combination of oligonucleotides that includes both a TLR9-agonist motif and a TLR8-antagonist motif and (ii) an oligonucleotide that includes a TLR7-agonist motif; (j)(i) a combination of a TLR9 agonist and an oligonucleotide that includes a TLR8-antagonist motif and (ii) an oligonucleotide that includes a TLR7-agonist motif; (k)(i) an oligonucleotide or combination of oligonucleotides that includes both a TLR9-agonist motif and a TLR8-antagonist motif and (ii) a TLR7 agonist; or (l)(i) a combination of a TLR9 agonist and an oligonucleotide that includes a TLR8-antagonist motif and (ii) a TLR7 agonist, to promote signaling by TLR7 and TLR9 and to inhibit signaling by TLR8.

In one aspect the invention provides method for promoting signaling by TLR7 and TLR9 and inhibiting signaling by TLR8 in a subject with a combination of agents including at least one inhibitory oligonucleotide of the invention. The method according to this aspect of the invention involves the step of administering to a subject an effective amount of a combination of agents chosen from (a)(i) an oligonucleotide that includes a TLR9-agonist motif and (ii) an oligonucleotide or combination of oligonucleotides that includes both a TLR7-agonist motif and a TLR8-antagonist motif; (b)(i) a TLR9 agonist and (ii) an oligonucleotide or combination of oligonucleotides that includes both a TLR7-agonist motif and a TLR8-antagonist motif; (c)(i) an oligonucleotide that includes a TLR9-agonist motif and (ii) a combination of a TLR7 agonist and an oligonucleotide that includes a TLR8-antagonist motif; (d)(i) a TLR9 agonist and (ii) a combination of a TLR7 agonist and an oligonucleotide that includes a TLR8-antagonist motif; (e)(i) an oligonucleotide or combination of oligonucleotides that includes both a TLR9-agonist motif and a TLR7-agonist motif and (ii) an oligonucleotide that includes a TLR8-antagonist motif; (f)(i) a combination of a TLR9 agonist and an oligonucleotide that includes a TLR7-agonist motif and (ii) an oligonucleotide that includes a TLR8-antagonist motif; (g)(i) a combination of a TLR7 agonist and an oligonucleotide that includes a TLR9-agonist motif and (ii) an oligonucleotide that includes a TLR8-antagonist motif; (h)(i) a combination of a TLR7 agonist and a TLR9 agonist and (ii) an oligonucleotide that includes a TLR8-antagonist motif; (i)(i) an oligonucleotide or combination of oligonucleotides that includes both a TLR9-agonist motif and a TLR8-antagonist motif and (ii) an oligonucleotide that includes a TLR7-agonist motif; (j)(i) a combination of a TLR9 agonist and an oligonucleotide that includes a TLR8-antagonist motif and (ii) an oligonucleotide that includes a TLR7-agonist motif; (k)(i) an oligonucleotide or combination of oligonucleotides that includes both a TLR9-agonist motif and a TLR8-antagonist motif and (ii) a TLR7 agonist; or (l)(i) a combination of a TLR9 agonist and an oligonucleotide that includes a TLR8-antagonist motif and (ii) a TLR7 agonist, to promote signaling by TLR7 and TLR9 and to inhibit signaling by TLR8 in the subject.

Inhibit TLR7 Alone

In one aspect the invention provides a method for inhibiting signaling by TLR7 with an inhibitory oligonucleotide. The method according to this aspect of the invention involves the step of contacting a cell or a population of cells expressing TLR7 with an effective amount of an inhibitory ODN that includes a TLR7-antagonist motif to inhibit signaling by TLR7. In one embodiment the method involves the step of contacting a cell or a population of cells expressing TLR7 with an effective amount of an inhibitory ODN that includes a TLR7-antagonist motif, wherein the TLR7-antagonist motif does not also include a TLR8-antagonist motif and wherein the TLR7-antagonist motif does not also include a TLR9-antagonist motif, to inhibit signaling by TLR7.

In one aspect the invention provides a method for inhibiting signaling by TLR7 in a subject with an inhibitory oligonucleotide. The method according to this aspect of the invention involves the step of administering to a subject an effective amount of an inhibitory ODN that includes a TLR7-antagonist motif to inhibit signaling by TLR7 in the subject. In one embodiment the method involves the step of administering to a subject an effective amount of an inhibitory ODN that includes a TLR7-antagonist motif, wherein the TLR7-antagonist motif does not also include a TLR8-antagonist motif and wherein the TLR7-antagonist motif does not also include a TLR9-antagonist motif, to inhibit signaling by TLR7 in the subject.

Inhibit TLR8 Alone

In one aspect the invention provides a method for inhibiting signaling by TLR8 with an inhibitory oligonucleotide. The method according to this aspect of the invention involves the step of contacting a cell or a population of cells expressing TLR8 with an effective amount of an inhibitory ODN that includes a TLR8-antagonist motif to inhibit signaling by TLR8. In one embodiment the method involves the step of contacting a cell or a population of cells expressing TLR8 with an effective amount of an inhibitory ODN that includes a TLR8-antagonist motif, wherein the TLR8-antagonist motif does not also include a TLR7-antagonist motif and wherein the TLR8-antagonist motif does not also include a TLR9-antagonist motif, to inhibit signaling by TLR8.

In one aspect the invention provides a method for inhibiting signaling by TLR8 in a subject with an inhibitory oligonucleotide. The method according to this aspect of the invention involves the step of administering to a subject an effective amount of an inhibitory ODN that includes a TLR8-antagonist motif to inhibit signaling by TLR8 in the subject. In one embodiment the method involves the step of administering to a subject an effective amount of an inhibitory ODN that includes a TLR8-antagonist motif, wherein the TLR8-antagonist motif does not also include a TLR7-antagonist motif and wherein the TLR8-antagonist motif does not also include a TLR9-antagonist motif, to inhibit signaling by TLR8 in the subject.

Inhibit TLR7 and TLR8

In one aspect the invention provides a method for inhibiting signaling by TLR7 and TLR8 with a single inhibitory oligonucleotide. The method according to this aspect of the invention involves the step of contacting a cell or a population of cells expressing at least one TLR chosen from TLR7, TLR8, or a combination thereof, with an effective amount of an inhibitory ODN that includes a TLR7-antagonist motif and a TLR8-antagonist motif, to inhibit signaling by TLR7 and TLR8.

In one aspect the invention provides a method for inhibiting signaling by TLR7 and TLR8 in a subject. The method according to this aspect of the invention involves the step of administering to a subject an effective amount of an inhibitory ODN that includes a TLR7-antagonist motif and a TLR8-antagonist motif, to inhibit signaling by TLR7 and TLR8 in the subject.

In one aspect the invention provides a method for inhibiting signaling by TLR7 and TLR8 with a combination of inhibitory oligonucleotides. The method according to this aspect of the invention involves the step of contacting a cell or a population of cells expressing at least one TLR chosen from TLR7, TLR8, or a combination thereof, with an effective amount of a combination of an oligonucleotide that includes a TLR8-antagonist motif and an oligonucleotide that includes a TLR7-antagonist motif, to inhibit signaling by TLR7 and TLR8.

In one aspect the invention provides a method for inhibiting signaling by TLR7 and TLR8 in a subject with a combination of inhibitory oligonucleotides. The method according to this aspect of the invention involves the step of administering to a subject an effective amount of a combination of an oligonucleotide that includes a TLR8-antagonist motif and an oligonucleotide that includes a TLR7-antagonist motif, to inhibit signaling by TLR7 and TLR8 in the subject.

Inhibit TLR7 and Stimulate TLR8

In one aspect the invention provides a method for promoting signaling by TLR8 and inhibiting signaling by TLR7 with a single oligonucleotide. The method according to this aspect of the invention involves the step of contacting a cell or a population of cells expressing at least one TLR chosen from TLR7, TLR8, or a combination thereof, with an effective amount of an oligonucleotide that includes a TLR7-antagonist motif and a TLR8-agonist motif, to promote signaling by TLR8 and to inhibit signaling by TLR7.

In one aspect the invention provides a method for promoting signaling by TLR8 and inhibiting signaling by TLR7 in a subject. The method according to this aspect of the invention involves the step of administering to a subject an effective amount of an oligonucleotide that includes a TLR7-antagonist motif and a TLR8-agonist motif, to promote signaling by TLR8 and to inhibit signaling by TLR7 in the subject.

In one aspect the invention provides a method for promoting signaling by TLR8 and inhibiting signaling by TLR7 with a combination of agents including at least one inhibitory oligonucleotide of the invention. The method according to this aspect of the invention involves the step of contacting a cell or a population of cells expressing at least one TLR chosen from TLR7, TLR8, or a combination thereof, with an effective amount of a combination of agents chosen from (a)(i) an oligonucleotide that includes a TLR8-agonist motif and (ii) an oligonucleotide that includes a TLR7-antagonist motif; or (b)(i) a TLR8 agonist and (ii) an oligonucleotide that includes a TLR7-antagonist motif, to promote signaling by TLR8 and to inhibit signaling by TLR7.

In one aspect the invention provides method for promoting signaling by TLR8 and inhibiting signaling by TLR7 in a subject with a combination of agents including at least one inhibitory oligonucleotide of the invention. The method according to this aspect of the invention involves the step of administering to a subject an effective amount of a combination of agents chosen from (a)(i) an oligonucleotide that includes a TLR8-agonist motif and (ii) an oligonucleotide that includes a TLR7-antagonist motif; or (b)(i) a TLR8 agonist and (ii) an oligonucleotide that includes a TLR7-antagonist motif, to promote signaling by TLR8 and to inhibit signaling by TLR7 in the subject.

Inhibit TLR8 and Stimulate TLR7

In one aspect the invention provides a method for promoting signaling by TLR7 and inhibiting signaling by TLR8 with a single oligonucleotide. The method according to this aspect of the invention involves the step of contacting a cell or a population of cells expressing at least one TLR chosen from TLR7, TLR8, or a combination thereof, with an effective amount of an oligonucleotide that includes a TLR8-antagonist motif and a TLR7-agonist motif, to promote signaling by TLR7 and to inhibit signaling by TLR8.

In one aspect the invention provides a method for promoting signaling by TLR7 and inhibiting signaling by TLR8 in a subject. The method according to this aspect of the invention involves the step of administering to a subject an effective amount of an oligonucleotide that includes a TLR8-antagonist motif and a TLR7-agonist motif, to promote signaling by TLR7 and to inhibit signaling by TLR8 in the subject.

In one aspect the invention provides a method for promoting signaling by TLR7 and inhibiting signaling by TLR8 with a combination of agents including at least one inhibitory oligonucleotide of the invention. The method according to this aspect of the invention involves the step of contacting a cell or a population of cells expressing at least one TLR chosen from TLR7, TLR8, or a combination thereof, with an effective amount of a combination of agents chosen from (a)(i) an oligonucleotide that includes a TLR7-agonist motif and (ii) an oligonucleotide that includes a TLR8-antagonist motif; or (b)(i) a TLR7 agonist and (ii) an oligonucleotide that includes a TLR8-antagonist motif, to promote signaling by TLR7 and to inhibit signaling by TLR8.

In one aspect the invention provides method for promoting signaling by TLR7 and inhibiting signaling by TLR8 in a subject with a combination of agents including at least one inhibitory oligonucleotide of the invention. The method according to this aspect of the invention involves the step of administering to a subject an effective amount of a combination of agents chosen from (a)(i) an oligonucleotide that includes a TLR7-agonist motif and (ii) an oligonucleotide that includes a TLR8-antagonist motif; or (b)(i) a TLR7 agonist and (ii) an oligonucleotide that includes a TLR8-antagonist motif, to promote signaling by TLR7 and to inhibit signaling by TLR8 in the subject.

The invention in another aspect provides a method for reducing an immunostimulatory effect of a CpG nucleic acid molecule. The method involves the step of contacting an immune cell that is sensitive to a CpG nucleic acid molecule with an effective amount of an isolated immunoinhibitory nucleic acid molecule of the invention to reduce an immunostimulatory effect of the CpG nucleic acid molecule on the immune cell to a level below that which would occur without the contacting.

In one embodiment the immunostimulatory effect that is inhibited is Th1-like skewing of an immune response. One feature of at least certain types of immunostimulatory CpG nucleic acids is their ability to skew an immune response toward a Th1-like profile and away from a Th2-like profile. This feature is believed to serve as a basis for the observed efficacy of immunostimulatory CpG nucleic acids as adjuvants, as agents for use in treatment of asthma and allergy, and the like. Thus according to this embodiment of the invention, Th1-like skewing by immunostimulatory CpG nucleic acids can be inhibited. Such an effect may find use, for example, as an antidote to undesirable Th1-like skewing in the face of treatment with immunostimulatory CpG nucleic acids or exposure to immunostimulatory CpG nucleic acids through infection.

The step of contacting can take place before, essentially simultaneously with, or following contact of the cell with an appropriate source of immunostimulatory CpG nucleic acid molecule. For example, the contacting with the inhibitory ODN in certain embodiments takes place at least one day before the immune cell contacts a CpG nucleic acid molecule. As another example, the contacting with the inhibitory ODN in certain embodiments takes place at least one day after the immune cell contacts a CpG nucleic acid molecule. At least one day includes any time that is more than 24 hours and up to four weeks. In individual embodiments the at least one day is at least: 2 days, 3 days, 4 days, 5 days, 6 days, one week, two weeks, three weeks, or four weeks. In other embodiments the contacting with the inhibitory ODN can take place within 24 hours of the immune cell coming into contact with a CpG nucleic acid molecule.

It is believed that an effective amount of inhibitory ODN will generally be similar in amount to that of the source of CpG nucleic acid, although different amounts may be more or less effective. As disclosed in Example 8 below, inhibitory ODN of the invention were found to be highly effective inhibitors in vitro when present at concentrations between 1 and 100 percent of co-present TLR agonists. For use in vivo, an effective amount of inhibitory ODN may be higher.

In one embodiment the method is performed in vitro. In one embodiment the method is performed in vivo. Methods for assessing a reduction of an immunostimulatory effect of a CpG nucleic acid molecule are described above.

In one aspect the invention provides a method for treating a condition associated with CpG-mediated immunostimulation in a subject. The method according to this aspect of the invention involves the step of administering to a subject having or at risk of developing a condition associated with CpG-mediated immunostimulation an effective amount of an isolated immunoinhibitory nucleic acid molecule of the invention to treat the condition. The method is useful whenever it is desirable to skew an immune response away from a Th1-like immune response. According to this aspect of the invention, inhibitory ODN of the invention may be used to treat any of a number of conditions that involve an innate immune response or a Th1-like immune response, including inflammation, acute and chronic allograft rejection, graft-versus-host disease (GvHD), certain autoimmune diseases, infection, and sepsis.

Autoimmune diseases can be generally classified as antibody-mediated, T-cell mediated, or a combination of antibody-mediated and T-cell mediated. Inhibitory ODN of the invention are believed to be most useful for treating various types of autoimmunity involving antibody-mediated orT-cell mediated immunity, including insulin-dependent (type I) diabetes mellitus, rheumatoid arthritis, multiple sclerosis, systemic lupus erythematosus (SLE), and inflammatory bowel disease (i.e., Crohn's disease and ulcerative colitis). Animal models for these autoimmune diseases are available and are useful for assessing the efficacy of inhibitory ODN in these diseases. Other autoimmune diseases include, without limitation, alopecia areata, acquired hemophilia, ankylosing spondylitis, antiphospholipid syndrome, autoimmune hepatitis, autoimmune hemolytic anemia, Behçet's syndrome, cardiomyopathy, celiac sprue dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, discoid lupus, essential mixed cryoglobulinemia, fibromyalgia, fibromyositis, Guillain-Barré syndrome, idiopathic pulmonary fibrosis, idiopathic thrombocytopenic purpura, IgA nephropathy, juvenile arthritis, lichen planus, myasthenia gravis, polyarteritis nodosa, polychondritis, polyglandular syndromes, dermatomyositis, primary agammaglobulinemia, primary biliary cirrhosis, psoriasis, Raynaud's phenomena, Reiter's syndrome, sarcoidosis, stiff-man syndrome, Takayasu arthritis, temporal arteritis/giant cell arteritis, uveitis, vasculitis, and vitiligo.

In several autoimmune diseases antibodies to self antigens are frequently observed. For example for systemic lupus erythematosus autoantibodies have been described to single-stranded and double-stranded DNA or RNA. Vallin H et al. (1999) J Immunol 163:6306-13; Hoet R M et al. (1999) J Immunol 163:3304-12; ven Venrooij (1990) J Clin Invest 86:2154-60. The levels of autoantibodies found in the serum of autoimmune patients very often are found to correlate with disease severity. The pattern of autoantibodies that arise, e.g., in human SLE, suggest that intact macromolecular particles, such as RNA- or DNA-containing complexes, could themselves be immunogenic and anti-nucleic acid antibodies could therefore arise. Lotz M et al. (1992) Mol Biol Rep 16:127; Mohan C et al. (1993) J Exp Med 177:1367-81. Such DNA or RNA released from, e.g., apoptotic cells or DNA- or RNA-containing microbes present in serum of autoimmune patients, could be responsible for inflammation that contributes to the autoimmune disease. Fatenejad S (1994) J Immunol 152:5523-31; Malmegrim K C et al. (2002) Isr Med Assoc J 4:706-12; Newkirk M M et al. (2001) Arthritis Res 3:253-8. Indeed CpG-containing sequences could be identified from SLE serum that induces an efficient immune response dominated by IFN-α secretion that is thought to contribute the development of to autoimmune diseases. Magnusson M et al. (2001) Scand J Immunol 54:543-50; Rönnblom L et al. (2001) J Exp Med 194:F59-63. In addition, the epitopes for anti-RNA antibodies could be identified and are composed of G,U-rich sequences. Tsai D E et al. (1992) Proc Natl Acad Sci USA 89:8864-8; Tsai D E et al. (1993) J Immunol 150:1137-45. G,U-rich sequences appear to be natural ligands for TLR7 and TLR8 and, therefore, can mediate immune stimulatory responses that in principle could contribute to autoimmune diseases or the development of autoimmune diseases. PCT/US03/10406. Given the importance of immune stimulation mediated by serum CpG DNA or G,U-rich RNA that are targets for autoantibodies, the present invention provides a method for treating a condition associated with CpG DNA- or RNA-mediated immunostimulation in a subject having or being at risk of having an autoimmune disease.

Infections refer to any condition in which there is an abnormal collection or population of viable intracellular or extracellular microbes in a subject. Various types of microbes can cause infection, including microbes that are bacteria, microbes that are viruses, microbes that are fungi, and microbes that are parasites.

Bacteria include, but are not limited to, Pasteurella species, Staphylococci species, Streptococcus species, Escherichia coli, Pseudomonas species, and Salmonella species. Specific examples of infectious bacteria include but are not limited to, Helicobacter pyloris, Borrelia burgdorferi, Legionellapneumophilia, Mycobacteria sps (e.g., M tuberculosis, M. avium, M. intracellulare, M. kansasii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidum, Treponema pertenue, Leptospira, Rickettsia, and Actinomyces israelii.

Examples of viruses that have been found in humans include but are not limited to: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bunyaviridae (e.g., Hantaan viruses, bunga viruses, phleboviruses and Nairo viruses); Arenaviridae (hemorrhagic fever viruses); Reoviridae (e.g., reoviruses, orbiviurses and rotaviruses); Bornaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g., African swine fever virus); and unclassified viruses (e.g., the agent of delta hepatitis (thought to be a defective satellite of hepatitis B virus), Hepatitis C; Norwalk and related viruses, and astroviruses).

Fungi include yeasts and molds. Examples of fungi include without limitation Aspergillus spp including Aspergillus fumigatus, Blastomyces dermatitidis, Candida spp including Candida albicans, Coccidioides immitis, Cryptococcus neoformans, Histoplasma capsulatum, Pneumocystis carinii, Rhizomucor spp, and Rhizopus spp.

Other infectious organisms (i.e., protists) include Plasmodium spp. such as Plasmodium falciparum, Plasmodium malariae, Plasmodium ovale, and Plasmodium vivax and Toxoplasma gondii. Blood-borne and/or tissue parasites include Plasmodium spp., Babesia microti, Babesia divergens, Chlamydia trachomatis, Leishmania tropica, Leishmania spp., Leishmania braziliensis, Leishmania donovani, Trypanosoma gambiense and Trypanosoma rhodesiense (African sleeping sickness), Trypanosoma cruzi (Chagas' disease), and Toxoplasma gondii.

Other medically relevant microorganisms have been described extensively in the literature, e.g., see C. G. A Thomas, Medical Microbiology, Bailliere Tindall, Great Britain 1983, the entire contents of which is hereby incorporated by reference.

Dosing and Administration

The inhibitory ODN of the invention can be used alone, in combination with themselves, in combination with another agent, or in combination with themselves and with another agent. In addition to the conjugates described herein, the inhibitory ODN in combination with another agent can also be separate compositions that are used together to achieve a desired effect. For example, an inhibitory ODN and a second agent can be mixed together and administered to a subject or placed in contact with a cell as a combination. As another example, an inhibitory ODN and a second agent can be administered to a subject or placed in contact with a cell at different times. As yet another example, an inhibitory ODN and a second agent can be administered to a subject at different sites of administration.

The inhibitory ODN and/or the antigen and/or other therapeutics may be administered alone (e.g., in saline or buffer) or using any delivery vehicle known in the art. For instance the following delivery vehicles have been described: cochleates (Gould-Fogerite et al., 1994, 1996); emulsomes (Vancott et al., 1998, Lowell et al., 1997); ISCOMs (Mowat et al., 1993, Carlsson et al., 1991, Hu et., 1998, Morein et al., 1999); liposomes (Childers et al., 1999, Michalek et al., 1989, 1992, de Haan 1995a, 1995b); live bacterial vectors (e.g., Salmonella, Escherichia coli, bacillus Calmette-Guérin, Shigella, Lactobacillus) (Hone et al., 1996, Pouwels et al., 1998, Chatfield et al., 1993, Stover et al., 1991, Nugent et al., 1998); live viral vectors (e.g., Vaccinia, adenovirus, Herpes simplex) (Gallichan et al., 1993, 1995, Moss et al., 1996, Nugent et al., 1998, Flexner et al., 1988, Morrow et al., 1999); microspheres (Gupta et al., 1998, Jones et al., 1996, Maloy et al., 1994, Moore et al., 1995, O'Hagan et al., 1994, Eldridge et al., 1989); nucleic acid vaccines (Fynan et al., 1993, Kuklin et al., 1997, Sasaki et al., 1998, Okada et al., 1997, Ishii et al., 1997); polymers (e.g., carboxymethylcellulose, chitosan) (Hamajima et al., 1998, Jabbal-Gill et al., 1998); polymer rings (Wyatt et al., 1998); proteosomes (Vancott et al., 1998, Lowell et al., 1988, 1996, 1997); sodium fluoride (Hashi et al., 1998); transgenic plants (Tacket et al., 1998, Mason et al., 1998, Haq et al., 1995); Virosomes (Gluck et al., 1992, Mengiardi et al., 1995, Cryz et al., 1998); virus-like particles (Jiang et al., 1999, Leibl et al., 1998). Other delivery vehicles are known in the art.

As mentioned above, the term “effective amount” refers generally to the amount necessary or sufficient to realize a desired biologic effect. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular oligonucleotide being administered, the size of the subject, or the severity of the disease or condition. One of ordinary skill in the art can empirically determine the effective amount of a particular inhibitory ODN and/or antigen and/or other therapeutic agent without necessitating undue experimentation.

Subject doses of the compounds described herein for systemic or local delivery typically range from about 10 ng to 10 mg per administration, which depending on the application could be given daily, weekly, or monthly and any other amount of time therebetween or as otherwise required. More typically systemic or local doses range from about 1 μg to 1 mg per administration, and most typically from about 10 μg to 100 μg, with 2-4 administrations being spaced days or weeks apart. Higher doses may be required for parenteral administration. In some embodiments, however, parenteral doses for these purposes may be used in a range of 5 to 10,000 times higher than the typical doses described above.

For any compound described herein the therapeutically effective amount can be initially determined from animal models. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

Route of Administration

For clinical use the inhibitory ODN of the invention can be administered alone or formulated as a delivery complex via any suitable route of administration that is effective to achieve the desired therapeutic result. Routes of administration include enteral and parenteral routes of administration. Examples of enteral routes of administration include oral, gastric, intestinal, and rectal. Nonlimiting examples of parenteral routes of administration include intravenous, intramuscular, subcutaneous, intraperitoneal, intrathecal, local injection, topical, nasal, mucosal, and pulmonary.

Formulation

The inhibitory ODN of the invention may be directly administered to the subject or may be administered in conjunction with a nucleic acid delivery complex. A nucleic acid delivery complex shall mean a nucleic acid molecule associated with (e.g., ionically or covalently bound to; or encapsulated within) a targeting means (e.g., a molecule that results in higher affinity binding to target cell. Examples of nucleic acid delivery complexes include nucleic acids associated with a sterol (e.g., cholesterol), a lipid (e.g., a cationic lipid, virosome or liposome), or a target cell specific binding agent (e.g., a ligand recognized by target cell specific receptor). Preferred complexes may be sufficiently stable in vivo to prevent significant uncoupling prior to internalization by the target cell. However, the complex can be cleavable under appropriate conditions within the cell so that the oligonucleotide is released in a finctional form.

For oral administration, the compounds (i.e., inhibitory ODN, antigens and/or other therapeutic agents) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

The compounds may be administered by inhalation to pulmonary tract, especially the bronchi and more particularly into the alveoli of the deep lung, using standard inhalation devices. The compounds may be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. An inhalation apparatus may be used to deliver the compounds to a subject. An inhalation apparatus, as used herein, is any device for administering an aerosol, such as dry powdered form of the compounds. This type of equipment is well known in the art and has been described in detail, such as that description found in Remington: The Science and Practice of Pharmacy, 19^{th} Edition, 1995, Mac Publishing Company, Easton, Pa., pages 1676-1692. Many U.S. patents also describe inhalation devices, such as U.S. Pat. No. 6,116,237.

“Powder” as used herein refers to a composition that consists of finely dispersed solid particles. Preferably the compounds are relatively free flowing and capable of being dispersed in an inhalation device and subsequently inhaled by a subject so that the compounds reach the lungs to permit penetration into the alveoli. A “dry powder” refers to a powder composition that has a moisture content such that the particles are readily dispersible in an inhalation device to form an aerosol. The moisture content is generally below about 10% by weight (% w) water, and in some embodiments is below about 5% w and preferably less than about 3% w. The powder may be formulated with polymers or optionally may be formulated with other materials such as liposomes, albumin and/or other carriers.

Aerosol dosage and delivery systems may be selected for a particular therapeutic application by one of skill in the art, such as described, for example in Gonda, I. “Aerosols for delivery of therapeutic and diagnostic agents to the respiratory tract,” in Critical Reviews in Therapeutic Drug Carrier Systems, 6:273-313 (1990), and in Moren, “Aerosol dosage forms and formulations,” in Aerosols in Medicine. Principles, Diagnosis and Therapy, Moren, et al., Eds., Elsevier, Amsterdam, 1985.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may include suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R (1990) Science 249:1527-33, which is incorporated herein by reference.

The inhibitory ODN and optionally other therapeutics and/or antigens may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of an inhibitory ODN and optionally antigens and/or other therapeutic agents optionally included in a pharmaceutically acceptable carrier. The term pharmaceutically acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The present invention is further illustrated by the following Examples, which in no way should be construed as further limiting.

### EXAMPLES

### Example 1

### Importance of 5′ CC and GGG Motifs

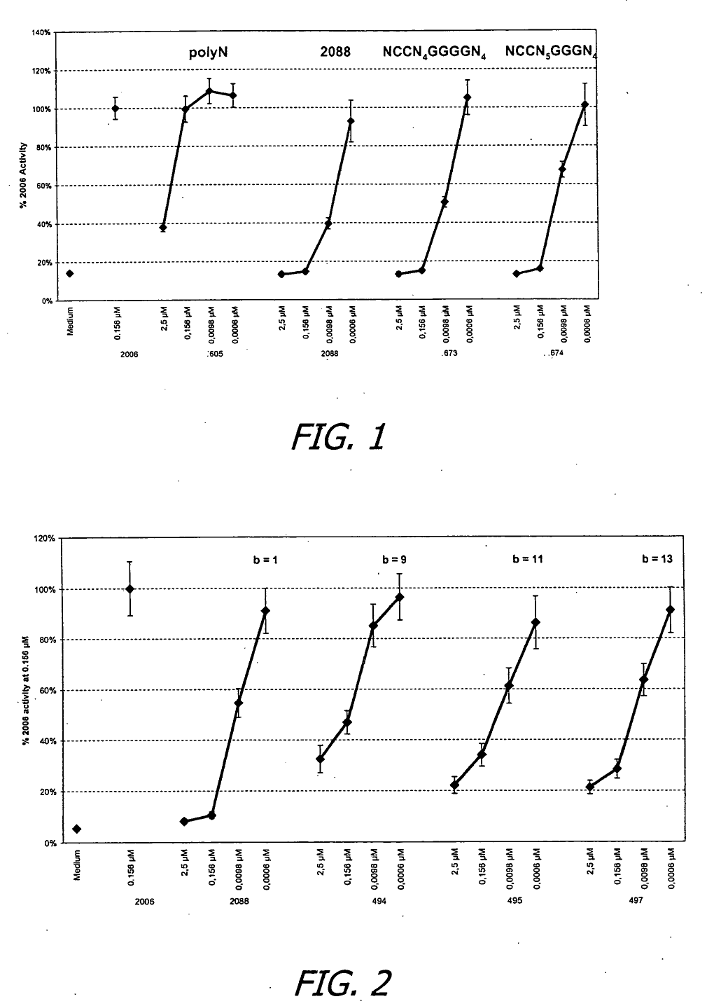

This experiment demonstrates the importance of the 5′ CC and GGG motifs to the inhibitory effect of inhibitory ODN that include a TLR9-antagonist motif. hTLR9-LUC-293 cells (human embryo kidney cells stably expressing human TLR9 and a 6× NF-κB-luciferase construct) were incubated with 0.156 μM CpG-ODN 2006 (TCGTCGTTTTGTCGTTTTGTCGTT; SEQ ID NO:17) and increasing amounts of inhibitory ODN 2088 (TCCTGGCGGGGAAGT; SEQ ID NO:4), 673 (NCCNNNNGGGGNNNN; SEQ ID NO:18), 674 (NCCNNNNNGGGNNNN; SEQ ID NO:19), or 605 (NNNNNNNNNNNNNN). Luciferase activity was measured 16 h later. Stimulation index was calculated in reference to medium activity. Activity of CpG ODN 2006 without addition of inhibitory ODN was set to 100%. Inhibition was expressed as remaining luciferase activity, without background correction for medium, i.e., background (medium) activity was 10%. Representative results are presented in FIG. 1.

As shown in FIG. 1, ODN 2088, 673, and 674, but not 605, effectively inhibited luciferase activity in hTLR9-LUC-293 cells in the presence of CpG ODN 2006. More specifically, percent activity of CpG ODN 2006 remaining at equimolar concentration of inhibitory ODN was 13 percent for each of 2088, 673, and 674, while the corresponding result for ODN 605 was 99 percent. These results indicate the importance of both the 5′ CC and the downstream GGG motif to the inhibitory effect of inhibitory ODN, and are consistent with previous reports. WO 00/14217.

### Example 2

### Length of Intervening Sequence between 5′ CC and GGG Motif

This experiment demonstrates the effect of increasing the value of b for an inhibitory ODN having a sequence XₐCCN₁N₂N₃Y_{b}N₄GGGZ_{c}. Experimental design was similar to that of Example 1, except the following inhibitory ODN were used: 2088 (b=1), 494 (b=9; TCCTGTGTGTGTGTCGGGGAAGT; SEQ ID NO:14), 495 (b=11; TCCTGTGTGTGTGTGTCGGGGAAGT; SEQ ID NO:15), and 497 (b=13; TCCTGTGTGTGTGTGTGTCGGGGAAGT; SEQ ID NO:16). In addition, the following ODN were also tested: 493, with a single Spacer 18 (TCCTGLGGGGAAGT; SEQ ID NO:37) and 492, with a 2× Spacer 18 (TCCTGLLGGGGAAGT; SEQ ID NO:38). Each Spacer 18 is a hexaethyleneglycol phosphate spacer commercially available from Glen Research. In yet additional experiments inhibitory ODN for which b=15, b=17, b=19, and b=21 are used. Representative results are presented in FIG. 2 and Table 1.

As shown in FIG. 2, increasing b from 1 to 9, 11, or 13 had little effect on inhibitory effect. More specifically, while ODN 2088 was generally more inhibitory than the other inhibitory ODN tested, ODN 494-497 were essentially similar in potency as inhibitors despite increasing b from 9 to 13. As shown in Table 1, when CpG ODN 2006 and inhibitory ODN were each present at 0.156 μM, percent activity of CpG ODN 2006 remaining was about 30-40 percent for each of ODN 494-497.

TABLE 1

Percent 2006 activity remaining at equimolar
concentrations (0.156 μM) of both immunostimulatory
CpG ODN 2006 and inhibitory ODN

| | | | | |
|---|---|---|---|---|
| | SEQ ID | | | Percent 2006 |
| ODN | NO: | Sequence | Remarks | Activity |

| | | | | |
|---|---|---|---|---|
| 2088 | 4 | TCCTGGCGGGGAAGT | b = 1 | 10 |
| 494 | 14 | TCCTGTGTGTGTGTCGGGGAAGT | b = 9 | 47 |
| 495 | 15 | TCCTGTGTGTGTGTGTCGGGGAAGT | b = 11 | 34 |
| 497 | 16 | TCCTGTGTGTGTGTGTGTGTCGGGGAAGT | b = 13 | 28 |
| 493 | 37 | TCCTGLGGGGAAGT | 1 = Spacer 18 | 54 |
| 492 | 38 | TCCTGLLGGGGAAGT | 2 = Spacer 18 | 67 |

### Example 3

### Replacement of the 5′ CC Motif by Any other Dinucleotide Results in Significant Loss of Inhibitory Effect

This set of experiments demonstrates the importance of the 5′ CC motif to the inhibitory effect of inhibitory ODN. Beginning with the sequence of ODN 2088, a complete series of related ODN having the 5′ CC dinucleotide replaced by other dinucleotides was made and tested for its ability to inhibit activity of CpG ODN 2006 as in Example 1. Representative results are presented in Table 2.

As shown in Table 2, substitution of the 5′ CC dinucleotide of ODN 2088 by any other dinucleotide resulted in a significant loss of inhibitory activity.

TABLE 2

Percent 2006 activity remaining at equimolar
concentrations (0.156 μM) of both immuno-
stimulatory CpG ODN 2006 and inhibitory ODN

| | | | |
|---|---|---|---|
| | SEQ ID | | |
| ODN | NO: | Sequence | Percent 2006 Activity |

| | | | |
|---|---|---|---|
| 959 | 39 | TAATGGCGGGGAAGT | 92 |
| 074 | 40 | TACTGGCGGGGAAGT | 132 |
| 441 | 41 | TAGTGGCGGGGAAGT | 105 |
| 442 | 42 | TATTGGCGGGGAAGT | 101 |
| 073 | 43 | TCATGGCGGGGAAGT | 114 |
| 2088 | 4 | TCCTGGCGGGGAAGT | 10 |
| 435 | 44 | TCGTGGCGGGGAAGT | 77 |
| 437 | 45 | TCTTGGCGGGGAAGT | 89 |
| 444 | 35 | TGATGGCGGGGAAGT | 96 |
| 443 | 34 | TGCTGGCGGGGAAGT | 91 |
| 436 | 46 | TGGTGGCGGGGAAGT | 90 |
| 445 | 36 | TGTTGGCGGGGAAGT | 97 |
| 439 | 47 | TTATGGCGGGGAAGT | 91 |
| 440 | 48 | TTCTGGCGGGGAAGT | 90 |
| 438 | 49 | TTGTGGCGGGGAAGT | 95 |
| 072 | 50 | TTTTGGCGGGGAAGT | 121 |

### Example 4

### Effect of Sequence Flanking the 5′ CC Dinucleotide

This set of experiments demonstrates relative insensitivity of inhibitory effect of inhibitory ODN to nucleotides immediately flanking the 5′ CC dinucleotide, as well as the significant loss of inhibitory effect of ODN to truncation of the 5′ CC dinucleotide. Beginning with the sequence of ODN 2088, a series of related ODN having the nucleotide 5′ or 3′ to the 5′ CC dinucleotide replaced by another single nucleotide was made and tested for its ability to inhibit activity of CpG ODN 2006 as in Example 1. In addition, in one test ODN (961), the nucleotide 5′ to the 5′ CC dinucleotide was deleted. In yet another test ODN 962 (SEQ ID NO:20), the nucleotide 5′ to the 5′ CC dinucleotide and the 5′ C of the 5′ CC dinucleotide were deleted. Representative results are presented in Table 3.

As shown in Table 3, inhibitory ODN were quite insensitive to substitution of one nucleotide flanking the 5′ CC dinucleotide for another. In addition, the inhibitory effect was essentially unchanged with deletion of the nucleotide 5′ to the 5′ CC dinucleotide. In contrast, however, deletion of both the nucleotide 5′ to the 5′ CC dinucleotide and the 5′ C of the 5′ CC dinucleotide resulted in significant loss of inhibitory activity.

TABLE 3

Percent 2006 activity remaining at equimolar
concentrations (0.156 μM) of both immuno-
stimulatory CpG ODN 2006 and inhibitory ODN

| | | | |
|---|---|---|---|
| | SEQ ID | | |
| ODN | NO: | Sequence | Percent 2006 Activity |

| | | | |
|---|---|---|---|
| 941 | 6 | ACCTGGCGGGGAAGT | 11 |
| 942 | 7 | CCCTGGCGGGGAAGT | 8 |
| 940 | 5 | GCCTGGCGGGGAAGT | 13 |
| 2088 | 4 | TCCTGGCGGGGAAGT | 10 |
| 156 | 9 | TCCGGGCGGGGAAGT | 10 |
| 155 | 8 | TCCCGGCGGGGAAGT | 11 |
| 157 | 51 | TCCAGGCGGGGAAGT | 14 |
| 961 | 52 | CCTGGCGGGGAAGT | 11 |
| 962 | 20 | CTGGCGGGGAAGT | 122 |

### Example 5

### Modifications of Cytidines in the 5′ CC Dinucleotide

This set of experiments demonstrates relative insensitivity of inhibitory effect of inhibitory ODN to substitution of modified forms of cytidine nucleotides in the 5′ CC dinucleotide. Beginning with the sequence of ODN 2088, a series of related ODN having the 5′ CC dinucleotide replaced with pairs of 2′-OMe-C, 5-Methyl-C, 5 Bromo-C, Ribo-C, 5-HO-C, or Ara-C was made and tested for its ability to inhibit activity of CpG ODN 2006 as in Example 1. Representative results are presented in Table 4.

As shown in Table 4, inhibitory ODN were relatively insensitive to the substitutions of cytidine nucleotides in the 5′ CC dinucleotide with cytidine derivatives.

TABLE 4

Percent 2006 activity remaining at equimolar
concentrations (0.156 μM) of both immuno-
stimulatory CpG ODN 2006 and inhibitory ODN

| | | | | |
|---|---|---|---|---|
| | | | | Percent |
| | SEQ ID | | | 2006 |
| ODN | NO: | Sequence | Remarks | Activity |

| | | | | |
|---|---|---|---|---|
| 2088 | 4 | TCCTGGCGGGGAAGT | | 10 |
| 166 | 33 | TCCTGGCGGGGAAGT | C = 2′-OMe-C | 11 |
| 159 | 32 | TZZTGGCGGGGAAGT | Z = 5-Methyl-C | 20 |
| 484 | 53 | TbCbCTGGCGGGGAA | bC = 5-Bromo-C | 22 |
| | | GT | | |
| 500 | 54 | TrCrCTGGCGGGGAA | rC = Ribo-C | 24 |
| | | GT | | |
| 483 | 55 | THHTGGCGGGGAAGT | H = 5-HO-C | 27 |
| 485 | 56 | TaCaCTGGCCGGGAA | aC = Ara-C | 56 |
| | | GT | | |

### Example 6

### Inhibitory ODN Selective for TLR8

The previous examples describe the ability of certain inhibitory ODN to inhibit activation of signal transduction mediated by human Toll-like receptor 9 (hTLR9). This set of experiments demonstrates the ability of certain inhibitory ODN to inhibit activation of signal transduction mediated by human Toll-like receptor 8 (hTLR8). The assay system involved stimulating hTLR8-LUC-293 cells with the TLR8 ligand, R-848 (Resiquimod; Jurk M et al. (2002) Nat Immunol 3:499) in the presence and absence of ODN and comparing the induction of luciferase activity in the absence of ODN to luciferase activity in the presence of ODN.

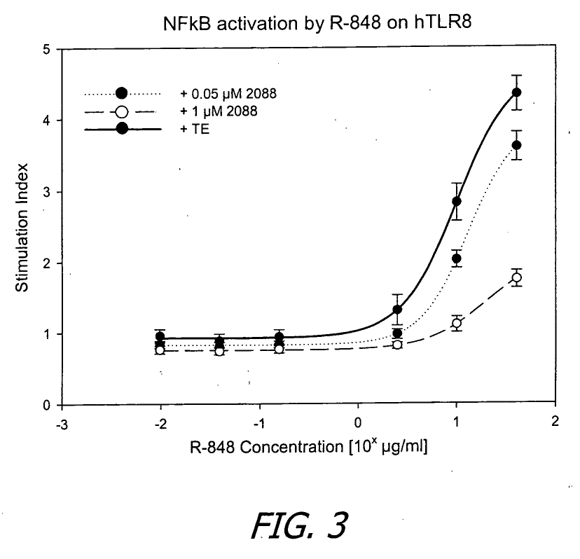

In a first experiment hTLR8-LUC-293 cells (human embryo kidney cells stably expressing human TLR8 and a 6× NF-κB-luciferase construct) were incubated with increasing amounts of R-848 in the absence or presence of constant amounts of ODN 2088 (TCCTGGCGGGGAAGT; SEQ ID NO:4) or ODN 2114 (TCCTGGAGGGGAAGT; SEQ ID NO:11) ranging from 0 to 1 μM. Cells were assayed for luciferase activity 16 h later. Results were calculated as fold induction above medium background. Representative results are shown in FIG. 3.

As shown in FIG. 3, ODN 2088 inhibited R-848-mediated NF-κB activation in hTLR8-LUC-293 cells. Similar results were obtained using ODN 2114 instead of ODN 2088.

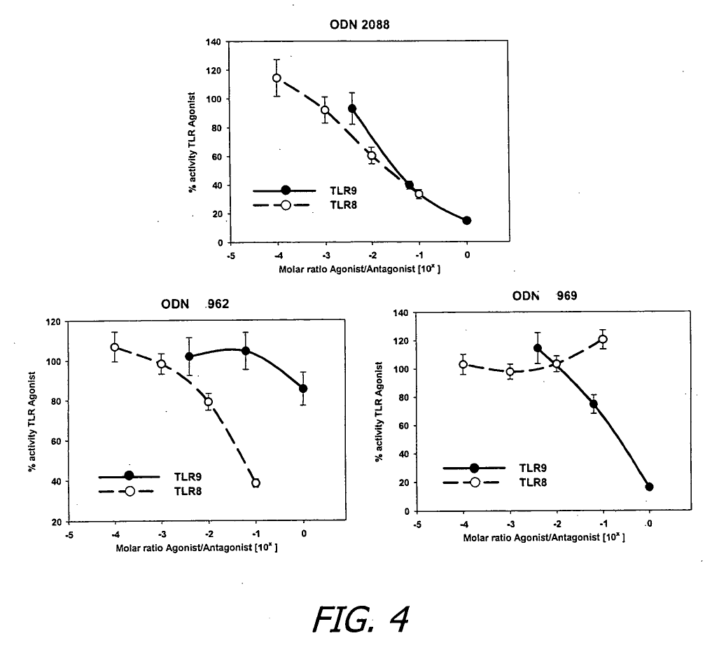

Surprisingly, follow-up experiments showed that certain ODN specifically inhibited hTLR8 activity but did not inhibit hTLR9 activity. Additional follow-up experiments showed that certain ODN specifically inhibited hTLR9 activity but not hTLR8 activity. hTLR8-LUC-293 and hTLR9-LUC-293 cells were incubated with a constant amount of R-848 (50 μM) and CpG ODN 2006 (0.156 μM), respectively, in the presence of increasing concentrations of ODN 2088, ODN 962 (CTGGCGGGGAAGT; SEQ ID NO:20), or ODN 969 (TCCTGGCGGGGAA; SEQ ID NO:21). Luciferase activity was assayed after 16 h. Representative results are shown in FIG. 4.

As shown in FIG. 4A, ODN 2088 inhibited both TLR-mediated NF-κB signaling pathways, on hTLR9 as well as on hTLR8. In contrast, as shown in FIG. 4B, ODN 962 showed specific inhibition on hTLR8 while incubation of ODN 692 with CpG ODN 2006 did not result in any inhibition on hTLR9. As shown in FIG. 4C, ODN 969 inhibited hTLR9 but not TLR8, confirming the existence of different inhibitory sequences effective for inhibiting TLR8 and TLR9.

### Example 7

### TLR8-Antagonist Motif

Based on preliminary studies including those in Example 6, it was determined that the effect of inhibition of TLR8-mediated NF-κB activation was associated with sequences having the formula Nₓ*G*K, wherein K=T≈U>G; x=0-27; G is guanosine or 7-deazaguanosine; and N can be any nucleotide or dSpacer. Long stretches of polynucleotides (e.g., >4 successive T or A were found to reduce inhibitory capacity of the ODN significantly). Generally, activity appeared to vary with backbone as follows: phosphorothioate>2′-OMe>all phosphodiester.

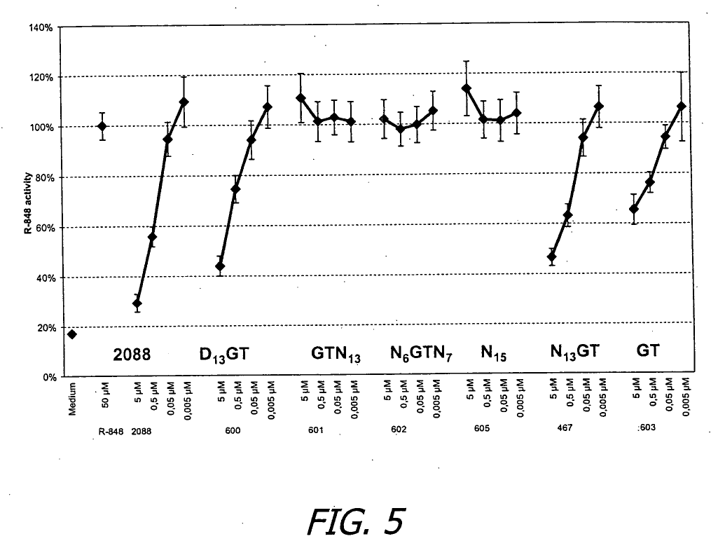

hTLR8-LUC-293 cells were incubated with 50 μM R-848 in the presence of increasing concentrations of the following ODN: ODN 2088, D₁₃GT (whererin D is dSpacer), GTN₁₃, N₆GTN₇, random 15-mer N₁₅, N₁₃GT, and the dinucleotide GT. Luciferase activity was assayed after 16 h. Luciferase activity of R-848 without any ODN was set to 100% (without correction for medium background, i.e. background activity of medium remained at approx. 17%). Representative results are shown in FIG. 5.

As shown in FIG. 5, ODN containing the dinucleotide motif GT at the very 3′ end displayed inhibition of hTLR8-mediated NF-κB activation. The dinucleotide GT (x=0) was also active in inhibition. ODN containing a GT motif at the 5′-end or in the interior of the sequence did not suppress R-848 activity in this assay.

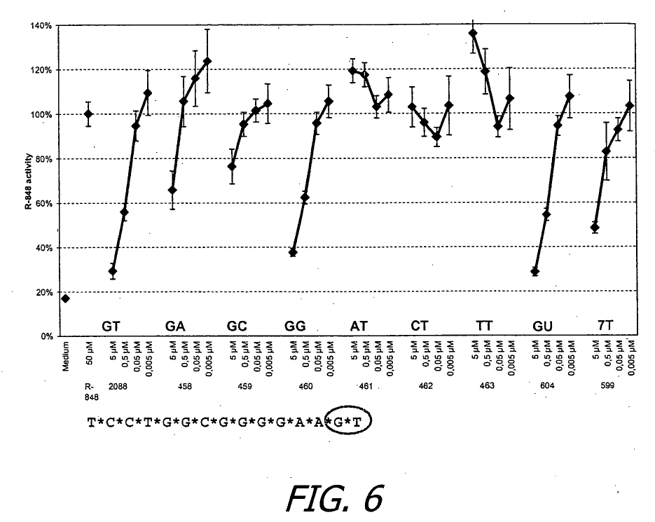

In a first of experiments hTLR8-LUC-293 cells were incubated with 50 μM R-848 in the presence of increasing concentrations of ODN 2088 or the following ODN, in which the terminal 3′ GT dinucleotide of ODN 2088 was replaced by another dinucleotide: ODN 458 (TCCTGGCGGGAAGA; SEQ ID NO:22), ODN 459 (TCCTGGCGGGGAAGC; SEQ ID NO:23), ODN 460 (TCCTGGCGGGGAAGG; SEQ ID NO:24), ODN 461 (TCCTGGCGGGGAAAT; SEQ ID NO:25), ODN 462 (TCCTGGCGGGGAACT; SEQ ID NO:26), 463 (TCCTGGCGGGGAATT; SEQ ID NO;27), 604 (TCCTGGCGGGGAAGU; SEQ ID NO:28), and ODN 599 (TCCTGGCGGGGAA7T; SEQ ID NO:29). Luciferase activity was assayed after 16 h. Luciferase activity of R-848 without any ODN was set to 100% (without correction for medium background, i.e., background activity of medium remained at approx. 17%). Representative results are shown in FIG. 6.

As shown in FIG. 6, the G of the terminal 3′ GT dinucleotide can be replaced by 7-deaza G, whereas the T can be replaced by U and (less efficiently) by G.

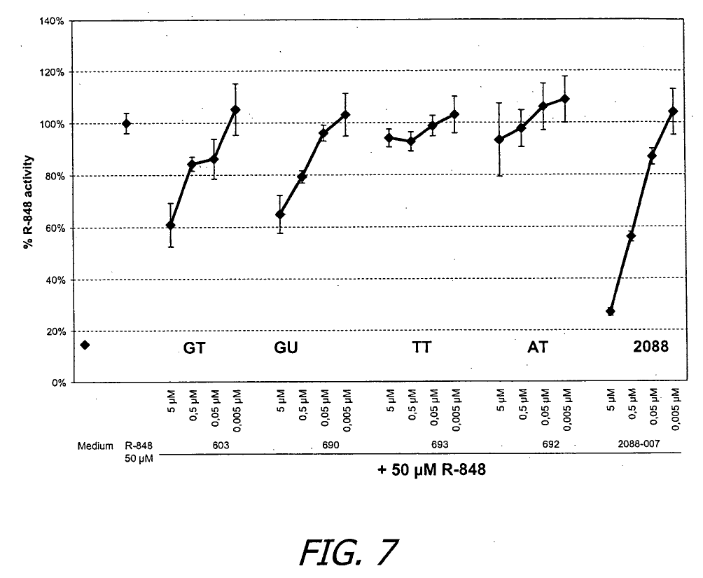

In yet a further set of experiments hTLR8-LUC-293 cells were incubated with 50 μM R-848 in the presence of increasing amounts of different dinucleotides (GT, ODN 603; TG, ODN 688; GG, ODN 689; GU, ODN 690; UG, ODN 691; AT, ODN 692; and TT, ODN 693). Luciferase was assayed after 16 h. Luciferase activity of R-848 without any ODN was set to 100% and remaining R-848-mediated NF-κB activation in the presence of dinucleotides or ODN 2088 was calculated. Medium background was 17% in this set of experiments. Representative results are presented in Table 5 and FIG. 7.

TABLE 5

Percent R-848 activity remaining at 10-fold
molar excess of agonist (R-848, 50 μM)
to antagonist (ODN, 5 μM)

| | | |
|---|---|---|
| ODN | Sequence | Percent Activity |

| | | |
|---|---|---|
| 2088 | TCCTGGCGGGGAAGT | 27.0 |
| 603 | GT | 60.9 |
| 690 | GU | 64.9 |
| 692 | AT | 93.0 |
| 693 | TT | 94.1 |
| 691 | UG | 98.8 |
| 689 | GG | 117.0 |
| 688 | TG | 122.0 |

As shown in FIG. 7, of the dinucleotides tested, only the dinucleotides GT and GU showed significant inhibition at 5 μM concentration, confirming the importance of 3′ GT or GU.

### Example 8

### Different Sequence Requirements for Inhibitors of TLR7, TLR8, and TLR9

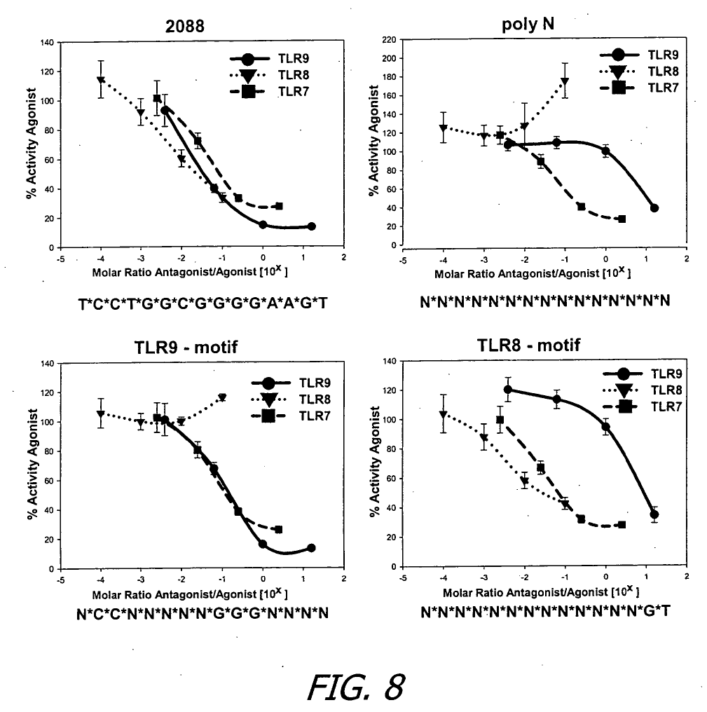

hTLR-LUC-293 cells (expressing the respective humanTLR7, TLR8, or TLR9) were incubated with constant amounts of TLR-agonist (for hTLR7: 2 μM R-848, for hTLR8: 50 μM R-848, and for hTLR9: 0.156 μM CpG ODN 2006) and increasing amounts of TLR-antagonist. TLR antagonists were ODN 2088, random 15-mer (poly N), NCCNNNNNGGGNNNN (SEQ ID NO:19), and NNNNNNNNNNNNNGT. To compare data for different TLRs, percent remaining activity of agonist was plotted against the log of molar ratio of antagonist to agonist (e.g., for hTLR8: 50 μM R-848 and 5 μM ODN, the plotted ratio is 10⁻¹). Used ODN concentrations were: for hTLR7 and hTLR8, 5 μM, 0.5 μM, 0.05 μM, and 0.005 μM, and for hTLR9, 2.5 μM, 0.156 μM, 0.0098 μM and 0.00061 μM. Representative results are presented in FIG. 8.

As shown in FIG. 8, huTLR7 was inhibited unspecifically by any phosphorothioate ODN. However, huTLR8 was not inhibited by ODN having a motif specific for inhibitors of TLR9 but not TLR8. Furthermore, huTLR9 was not inhibited by ODN having a motif specific for inhibitors of TLR7 but not TLR8 or TLR9, demonstrating the specificity of inhibition of huTLR8 and huTLR9.

### Example 9

### Effects of Inhibitory ODN in Cells Naturally Expressing TLR

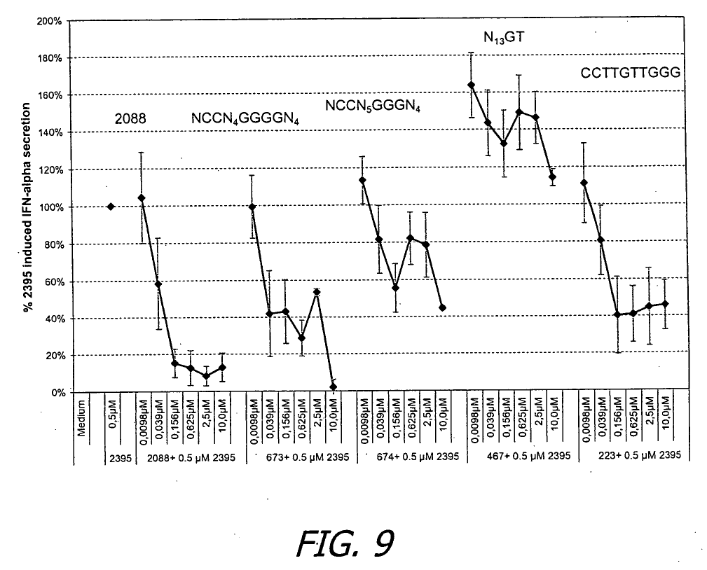

Examples 1-8 above demonstrate effects of the inhibitory ODN of the invention on TLRs expressed artificially in TLR-transfected cells. In this set of experiments human PBMC were isolated from human blood and incubated in the presence of 0.5 μM CpG ODN 2395 (TCGTCTTTTCGGCGCGCGCCG; SEQ ID NO:30) and increasing concentrations of inhibitory ODN. CpG ODN 2395 induces high amounts of IFN-α in human PBMC through TLR9-mediated signaling. The secreted amount of IFN-α induced by CpG ODN 2395 after 24 h was set to 100%. Amounts of IFN-α produced in the presence of 0.5 μM CpG ODN 2395 and increasing concentrations of ODN 2088 (SEQ ID NO:4), ODN 673 (NCCNNNNGGGGNNNN; SEQ ID NO:18), ODN 674 (NCCCNNNNNGGGNNNN; SEQ ID NO:19), ODN 467 (NNNNNNNNNNNNNGT) and ODN 223 (CCTTGTTGGG; SEQ ID NO:31) were measured and calculated in reference to IFN-α amounts induced by CpG ODN 2395 without addition of any other ODN. Representative results are presented in FIG. 9.

As shown in FIG. 9, IFN-α production in human PBMC was inhibited by ODNs 2088 and 673, 674, and 223, each containing a motif specific for TLR9, but not by an ODN containing a sequence motif specific for TLR8 (ODN 467).

### Equivalents

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages of the invention are not necessarily encompassed by each embodiment of the invention.

All references, patents and patent publications that are recited in this application are incorporated in their entirety herein by reference.

1. A composition comprising an isolated immunoinhibitory nucleic acid molecule comprising a sequence
XₐCCN₁N₂N₃Y_{b}N₄GGGZ_{c} (SEQ ID NO:1)
wherein:
each C is cytidine or a derivative thereof, wherein at least one C is a cytidine derivative;
each G is guanosine or a deaza derivative thereof;
Xₐ is any nucleotide sequence a nucleotides long, wherein a is an integer between 0-12, inclusive, and each nucleotide is selected independently of any other in Xₐ;
Y_{b} is any nucleotide sequence b nucleotides long, wherein b is an integer between 0-21, inclusive, and each nucleotide is selected independently of any other in Y_{b};
Z_{c}is any nucleotide sequence c nucleotides long, wherein c is an integer between 0-12, inclusive, and each nucleotide is selected independently of any other in Z_{c}; and
N₁, N₂, N₃, and N₄ are each independently any nucleotide:

2. The composition of claim 1, wherein N₁ is T (thymidine).

3. The composition of claim 1, wherein N₂ is G.

4. The composition of claim 1, wherein N₁N₂ is TG.

5. The composition of claim 1, wherein N₄ is G.

6. The composition of claim 1, wherein Xₐ is T.

7. The composition of claim 1, wherein each C is a cytidine derivative.

8. The composition of claim 1, wherein at least one C is 5-methylcytidine.

9. The composition of claim 1, wherein at least one G is 7-deazaguanosine.

10. The composition of claim 1, wherein each G is 7-deazaguanosine.

11. The composition of claim 1, wherein b is a smallest integer between 0-21, inclusive, able to conform to the sequence.

12. The composition of claim 1, wherein the immunoinhibitory nucleic acid molecule has a phosphorothioate backbone.

13. The composition of claim 1, wherein the sequence comprises XₐCCTGN₃Y_{b}GGGGZ_{c} (SEQ ID NO:3).

14-23. (canceled)

24. The composition of claim 1, further comprising a pharmaceutically acceptable carrier.

25. A composition comprising an isolated immunoinhibitory nucleic acid molecule comprising a sequence
XₐCCN₁N₂N₃Y_{b}N₄GGGZ_{c} (SEQ ID NO:1)
wherein:
each C is cytidine or a derivative thereof;
each G is guanosine or a deaza derivative thereof;
Xₐ is any nucleotide sequence a nucleotides long, wherein a is an integer between 0-12, inclusive, and each nucleotide is selected independently of any other in Xₐ;
Y_{b} is any nucleotide sequence b nucleotides long, wherein b is an integer between 8-21, inclusive, and each nucleotide is selected independently of any other in Y_{b};
Z_{c}is any nucleotide sequence c nucleotides long, wherein c is an integer between 0-12, inclusive, and each nucleotide is selected independently of any other in Z_{c}; and
N₁, N₂, N₃, and N₄ are each independently any nucleotide.

26-39. (canceled)

40. A composition comprising an isolated immunoinhibitory nucleic acid molecule comprising a sequence
XₐCCN₁Y_{b}N₂N₃N₄N₅GGZ_{c} (SEQ ID NO:2)
wherein:
N₂N₃N₄N₅GG is selected from GGN₄N₅GG, GN₃N₄GGG, N₂GN₄GGG, and N₂N₃N₄GGG;
each C is cytidine or a derivative thereof;
each G is guanosine or a deaza derivative thereof;
Xₐ is any nucleotide sequence a nucleotides long, wherein a is an integer between 0-12, inclusive, and each nucleotide is selected independently of any other in Xₐ;
Y_{b} is any nucleotide sequence b nucleotides long, wherein b is an integer between 8-21, inclusive, and each nucleotide is selected independently of any other in Y_{b};
Z_{c}is any nucleotide sequence c nucleotides long, wherein c is an integer between 0-12, inclusive, and each nucleotide is selected independently of any other in Z_{c}; and
N₁, N₂, N₃, N₄, and N₅ are each independently any nucleotide.

41-48. (canceled)

49. A composition comprising a conjugate of an antigen and an isolated immunoinhibitory nucleic acid molecule of claim 1.

50. A composition comprising a conjugate of a TLR agonist and an isolated immunoinhibitory nucleic acid molecule of claim 1.

51. The composition of claim 1, wherein Z_{c}is not K when c is 1 and wherein Z_{c}does not terminate with GK when c is an integer between 2-12, inclusive, wherein G is chosen from guanosine and 7-deazaguanosine and K is chosen from thymidine (T), uracil (U), and G.

52. The composition of claim 1, wherein Z_{c}is not T when c is 1 and wherein Z_{c}does not terminate with GT when c is an integer between 2-12, inclusive, wherein G is guanosine and T is thymidine.

53. The composition of claim 12, wherein Z_{c}is K when c is 1 and wherein Z_{c} terminates with GK when c is an integer between 2-12, inclusive, wherein G is chosen from guanosine and 7-deazaguanosine and K is chosen from thymidine (T), uracil (U), and G.

54. The composition of claim 12, wherein Z_{c}is T when c is 1 and wherein Z_{c}terminates with GT when c is an integer between 2-12, inclusive, wherein G is guanosine and T is thymidine.

55. The composition of claim 12, wherein Z_{c}terminates with 7T when c is an integer between 2-12, inclusive, wherein 7 is 7-deazaguanosine.

56. A method for inhibiting TLR signaling, comprising:
contacting a cell or a population of cells expressing at least one TLR chosen from TLR7, TLR8, TLR9, or any combination thereof, with an effective amount of a composition of claim 53 to inhibit signaling by TLR7, TLR8, and TLR9.

57. (canceled)

58. A method for inhibiting TLR9 signaling, comprising:
contacting a cell or a population of cells expressing TLR9 with an effective amount of a composition of claim 1, to inhibit signaling by TLR9.

59. (canceled)

60. A method for inhibiting TLR8 signaling, comprising:
contacting a cell or a population of cells expressing TLR8 with an effective amount of a GK dinucleotide, wherein G is chosen from guanosine and 7-deazaguanosine and K is chosen from thymidine, uracil, and guanosine, to inhibit signaling by TLR8.

61. (canceled)

62. A method for inhibiting TLR8 signaling, comprising:
contacting a cell or a population of cells expressing TLR8 with an effective amount of a GT dinucleotide, wherein G is guanosine T is thymidine, to inhibit signaling by TLR8.

63. (canceled)

64. A method for inhibiting TLR signaling, comprising:
contacting a cell or a population of cells expressing at least one TLR chosen from TLR7, TLR8, TLR9, or any combination thereof, with an effective amount of a composition of claim 51 to inhibit signaling by TLR9; and
contacting the cell or population of cells with an effective amount of a phosphorothioate oligonucleotide 2-40 nucleotides long comprising a 3′ end terminating with GK, wherein G is chosen from guanosine and 7-deazaguanosine and K is chosen from thymidine, uracil, and guanosine, to inhibit signaling by TLR7 and TLR8.

65. (canceled)

66. A method for inhibiting TLR9 signaling without inhibiting TLR8 signaling, comprising:
contacting a cell or a population of cells expressing TLR8 and TLR9 with an effective amount of a composition of claim 51 to inhibit signaling by TLR9 without inhibiting signaling by TLR8.

67. (canceled)

68. A method for promoting TLR9 signaling and inhibiting TLR8 signaling, comprising:
contacting a cell or population of cells expressing TLR8 and TLR9 with an effective amount of an immunostimulatory CpG nucleic acid molecule, wherein the immunostimulatory CpG nucleic acid molecule does not have a 3′ end terminating with GT, to promote TLR9 signaling; and
contacting the cell or population of cells with an effective amount of an oligonucleotide 2-40 nucleotides long, wherein the oligonucleotide comprises a 3′ end terminating with GT, to inhibit signaling by TLR8.

69. (canceled)

70. A method for promoting TLR8 signaling and inhibiting TLR9 signaling, comprising:
contacting a cell or population of cells expressing TLR8 and TLR9 with an effective amount of a TLR8 signaling agonist to promote TLR8 signaling; and
contacting the cell or population of cells with an effective amount of a composition of claim 1, to inhibit TLR9 signaling.

71. (canceled)

72. A method for reducing an immunostimulatory effect of a CpG nucleic acid molecule, comprising:
contacting an immune cell that is sensitive to a CpG nucleic acid molecule with an effective amount of an isolated immunoinhibitory nucleic acid molecule of claim 1 to reduce an immunostimulatory effect of the CpG nucleic acid molecule on the immune cell to a level below that which would occur without the contacting.

73-76. (canceled)

77. A method for treating a condition associated with CpG-mediated immunostimulation in a subject, comprising:
administering to a subject having or at risk of developing a condition associated with CpG-mediated immunostimulation an effective amount of an isolated immunoinhibitory nucleic acid molecule of claim 1 to treat the condition.

78-85. (canceled)


SEQUENCE LISTING (58 sequences)

SEQ ID NO: 1 - DNA, 54 bp, Artificial sequence
  Synthetic oligonucleotide
nnnnnnnnnnnnccnnnnnnnnnnnnnnnnnnnnnnnnngggnnnnnnnnnnnn

SEQ ID NO: 2 - DNA, 54 bp, Artificial sequence
  Synthetic oligonucleotide
nnnnnnnnnnnnccnnnnnnnnnnnnnnnnnnnnnnnnnnggnnnnnnnnnnnn

SEQ ID NO: 3 - DNA, 54 bp, Artificial sequence
  Synthetic oligonucleotide
nnnnnnnnnnnncctgnnnnnnnnnnnnnnnnnnnnnnggggnnnnnnnnnnnn

SEQ ID NO: 4 - DNA, 15 bp, Artificial sequence
  Synthetic oligonucleotide
tcctggcggggaagt

SEQ ID NO: 5 - DNA, 15 bp, Artificial sequence
  Synthetic oligonucleotide
gcctggcggggaagt

SEQ ID NO: 6 - DNA, 15 bp, Artificial sequence
  Synthetic oligonucleotide
acctggcggggaagt

SEQ ID NO: 7 - DNA, 15 bp, Artificial sequence
  Synthetic oligonucleotide
ccctggcggggaagt

SEQ ID NO: 8 - DNA, 15 bp, Artificial sequence
  Synthetic oligonucleotide
tcccggcggggaagt

SEQ ID NO: 9 - DNA, 15 bp, Artificial sequence
  Synthetic oligonucleotide
tccgggcggggaagt

SEQ ID NO: 10 - DNA, 15 bp, Artificial sequence
  Synthetic oligonucleotide
tcctagcggggaagt

SEQ ID NO: 11 - DNA, 15 bp, Artificial sequence
  Synthetic oligonucleotide
tcctggaggggaagt

SEQ ID NO: 12 - DNA, 20 bp, Artificial sequence
  Synthetic oligonucleotide
tcctagcgggggcgtcctat

SEQ ID NO: 13 - DNA, 16 bp, Artificial sequence
  Synthetic oligonucleotide
cctcaagcttgagggg

SEQ ID NO: 14 - DNA, 23 bp, Artificial sequence
  Synthetic oligonucleotide
tcctgtgtgtgtgtcggggaagt

SEQ ID NO: 15 - DNA, 25 bp, Artificial sequence
  Synthetic oligonucleotide
tcctgtgtgtgtgtgtcggggaagt

SEQ ID NO: 16 - DNA, 27 bp, Artificial sequence
  Synthetic oligonucleotide
tcctgtgtgtgtgtgtgtcggggaagt

SEQ ID NO: 17 - DNA, 24 bp, Artificial sequence
  Synthetic oligonucleotide
tcgtcgttttgtcgttttgtcgtt

SEQ ID NO: 18 - DNA, 15 bp, Artificial sequence
  Synthetic oligonucleotide
nccnnnnggggnnnn

SEQ ID NO: 19 - DNA, 15 bp, Artificial sequence
  Synthetic oligonucleotide
nccnnnnngggnnnn

SEQ ID NO: 20 - DNA, 13 bp, Artificial sequence
  Synthetic oligonucleotide
ctggcggggaagt

SEQ ID NO: 21 - DNA, 13 bp, Artificial sequence
  Synthetic oligonucleotide
tcctggcggggaa

SEQ ID NO: 22 - DNA, 15 bp, Artificial sequence
  Synthetic oligonucleotide
tcctggcggggaaga

SEQ ID NO: 23 - DNA, 15 bp, Artificial sequence
  Synthetic oligonucleotide
tcctggcggggaagc

SEQ ID NO: 24 - DNA, 15 bp, Artificial sequence
  Synthetic oligonucleotide
tcctggcggggaagg

SEQ ID NO: 25 - DNA, 15 bp, Artificial sequence
  Synthetic oligonucleotide
tcctggcggggaaat

SEQ ID NO: 26 - DNA, 15 bp, Artificial sequence
  Synthetic oligonucleotide
tcctggcggggaact

SEQ ID NO: 27 - DNA, 15 bp, Artificial sequence
  Synthetic oligonucleotide
tcctggcggggaatt

SEQ ID NO: 28 - DNA, 15 bp, Artificial sequence
  Synthetic oligonucleotide
tcctggcggggaagn

SEQ ID NO: 29 - DNA, 15 bp, Artificial sequence
  Synthetic oligonucleotide
tcctggcggggaant

SEQ ID NO: 30 - DNA, 21 bp, Artificial sequence
  Synthetic oligonucleotide
tcgtcttttcggcgcgcgccg

SEQ ID NO: 31 - DNA, 10 bp, Artificial sequence
  Synthetic oligonucleotide
ccttgttggg

SEQ ID NO: 32 - DNA, 15 bp, Artificial sequence
  Synthetic oligonucleotide
tnntggcggggaagt

SEQ ID NO: 33 - DNA, 15 bp, Artificial sequence
  Synthetic oligonucleotide
tnntggcggggaagt

SEQ ID NO: 34 - DNA, 15 bp, Artificial sequence
  Synthetic oligonucleotide
tgctggcggggaagt

SEQ ID NO: 35 - DNA, 15 bp, Artificial sequence
  Synthetic oligonucleotide
tgatggcggggaagt

SEQ ID NO: 36 - DNA, 15 bp, Artificial sequence
  Synthetic oligonucleotide
tgttggcggggaagt

SEQ ID NO: 37 - DNA, 14 bp, Artificial sequence
  Synthetic oligonucleotide
tcctgnggggaagt

SEQ ID NO: 38 - DNA, 15 bp, Artificial sequence
  Synthetic oligonucleotide
tcctgnnggggaagt

SEQ ID NO: 39 - DNA, 15 bp, Artificial sequence
  Synthetic oligonucleotide
taatggcggggaagt

SEQ ID NO: 40 - DNA, 15 bp, Artificial sequence
  Synthetic oligonucleotide
tactggcggggaagt

SEQ ID NO: 41 - DNA, 15 bp, Artificial sequence
  Synthetic oligonucleotide
tagtggcggggaagt

SEQ ID NO: 42 - DNA, 15 bp, Artificial sequence
  Synthetic oligonucleotide
tattggcggggaagt

SEQ ID NO: 43 - DNA, 15 bp, Artificial sequence
  Synthetic oligonucleotide
tcatggcggggaagt

SEQ ID NO: 44 - DNA, 15 bp, Artificial sequence
  Synthetic oligonucleotide
tcgtggcggggaagt

SEQ ID NO: 45 - DNA, 15 bp, Artificial sequence
  Synthetic oligonucleotide
tcttggcggggaagt

SEQ ID NO: 46 - DNA, 15 bp, Artificial sequence
  Synthetic oligonucleotide
tggtggcggggaagt

SEQ ID NO: 47 - DNA, 15 bp, Artificial sequence
  Synthetic oligonucleotide
ttatggcggggaagt

SEQ ID NO: 48 - DNA, 15 bp, Artificial sequence
  Synthetic oligonucleotide
ttctggcggggaagt

SEQ ID NO: 49 - DNA, 15 bp, Artificial sequence
  Synthetic oligonucleotide
ttgtggcggggaagt

SEQ ID NO: 50 - DNA, 15 bp, Artificial sequence
  Synthetic oligonucleotide
ttttggcggggaagt

SEQ ID NO: 51 - DNA, 15 bp, Artificial sequence
  Synthetic oligonucleotide
tccaggcggggaagt

SEQ ID NO: 52 - DNA, 14 bp, Artificial sequence
  Synthetic oligonucleotide
cctggcggggaagt

SEQ ID NO: 53 - DNA, 15 bp, Artificial sequence
  Synthetic oligonucleotide
tnntggcggggaagt

SEQ ID NO: 54 - DNA, 15 bp, Artificial sequence
  Synthetic oligonucleotide
tnntggcggggaagt

SEQ ID NO: 55 - DNA, 15 bp, Artificial sequence
  Synthetic oligonucleotide
tnntggcggggaagt

SEQ ID NO: 56 - DNA, 15 bp, Artificial sequence
  Synthetic oligonucleotide
tnntggcggggaagt

SEQ ID NO: 57 - DNA, 21 bp, Artificial sequence
  Synthetic oligonucleotide
ctcctagcgggggcgtcctat

SEQ ID NO: 58 - DNA, 54 bp, Artificial sequence
  Synthetic oligonucleotide
nnnnnnnnnnnnccthgnnnnnnnnnnnnnnnnnnnnnggggnnnnnnnnnnnn